(19) United States
(12) Patent Application Publication
(10) Pub. No.: US 2025/0313820 A1
(43) Pub. Date: Oct. 9, 2025

(54) PEPTIDES WITH MULTIVALENT EFFECTS

(71) Applicant: In2Cure AB, Lund (SE)

(72) Inventors: Artur Schmidtchen, Lund (SE); Ganna Petruk, Lund (SE)

(21) Appl. No.: 18/702,831

(22) PCT Filed: Oct. 21, 2022
(86) PCT No.: PCT/EP2022/079429

§ 371 Date: Apr. 19, 2024

(30) Foreign Application Priority Data
Oct. 22, 2021 (EP) 21204274.1

(51) Int. Cl. C12N 9/74; A61K 38/00; A61P 7/02; A61P 11/00; A61P 29/00

(52) U.S. Cl. CPC C12N 9/6429; A61P 7/02; A61P 11/00; A61P 29/00; A61K 38/00; C12Y 304/21005

(57) ABSTRACT

The invention relates to thrombin derived peptides comprising at least one internal covalent linkage between the side chains of two non-neighbouring, internal amino acids. The peptides have anti-inflammatory effect.

### CROSS REFERENCE TO RELATED APPLICATIONS

This application is a U.S. National Stage under 35 U.S.C. § 371 of PCT/EP2022/079429 filed Oct. 21, 2022, which depends from and claims priority to European patent application number 21204274.1 filed Oct. 22, 2021, the entire contents of which are incorporated herein by reference.

### SEQUENCE LISTING

The instant application contains a Sequence Listing which has been submitted in .xml format via Patent Center and is hereby incorporated by reference in its entirety. Said .xml copy, created on Oct. 23, 2024, is named 2024-11-01 Substitute Sequence Listing INCU0004PA.xml and is 81,815 bytes in size.

### TECHNICAL FIELD

The present invention lies within the field of peptides for treatment of inflammation and/or infection. In particular, the invention provides peptides with good stability, high anti-inflammatory activity and/or anti-microbial activity.

### BACKGROUND

Lipopolysaccharide (LPS) sensing by Toll-like receptor 4 (TLR4) is crucial in early responses to infection, and the subsequent NFkB activation causes a variety of biological effects associated with sepsis and ARDS, including release of cytokines, chemokines, and subsequent detrimental hemostatic disturbances, leading to consumption of coagulation factors and other mediators. Interestingly, spike glycoprotein, the major extracellular protein of SARS-CoV-2, boosts LPS responses in vitro and in animal models, providing a molecular explanation to the ARDS seen during COVID-19 (Petruk et al., JMCB, 2020), where an uncontrolled LPS response gives rise to excessive localised inflammation, but also in severe systemic responses to infection. Therefore, although sensing of LPS is important for initial host defence responses, clearance and control of this molecule is critical in order to avoid excessive inflammation and organ damage.

Current treatments based on antibiotics and antivirals target only the microbes and not the accompanying over-activation of immune responses, such as seen in sepsis. This is a leading cause of death in the U.S alone, with over 700,000 cases estimated every year, and with mortality rates from 30-50% in patients with septic shock. Treatment concepts based on Nature's own innate defence strategies, aiming at not only targeting bacteria, but also the excessive immune response, could therefore have a significant therapeutic potential. Thrombin-derived C-terminal peptides (TCP) of roughly 2 kDa have been demonstrated to exert anti-endotoxic functions in vitro and in vivo. Such smaller peptides belong to the diverse family of host-defence peptides (HDPs), which includes neutrophil-derived α-defensins and the cathelicidin LL-37, all known to exhibit immunomodulatory activities. TCP-25 (SEQ ID NO: 12) encompassing sequences of natural TCPs has been shown to neutralise LPS in vitro and protect against P. aeruginosa sepsis and LPS-mediated shock in experimental animal models, mainly via reduction of systemic cytokine responses (Kalle et al., PLOS One, 2011).

TCP-25 binds to LPS and interacts directly with monocytes and macrophages and interferes with CD14 signalling and TLR4/MD2 dimerization thus inhibiting TLR4- and TLR2-induced NF-kB activation in response to microbe-derived agonists and intact bacteria (Saravanan et al., Nat Comm, 2018). TCPs, apart from their interactions with bacterial membranes and LPS, also bind to the LPS-binding groove of CD14 (Saravanan et al., Nat Comm, 2018). The fact that TCPs exert multiple and relatively weak affinities, all in the μM range to LPS and CD14, enables a modulation of host responses to infection. Sharing many characteristics with transient drugs, defined by their multivalency, multiple targets, high-off-rates and K_{d} values at μM levels, TCPs are therefore of interest in the development of novel anti-inflammatory therapies inspired by Nature.

Like many peptide-based therapeutics, TCP-25 is however degraded by endogenous proteases (Puthia et al., 2020). For diseases such as sepsis and ARDS, which require systemic or inhalation applications, a rapidly degraded peptide would require prohibitively large doses and frequent administration. Moreover, from a pharmacological perspective, an improved affinity to its target receptor CD14 is desirable, and would reduce the effective concentration needed. Finally, as TCP-25 forms oligomers and aggregates at higher concentrations, an improved solubility would be an advantage from a drug delivery perspective.

### SUMMARY

The present invention provides peptides with several advantageous properties including one or more of the following:
high in vivo stability
increased stability in the presence of proteases, such as human neutrophil elastase (HNE), Pseudomonas elastase (PE), and/or trypsin
high anti-inflammatory activity, as for example determined by reduced release of inflammatory cytokines, such as e.g. TNF-α and/or IL-1β or reduced NF-κB activity
anti-microbial activity, for example bactericidal activity against Gram negative and/or Gram positive bacteria
low hemolytic activity in blood
low hemolytic activity against RBC
low toxicity

In particular, the peptides of the invention have a low hemolytic activity in blood at a concentration where the peptides have high anti-inflammatory activity.

Preferred peptides of the invention have all of the aforementioned properties. The high in vivo stability, increase stability to proteases as well as the low hemolytic activity render the peptides of the invention particularly useful for systemic administration. In particular, the present invention provides peptides, which have a low hemolytic activity against red blood cells (RBC) at concentrations where they have high anti-inflammatory activity.

More specifically the peptides of the invention are based on thrombin derived peptides, the structure of which have been locked by a covalent linkage between two non-neighbouring amino acids. Interestingly, the peptides of the invention have several—and preferably all—of the aforementioned advantageous properties. Many linear, thrombin derived peptides have both anti-inflammatory and antimicrobial activity, however, in general they have low in vivo stability.

Having a stabilized structure, the peptides of the invention in general comprise helical structure(s). They may have a stabilized protease resistant structure, exert antimicrobial activity, and in general have an improved anti-inflammatory efficacy. The peptides of the invention are therefore interesting lead anti-inflammatory peptide mimetics. The peptides in general have lower tendency to oligomerise compared to native TCPs. Oligomerisation of drugs is a well-known phenomenon, which can cause aggregation, reduce efficacy, and increase the risk for delayed immune reactions. Thus, the peptides of the invention in general show significantly less oligomerization, which is advantageous from a drug perspective.

The endogenous TCP HVF18 exerts a higher affinity to LPS at low pH. Preferred peptides of the invention have increased polarity and charge at the N-terminus, e.g. by additions of cationic K and R residues. Employing a combination of nuclear magnetic resonance spectroscopy (NMR), biophysical, mass spectrometry, microbiological, cellular, and in vivo studies, the invention shows that an increase of charge, and in particular an increase in charge by +2 may yield an optimum efficacy and a high therapeutic index, contrasting to peptides with longer cationic stretches which may be highly toxic and have reduced anti-inflammatory activity.

Stapling of peptides may improve their proteolytic stability, however, surprisingly, stapling of certain thrombin derived peptide also led to undesired effects. For example, stapling of GKY25 at a single position results in a peptide with high hemolytic activity, which is undesirable. Stapling GKY25 at a single position further results in a peptide with reduced anti-inflammatory effect compared to unstapled GKY25.

Surprisingly, the present invention discloses that in contrast to longer peptides, such as GKY25, shorter thrombin derived peptides having a total length of 10 to 23 amino acids, such as 13 to 23 amino acids have most, and often all, of the aforementioned advantageous properties.

Furthermore, the present invention discloses that stapling of longer thrombin derived peptides having a length of 24 to 40 amino acids at at least two positions renders a peptide with some of the aforementioned advantageous properties.

The invention further shows that peptides comprising additional positively charged amino acids have even better anti-inflammatory effect.

Furthermore, the peptides of the invention may also have anti-coagulant activity. The invention shows that peptides comprising additional positively charged amino acids may have even better anti-coagulant activity.

The invention provides peptides comprising a consecutive sequence of in the range of 10 to 23 amino acids from thrombin of SEQ ID NO: 1 containing up to 6 amino acid substitutions, wherein said peptides:
i) have a total length between 10 and 40 amino acids;
ii) comprise at least one internal covalent linkage between the side chains of two non-neighbouring, internal amino acids, wherein said amino acids are denoted X₁ and X₂; and
iii) comprise at least amino acids K247, K248 and K252 of thrombin of SEQ ID NO: 1;
   with the proviso that if the peptide has a total length between 24 to 40 amino acids it comprises at least two internal covalent linkages between the side chains of two non-neighbouring, internal amino acids, wherein the amino acids of the first internal covalent linkage are denoted X₁ and X₂, and the amino acids of the second internal covalent linkage are denoted X₃ and X₄.

### DESCRIPTION OF DRAWINGS

FIG. 1 SDS-PAGE of intact and digested peptides with different proteases for different length of time. One representative image from 3 independent experiments is shown (n=3).

FIG. 2 a NF-κB activation and cell viability in THP1-XBlue-CD14 reporter cells stimulated with 100 ng ml⁻¹ E. coli LPS in the presence or the absence of increasing concentrations of linear and stapled GKY25, 20 h post stimulation. Results are presented as means ±SD of 4 experiments (n=4). Significance was established by an ordinary two-way ANOVA followed by Tukey's multiple comparisons test using GraphPad Prism software. b cytokines released from human blood stimulated with 100 ng ml⁻¹ E. coli LPS in the presence or the absence of increasing concentrations of GKY25 and sGKY25, 24 h post stimulation. Results are presented as mean±SEM. Blood from a different donor was used each time (n=4). Significance was established by an ordinary two-way ANOVA followed by Tukey's multiple comparisons test using GraphPad Prism software.

FIG. 3 Left panels: NF-κB activation and cell viability in THP-1 monocytes stimulated with 100 ng ml⁻¹ E. coli LPS in the presence or the absence of increasing concentrations of HVF18 and sHVF18, 20 h post stimulation. Results are presented as means ±SD of 4 experiments (n=4). Significance was established by an ordinary two-way ANOVA followed by Tukey's multiple comparisons test using GraphPad Prism software. Right panels: cytokines released from human blood stimulated with 100 ng ml⁻¹ E. coli LPS in the presence or the absence of increasing concentrations of HVF18 and sHVF18, 24 h post stimulation. Results are presented as mean±SEM. Blood from a different donor was used each time (n=4). Significance was established by an ordinary two-way ANOVA followed by Tukey's multiple comparisons test using GraphPad Prism software.

FIG. 4 Heatmaps show the hemolytic activity of the peptides on erythrocytes (RBCs) or whole blood. The numbers indicate the % hemolytic activity. Data are presented as mean of 3 independent experiments, each performed with blood from a different donor (n=3). A) GKY25 and sGKY25 and B) HVF18 and sHVF18.

FIG. 5 shows the effects of stapling on anti-inflammatory activity of the peptides. a, Representative binding curves between CD14 and GKY25, HVF18 or sHVF18, in the presence or the absence of NaCl, obtained by MST. Kd were calculated from MST curves. The data are presented as mean±SD of 6 different measurements (n=6). Significance was established by an ordinary one-way ANOVA followed by Tukey's multiple comparisons test using GraphPad Prism software.

FIG. 6 shows the anti-inflammatory activity of linear and stapled HVF18. a, NF-κB activation and cell viability in THP1-XBlue-CD14 reporter cells stimulated with 100 ng ml⁻¹ of E. coli LPS (LPS_{Ec}), 1 μg ml⁻¹ S. aureus LTA (LTA_{Sa}), 1 μg ml⁻¹ E. coli PGN (PGN_{EB}), 1 μg ml⁻¹ S. aureus PGN (PGN_{Sa}), 10 μg ml⁻¹ S. cerevisiae zymosan (Zym_{Sc}) in the presence or the absence of 10 μM of linear and stapled HVF18 20 h post stimulation. Results are presented as means ±SD of 4 experiments (n=4). Significance was established by an ordinary one-way ANOVA followed by Dunnett's multiple comparisons test using GraphPad Prism software.

FIG. 7 shows the anti-inflammatory activity of linear and stapled HVF18 in blood. a, cytokines released from human blood stimulated with 100 ng ml⁻¹ E. coli LPS mixed with increasing doses of HVF18 or sHVF18, 24 h post stimulation. b, cytokines released from human blood stimulated with 100 ng ml⁻¹ E. coli LPS for 30 min and then incubated with increasing doses of HVF18 or sHVF18, 24 h post stimulation Results are presented as mean±SEM. Blood from a different donor was used each time (n=4). Significance was established by an ordinary two-way ANOVA followed by Tukey's multiple comparisons test using GraphPad Prism software.

FIG. 8 shows the effects of stapled peptide on endotoxin responses in experimental mouse models. a, representative in vivo inflammation imaging by IVIS in NF-κB reporter mice. HVF18 or sHVF18 were mixed with LPS immediately before subcutaneous injection on the back of transgenic BALB/c Tg(NF-κB-RE-luc)-Xen reporter mice. In vivo imaging was acquired using an IVIS Spectrum bioimaging system at 3-6-24 hours after subcutaneous deposition. Bar chart shows the measured bioluminescence intensity emitted from these mice. Data are presented as the means ±SEM (n=8 each group). P values were determined using a Mann-Whitney U test. b, cytokine release from plasma collected after 8 and 20 hours from C57BL/6 mice stimulated with sublethal dose of LPS administrated intraperitoneally (i.p.) and then treated with sHVF18 i.p. Data are presented as the means ±SEM (each circle represent a single mouse). P values were determined using ordinary one-way ANOVA following Dunnett's multiple comparisons tests.

FIG. 9 shows the effects of linear and stapled peptide on endotoxin responses in experimental mouse models. Representative in vivo inflammation imaging by IVIS in NF-κB reporter mice. 200 μg HVF18 or sHVF18 were mixed with 25 μg LPS immediately before subcutaneous injection on the back of transgenic BALB/c Tg(NF-κB-RE-luc)-Xen reporter mice. In vivo imaging was acquired using an IVIS Spectrum bioimaging system at 3-6-24 hours after subcutaneous deposition. Bar chart shows the measured bioluminescence intensity emitted from these mice. Data are presented as the means ±SEM (n=5 each group). P values were determined using a Mann-Whitney U test. b, cytokines release from plasma collected after 20 hours from C57BL/6 mice stimulated with sublethal dose of LPS given i.p. and then treated with increasing doses of sHVF18 i.p. Data are presented as the means ±SEM (each circle represent a single mouse). P values were determined using ordinary one-way ANOVA following Dunnett's multiple comparisons tests.

FIG. 10 shows the effects of stapling on antimicrobial activity of HVF18. a, the heatmaps show the antimicrobial activity of increasing concentrations of HVF18 and sHVF18 determined by RDA. The activity was evaluated on E. coli, P. aeruginosa O1, and S. aureus, both in the absence and the presence of NaCl. Data are presented as the zones of clearance. Greyscale and values in each box represent mean values (n=4). b, the killing effect of HVF18 and sHVF18 in Tris buffer, alone or complemented with NaCl or 25% of human plasma, evaluated by VCA. Data are presented as the means ±SEM (n=4). c The killing effect of sHVF18 on S. aureus in Tris buffer evaluated by VCA. Data are presented as the means ±SEM (n=4). d-e, MIC values of HVF18 and sHVF18 for E. coli, P. aeruginosa O1, and S. aureus (e) and clinical isolates for P. aeruginosa O1, and S. aureus (f). Experiment was repeated 4 times with similar results (n=4).

FIG. 11 shows evaluation of secondary structure of sHVF18 and its K and R variants. All peptides were diluted in 10 mM Tris at pH 7.4 at final concentration of 10 μM from 1 mM stock solution. The spectra were acquired at 25° C. Results are presented as the mean of three different experiments.

FIG. 12 shows evaluation of hemolytic property of different stapled peptides in vitro. The histograms show the hemolytic activity of different concentrations of sHVF18 K and R variants on erythrocytes (a) or whole blood (b). Data are the means ±SD of four independent experiments (shown as dots). In (c) the hemolytic activity of GKY25, sGKY25 and 2sGKY25 on whole blood are reported. Data are the means ±SD of two independent experiments. In all graphs dashed line represents the hemolytic activity of 100 μM sHVF18, whereas the dotted line corresponds to 10% of lysis.

FIG. 13 shows evaluation of anti-inflammatory activity of sHVF18 and its K and R variants THP-1-XBlue-CD14 reporter cells. NF-κB activation and cell viability in THP-1 monocytes stimulated with 100 ng ml⁻¹ E. coli LPS in the presence or the absence of increasing concentrations of K (a) and R (b) sHVF18 variants, 20 h post stimulation. Results are presented as means ±SD of 4 experiments (n=4). 10 μM sHVF18 in the presence of LPS was used for comparison.

FIG. 14 shows evaluation of anti-inflammatory activity of stapled peptides in human blood. Cytokines released from human blood stimulated with 100 ng ml⁻¹ E. coli LPS in the presence or the absence of increasing concentrations of K (a) and R (b) sHVF18 variants, or GKY25 and its stapled variants (c), 24 h post stimulation. Results are presented as mean±SEM. Blood from a different donor was used each time (n=4).

FIG. 15 shows evaluation of anti-inflammatory activity of sHVF18 and its K and R variants in mouse endotoxin model. C57BL/6 mice were stimulated with sublethal dose of LPS and after 30 min treated with 10 μg of sHVF18, sKVF18, sKKVF18, sRVF18 or sRRVF18. After 20 h mice were deeply anesthetized by isoflurane and the blood was collected by cardiac puncture. Cytokines release profile is reported as the histograms. Data are presented as the means ±SEM (n=7, a part for sHVF18 and LPS n=13 and untreated n=2). P values were determined using ordinary one-way ANOVA following Dunnett's multiple comparisons tests.

FIG. 16 shows evaluation of antibacterial activity of sHVF18 K and R variants. The heatmaps show the antimicrobial activity of increasing concentrations of the peptides determined by RDA. The activity was evaluated on E. coli, P. aeruginosa O1 (PAO1), and S. aureus, both in the absence (a) and the presence (b) of NaCl. Data are presented as the zones of clearance expressed in mm. Greyscale and values in each box represent mean values (n=4).

FIG. 17 shows evaluation of antibacterial activity of sHVF18 K and R variants in solution. The bactericidal effect of sHVF18 and its variants in 10 mM Tris at pH 7.4, alone (a) or complemented with NaCl (b) evaluated by VCA on E. coli, P. aeruginosa O1 (PAO1), and S. aureus. Data are presented as the means ±SEM (n=4).

FIG. 18 shows the effect of different peptides on coagulation. The activated partial thrombin time (aPTT) and prothrombin time (PT) were determined after addition of increasing concentrations of the different stapled peptides as indicated on the figure to human citrate plasma (n=2).

FIG. 19 shows the hydrodynamic radii (Rh) of different peptides as a measure of oligomerisation. Peptides were resuspended in 10 mM Tris at pH 7.4 or in 10 mM NaOAc at pH 5.0 at 1 mM as the final concentration. 30 μL of each sample were used to measure the hydrodynamic radii (in nm) of particles in solution. For each sample, spectra were recorded three times with 10 sub-runs using the multimodal mode. Each experiment was performed 3 times (n=3). P-values were determined using a one-way ANOVA with Tukey's multiple comparisons test. **P≤0.01, ****P≤0.0001.

FIG. 20 shows a selection of stapled peptide with improved anti-inflammatory activity. a, the heatmaps show the hemolytic activity of the peptides on whole blood or erythrocytes (RBCs). Data from experiments performed on erythrocytes or blood from 4 different donors are presented as mean (n=4). b, the heatmaps show the cytokines released from human blood stimulated with 100 ng ml⁻¹ E. coli LPS in the presence or the absence of increasing concentrations of different peptides, 24 h post stimulation. Results are presented as mean. Blood from a different donor was used each time (n=4). c, graph obtained combining data from (a) and (b), and represents hemolytic activity of the peptides in function of their IC₅₀ for different cytokines as indicated. For peptides with IC₅₀>10 hemolytic activity at 20 μM is shown. d, hemolytic activity of the peptides in function of their IC₅₀ for TNF-α. The graph summarises results obtained as described in Examples 1 and 2. For peptides with IC₅₀>10 μM, where the exact IC₅₀ is unknown, it was chosen to show the hemolytic activity at 50 μM.

FIG. 21 shows the hemolytic and anti-inflammatory activity of K and R variants of sKKW13. a, the heatmaps show the hemolytic activity of the peptides on whole blood. Data from experiments performed on blood from 4 different donors are presented as mean (n=4). b, the heatmaps show the cytokines released from human blood stimulated with 100 ng ml⁻¹ E. coli LPS in the presence or the absence of increasing concentrations of different peptides, 24 h post stimulation. Results are presented as mean. Blood from a different donor was used each time (n=4).

FIG. 22 shows intravenous administration of sHVF18 ameliorated septic like conditions and hindered development of severe states of ARDS in pigs. The figure shows results from pigs with acute lung injury and ARDS treated with and without sHVF18. (a) Figure shows an overview of the experiment setup. Pigs were anesthetized in mechanical ventilation and monitored continuously using an arterial line and swan-Ganz catheter. LPS was given intravenously and treated animals received sHVF18. Hemodynamics, vitals, and pulmonary gas exchange were followed continuously over the time course of the experiment. (b) shows pulmonary gas exchange as PaO₂ FiO₂⁻¹ ratio between treated (n=5) and not treated pigs (n=5). All non-treated animals developed mild to moderate ARDS. A significant increase in cardiac output (c), a significant decrease in urine output (d), and a significant increased need of inotropic support (e), and a significant increase in lactate levels (f) was seen in the non-treated animals but not in the treated animals indicating a severe stage of septic like condition. (g) Images representative of n=15 samples of hematoxylin and eosin (H&E) histology of healthy controls (n=5) (left), non-treated (middle) (n=5) and sHVF18-treated (right) (n=5) lungs. Scale bar in the larger image represents 0.5 mm. The callout shows a magnified portion of the tissue where the scale bar represents 0.2 mm. (h) Results of cumulative blinded scoring of the histology. Statistically significant differences between non-treated and treated groups were tested with two-sided Student's T-test and within groups with 2-sided ANOVA. *p<0.05, **p<0.01, ***p<0.001. All values represent the mean±standard deviation.

FIG. 23 shows in silico analysis of staple positions. (a) HVF18 was docked on CD14 and the N-terminal GKYGFYT residues were modelled to form GKY25 peptide. The binding energy of GKY25 to CD14 was calculated using MMPBSA. Indicated amino acids were substituted with pentenyl alanine, and a staple was added to connect residues i and i+3 along the sequence of the peptide. MMPBSA was used to calculate the binding energy of the stapled peptide to CD14. The graph shows the binding energy difference between non-stapled and stapled GKY25 for all staple positions. Positive values indicate worse binding, while negative values indicate more favorable binding for the stapled peptide. (b) A similar analysis was performed by adding the staple to connect residues i and i+4. (c and d) A similar analysis was also performed for the shorter HVF18 peptide.

### DETAILED DESCRIPTION

### Definitions

The term “amino acid” as used herein refers to any amino acid, such as any canonical and non-canonical amino acid.

The term “canonical amino acid” as used herein refers to a proteinogenic amino acid. Preferably, the proteinogenic amino acid is one of the 20 amino acids encoded by the standard genetic code. The IUPAC one and three letter codes are used to name amino acids.

The term “covalent linkage” between two side chains of amino acids as used herein refers to either a covalent bond between said side chains or to that said side chains are bound covalently to each end of a linker, so that all bonds connecting the side chains are covalent.

The term “hydrocarbon staple” as used herein refer to an alkyl or alkenyl moiety linking to amino acid side chains. Typically, the “hydrocarbon staple” is a C₆₋₁₆ alkenyl moiety comprising one or more double bonds.

The term “internal” as used herein in relation to amino acids within a peptide, refers to that the amino acids is neither not positioned as the most N-terminal nor as the most C-terminal amino acids in the primary sequence of the peptide.

The term “non-neighbouring” as used herein in relation to amino acids within a peptide, refers to that two amino acids are not positioned next to each other in the primary sequence of the peptide.

The term “position n” as used herein in relation to amino acids within a peptide refers to position in the primary sequence, wherein the most N-terminal amino acids has position n.

The term “stapled peptide” as used herein refer to a peptide comprising at least one covalent linkage between the side chains of two non-neighbouring, internal amino acids. In particular, a stapled peptide may comprise a hydrocarbon staple.

### Peptide Comprising Internal Covalent Linkage

The present invention provides peptides comprising a consecutive sequence of in the range of 10 to 23 amino acids from thrombin of SEQ ID NO: 1 containing up to 6 amino acid substitutions, wherein said peptides:
i) have a total length between 10 and 40 amino acids, preferably a total length between 10 and 23, more preferably a total length between 13 and 23;
ii) comprise at least one internal covalent linkage between the side chains of two non-neighbouring, internal amino acids, wherein said amino acids are denoted X₁ and X₂; and
iii) comprise at least amino acids K247, K248 and K252 of thrombin of SEQ ID NO:1;
   with the proviso that if the peptide has a total length between 24 to 40 amino acids it comprises at least two internal covalent linkages between the side chains of two non-neighbouring, internal amino acids, wherein the amino acids of the first internal covalent linkage are denoted X₁ and X₂, and the amino acids of the second internal covalent linkage are denoted X₃ and X₄.

The present invention further provides peptides comprising a consecutive sequence of in the range of 10 to 40 amino acids from thrombin of SEQ ID NO: 1 containing up to 6 amino acid substitutions, wherein said peptides:
i) have a total length between 10 and 40 amino acids, preferably a total length between 10 and 23, more preferably a total length between 13 and 23;
ii) comprise at least one internal covalent linkage between the side chains of two non-neighbouring, internal amino acids, wherein said amino acids are denoted X₁ and X₂; and
iii) comprise at least amino acids K247, K248 and K252 of thrombin of SEQ ID NO: 1;
   with the proviso that if the peptide has a total length between 24 to 40 amino acids it comprises at least two internal covalent linkages between the side chains of two non-neighbouring, internal amino acids, wherein the amino acids of the first internal covalent linkage are denoted X₁ and X₂, and the amino acids of the second internal covalent linkage are denoted X₃ and X₄.

The invention further provides peptides comprising or even consisting of a consecutive sequence of amino acids from thrombin of SEQ ID NO: 1 containing up to 6 amino acid substitutions, wherein said peptide:
i) has a total length between 10 and 23 amino acids;
ii) comprises at least one covalent linkage between the side chains of two non-neighbouring, internal amino acids, wherein said amino acids are denoted X₁ and X₂ herein; and
iii) comprises at least amino acids K247, K248, K252 of thrombin of SEQ ID NO: 1.

The invention further provides peptides comprising or even consisting of a consecutive sequence of amino acids from thrombin of SEQ ID NO: 1 containing up to 6 amino acid substitutions, wherein said peptide:
i) has a total length between 13 and 23 amino acids;
ii) comprises at least one covalent linkage between the side chains of two non-neighbouring, internal amino acids, wherein said amino acids are denoted X₁ and X₂ herein; and
iii) comprises at least amino acids K247, K248, K252 of thrombin of SEQ ID NO: 1.

In some embodiments, the peptides of the invention comprise at least amino acids R245, K247, K248 and K252 of thrombin of SEQ ID NO: 1

The internal covalent linkage may be as described herein below in the section “Internal covalent linkage”.

Preferably, the peptide has one or more of the advantageous properties described in the “Summary” herein above or the section “Peptide Function” below.

The peptides are useful for treatment of inflammation and/or infection, e.g. as described in the section “Method of treatment”.

The sequence of prothrombin is given herein as SEQ ID NO: 16. Prothrombin may be cleaved at Arg²⁷¹. This cleavage produces two fragments known as Fragment 1⋅2, comprising the first 271 residues of prothrombin and the intermediate prethrombin 2, which is made up of residues 272-579. Fragment 1⋅2 is released as an activation peptide, and prethrombin 2 is cleaved at Arg³20, yielding active thrombin. The sequence of active thrombin is given herein as SEQ ID NO: 1.

### Internal Covalent Linkage

One hallmark of the peptides of the invention is that they contain a covalent linkage between the side chains of two non-neighbouring, internal amino acids. Thus, said covalent linkage is either a direct covalent bond between the side chains of said amino acids or the side chains are linked covalently to each other through a linker. In other words, covalent linkages in the peptide back-bone are not considered “a covalent linkage between the side chains of two non-neighbouring, internal amino acids” according to the invention.

### Shorter Peptides

Whereas it is possible that the peptide contains more than one such covalent linkage, it is preferred that the peptide contains only one covalent linkage between two non-neighbouring, internal amino acids if said peptide is between 10 and 23 amino acids. The amino acids having an internal linkage between the side chains are also denoted X₁ and X₂ herein.

Whereas X₁ and X₂ are bound to each other by a covalent linkage in the peptides of the invention, X₁ and X₂ may be described in their free, unbound form herein. The skilled person will understand that even if X₁ and X₂ are described in their unbound form, in the final peptides of the invention, they will have formed the relevant covalent linkage. By way of example, X₁ and X₂ may each be described as “(S)-2-(4′-pentenyl)-alanines”, however in the peptide of the invention, the pentenyl groups will have reacted—typically by ring closing metathesis—to form a linker consisting of an 8 carbon long alkenyl with one double bond only.

Peptides comprising a covalent linkage between two non-neighbouring, internal amino acids are also known as “stapled” peptides.

There are many different ways of forming stapled peptides known to the skilled person and the peptides of the invention may comprise any kind of covalent linkage between two non-neighbouring, internal amino acids useful for peptide stapling. For example, the peptide may comprise any of the staples described in U et al, 2020 or in international patent application WO02019018499 both of which are incorporated herein by reference in their entirety.

It is preferred that when amino acid X₁ is positioned at position n, then amino acid X₂ is positioned at position n+3, or at position, n+4, or at position n+5, or at position n+6, or at position n+7, or at position n+8, or at position n+9, or at position n+10, or at position n+11, wherein n is an integer. More preferably, when amino acid X₁ is positioned at position n, then amino acid X₂ is positioned at position n+3, or at position n+4, or at position n+7, or at position n+11, wherein n is an integer. Even more preferably, when amino acid X₁ is positioned at position n, then amino acid X₂ is positioned at position at position n+4, or at position n+7, wherein n is an integer. The latter positioning pattern is especially useful for supporting an α-helical structure of the peptide. Typically, n is an integer in the range of 2 to 18, however n must be chosen such that X₁ is not positioned at the very N-terminus, more preferably, neither X₁ nor X₂ is positioned at the very N-terminus or the very C-terminus.

In principle, X₁ can be positioned at any position within the peptide apart from at the very N-terminus. However, certain positions within the peptide may be more favourable than others. The peptides of the invention comprises a consecutive sequence of amino acids from Thrombin of SEQ ID NO: 1 or from GKY25 of SEQ ID NO: 12. In the following, the position of the amino acids is given in relation to the amino acid numbering of GKY25 of SEQ ID NO: 12. Thus, any amino acid having the same position as a given amino acid in GKY25 of SEQ ID NO: 12 following an alignment, is referred to as “aligning to” said amino acid of GKY25.

In one embodiment it is preferred that following alignment of the sequence of the peptide of the invention to the sequence of GKY25 of SEQ ID NO: 12, then
a. X₂ does not align to a Lys in GKY25 of SEQ ID NO: 12; and
b. X₂ does not align to a Gln, when X₁ aligns to a Lys.

In one embodiment it is preferred that following alignment of the sequence of the peptide of the invention to the sequence of GKY25 of SEQ ID NO: 12, then
i) X₁ does not align to Arg11 in GKY25 of SEQ ID NO: 12
ii) X₁ does not align to Lys14 in GKY25 of SEQ ID NO: 12
iii) X₂ does not align to Lys14 in GKY25 of SEQ ID NO: 12; and
iv) X₂ does not align to Lys 18 in GKY25 of SEQ ID NO: 12; and
v) X₂ does not align to Gln22, when X₁ aligns to Lys18 of SEQ ID NO: 12.

In one embodiment, amino acid X₁ is positioned at position n and amino acid X₂ is positioned at position at position n+3, wherein n is an integer in the range of 2 to 18, and following alignment of the sequence of the peptide of the invention to the sequence of GKY25 of SEQ ID NO: 12, then
i) X₁ does not align to Arg11 in GKY25 of SEQ ID NO: 12
ii) X₁ does not align to Lys14 in GKY25 of SEQ ID NO: 12; and
iii) X₂ does not align to Lys14 in GKY25 of SEQ ID NO: 12.

In one embodiment, amino acid X₁ is positioned at position n and amino acid X₂ is positioned at position at position n+4, wherein n is an integer in the range of 2 to 18, and following alignment of the sequence of the peptide of the invention to the sequence of GKY25 of SEQ ID NO: 12, then
i) X₁ does not align to Arg11 in GKY25 of SEQ ID NO: 12
ii) X₁ does not align to Leu12 in GKY25 of SEQ ID NO: 12
iii) X₁ does not align to Lys14 in GKY25 of SEQ ID NO: 12
iv) X₂ does not align to Lys14 in GKY25 of SEQ ID NO: 12;
v) X₁ does not align to Lys 18 in GKY25 of SEQ ID NO: 12; and
vi) X₂ does not align to Lys 18 in GKY25 of SEQ ID NO: 12.

In a preferred embodiment, amino acid X₁ is positioned at position n and amino acid X₂ is positioned at position at position n+3, wherein n is an integer in the range of 2 to 18, and wherein when aligning the sequence of the peptide of the invention to the sequence of GKY25 of SEQ ID NO: 12, then X₁ and X₂ corresponds to
Val9 and Leu12; or
Phe10 and Lys13; or
Leu12 and Trp15; or
Lys13 and Ile16; or
Ile16 and Val19; or
Gln17 and Ile20; or
Lys18 and Asp21; or
Val19 and Gln22; or
Ile20 and Phe23; or
Asp21 and Gly24; or
Gln22 and Glu25
of SEQ ID NO: 12.

In another preferred embodiment, amino acid X₁ is positioned at position n and amino acid X₂ is positioned at position at position n+4, wherein n is an integer in the range of 2 to 18, wherein when aligning the sequence of the peptide of the invention to the sequence of GKY25 of SEQ ID NO: 12, then X₁ and X₂ corresponds to
Val9 and Lys13; or
Lys13 and Gln17; or
Trp15 and Val19; or
Ile16 and Ile20; or
Gln17 and Asp21; or
Val19 and Phe23; or
Ile20 and Gly24; or
Asp21 and Glu18
of SEQ ID NO: 12.

In a very preferred embodiment, amino acid X₁ is positioned at position n and amino acid X₂ is positioned at position at position n+4, wherein n is an integer in the range of 2 to 18, wherein when aligning the sequence of the peptide of the invention to the sequence of GKY25 of SEQ ID NO: 12, then X₁ and X₂ corresponds Gln17 and Asp21, respectively.

In one embodiment, X₁ and X₂ are canonical amino acids before reaction to form the covalent linkage. For example, X₁ and X₂ may before reaction to form the covalent linkage be selected from the group consisting of:
i) X₁ is Lys and X₂ is selected from the group consisting of Asp, Glu, Lys, Cys and Tyr;
ii) X₁ is Cys and X₂ is selected from the group consisting of Cys, Lys and Met;
iii) X₁ is Asp and X₂ is Lys;
iv) X₁ is Glu is X₂ is selected from the group consisting of Lys and Glu;
v) X₁ is Tyr and X₂ is selected from the group consisting of Lys, Phe and Trp;
vi) X₁ is Met and X₂ is selected from the group consisting of Met and Cys;
vii) X₁ is His and X₂ is His;
viii) X₁ is Phe and X₂ is selected from the group consisting of Phe, Tyr, Ala and Trp;
ix) X₁ is Ala and X₂ is Phe or Tyr;
x) X₁ is Trp and X₂ is selected from the group consisting of Trp, Phe and Tyr.

In one embodiment, X₁ and X₂ may before reaction to form the covalent linkage be as follows:
X₁ is Lys and X₂ is Asp, Glu, Cys or Lys or vice versa.

X₁ and X₂ are Cys.

The covalent linkage may be formed by a direct reaction between the side chains of the canonical amino acids, and it may be formed via a cross linker. When X₁ and X₂ are Cys the covalent linkage may be a disulphide bridge or it may be formed via a crosslinker, wherein the crosslinker for example is a bis-alkylator, such as linker comprising at least two (bromomethyl) substituents.

In a preferred embodiment, X₁ and X₂ are derivatised canonical amino acids. Before reaction to form the covalent linkage, X₁ and/or X₂ may for example be selected from the group consisting of Ser derivatives and Ala derivatives.

In one embodiment, the covalent linkage is formed by linking two non-canonical amino acids. For example, the covalent linkage may be formed by linking two non-canonical amino acids, which have substituted two native amino acids of the consecutive sequence from thrombin.

In one embodiment the covalent linkage is a hydrocarbon staple.

In one preferred embodiment, X₁ and X₂ before reaction to form the covalent linkage are alkenylated amino acids, such as two C-alkenylated amino acids, such as two α-substituted alkenyl amino acids and/or α,α-disubstituted alkenyl amino acids, and the covalent linkage is an olefin tether formed between said alkenyl residues.

Said alkenylated amino acids may be amino acids native to thrombin, which have been alkenylated. Alternatively, said alkenylated amino acids may be amino acids substituting amino acids native to thrombin.

In one preferred embodiment, X₁ and X₂ before reaction to form the covalent linkage may individually be selected from the group consisting of alkenylated Ala, alkenylated Leu, alkenylated Met, alkenylated Ser, alkenylated Tyr, alkenylated Lys, alkenylated Arg and alkenylated Phe. In such cases the covalent linkage is an olefin tether formed between said alkenyl residues.

In one preferred embodiment, one of X₁ and X₂ before reaction to form the covalent linkage may be alkenylated Ala and the other may be selected from the group consisting of alkenylated Ala, alkenylated Leu, alkenylated Met, alkenylated Ser, alkenylated Tyr, alkenylated Lys, alkenylated Arg and alkenylated Phe. In such cases the covalent linkage is an olefin tether formed between said alkenyl residues.

In one preferred embodiment, wherein X₁ and X₂ are α-alkenyl olefin-terminated amino acids and/or α,α-disubstituted alkenyl olefin-terminated amino acids.

In one preferred embodiment, X₁ and X₂ before reaction to form the covalent linkage may be alkenylated alanine, preferably α-substituted alkenyl or α,α-disubstituted alkenylated alanine In such cases the covalent linkage is an olefin tether formed between said alkenyl residues.

In one preferred embodiment, X₁ and X₂ before reaction to form the covalent linkage may be alkenylated Ser, such as O-alkenylated Ser.

Said alkenylated amino acids comprise 2 to 10 carbons in the alkenyl chain, such as 2, 3, 4, 5, 6, 7, 8, 9, or 10 carbons, preferably 4, 5 or 6 carbons. Said alkenylated amino acids may contain one or more double bonds, however preferably only one double bond. It is further preferred that the double bond is positioned at the free end of the alkenyl. Two double bonds positioned at the free end of two alkenyl residues can react by RCM to form an olefin tether.

Thus, it is preferred that X₁ and X₂ are amino acids, which are linked by an olefin tether formed between said alkenyl residues. Said olefin tether may be a C₆₋₁₆ alkenyl, such as a C₈₋₁₄ alkenyl, for example a C₈ or a C₁₄ alkenyl tether. Said tether may contain one or more double bonds, preferably one double bond.

In one preferred embodiment, X₁ and X₂ before reaction to form the covalent linkage may be α,α-disubstituted S- or R-pentenylalanine (S5 or R5) or S- or R-octenylalanine (S8 or R8) alanine. Thus, the internal hydrocarbon staple may be formed by linking two α,α-disubstituted S- or R-pentenylalanine (S5 or R5) or S- or R-octenylalanine (S8 or R8) alanine.

In one preferred embodiment, X₁ and X₂ before reaction to form the covalent linkage may be (S)-2-(4′-pentenyl)-alanines. Thus, the internal hydrocarbon staple may be formed by linking two (S)-2-(4′-pentenyl)-alanines.

In one embodiment the covalent bond is established through ring-closing, such as through ring closing metathesis (RCM).

In one preferred embodiment, one of X₁ and X₂ before reaction to form the covalent linkage may non-canonical azido terminated amino acid and the other a non-canonical yne-terminated amino acid.

### Longer Peptides

It is preferred that peptides of the invention which are 24 amino acids or longer comprise at least two staples. In other words, it is preferred that peptides of the invention which are 24 amino acids or longer, such as between 24 and 40 amino acids, comprise at least two internal covalent linkages between the side chains of two non-neighbouring, internal amino acids, wherein the amino acids of the first internal covalent linkage are denoted X₁ and X₂, and are as described above, and the amino acids of the second internal covalent linkage are denoted X₃ and X₄.

It is also preferred that peptides comprising the 4 most N-terminal amino acids of GKY25 of SEQ ID NO: 12 also comprises at least two staples, wherein the amino acids of the first internal covalent linkage are denoted X₁ and X₂, and are as described above, and the amino acids of the second internal covalent linkage are denoted X₃ and X₄.

X₃ and X₄ may be as described above in the section “Shorter peptides”. Preferably, X₃ and X₄ are closer to the N-terminal as compared to X₁ and X₂. In particular, X₃ and X₄ may be positioned so that X₃ is positioned at one of the first 4 amino acids. If the peptide comprises the 4 most N-terminal amino acids of GKY25 of SEQ ID NO: 12, it is preferred that X₃ is positioned at a position corresponding to one of the 4 most N-terminal amino acids of GKY25 of SEQ ID NO: 12.

In some embodiments, amino acid X₃ is positioned at position n, and amino acid X₄ is positioned at position n+3, or at position, n+4, or at position n+5, wherein n is an integer. In preferred embodiments, amino acid X₃ is positioned at position n, and amino acid X₄ is positioned at position, n+4, wherein n is an integer in the range of 1 to 18, preferably in the range of 1 to 10, more preferably in the range of 1 to 5. Thus, whereas it is preferred that X₁ is not positioned at the very N-terminus, X₃ may very well be positioned at the very N-terminus.

If X₃ is positioned at the very N-terminus, the staple may be formed between the N-terminal —NH₂ group and the side chain of an amino acid, preferably with the side chain of a Glu or an Asp, more preferably a Glu.

In certain embodiments, amino acid X₃ is positioned at the very N-terminus of the peptide. In preferred embodiments, amino acid X₃ is positioned at the very N-terminus and amino acid X₄ is positioned at position n+4. In very preferred embodiments, when aligning the sequence of the peptide of the invention to the sequence of GKY25 of SEQ ID NO: 12, then X₃ and X₄ corresponds to Gly1 and Phe5 of GKY25 of SEQ ID NO: 12, wherein Phe5 is substituted with either Glu or Asp, preferably Glu. The latter positioning pattern of X₃ and X₄ is especially useful for protecting or masking protease site(s) of the peptide.

The covalent linkage of X₃ and X₄ may for example be an amide bond formed between a carboxylic acid moiety and an amine. In some embodiments, the carboxylic acid moiety is a glutamic acid side chain. In some embodiments, the amine is an amino acid side chain. In other embodiments, the amine is an N-terminal amine group of a peptide backbone. In some embodiments, the covalent linkage of X₃ and X₄ is a lactam bridge formed between an N-terminal amine group and a side chain carboxylic acid of said respective amino acids.

In preferred embodiments, Phe5 of GKY25 of SEQ ID NO: 12 is substituted by Glu5. In such embodiments, X₃ and X₄ corresponds to Gly1 and Glu5 of SEQ ID NO: 14.

### Peptide Properties

The peptides of the invention may have one or more of the following properties.

The invention shows that stapled, longer thrombin derived peptides, such as GKY25, containing a single staple have several less desirable properties. Thus, GKY25 containing one staple has high hemolytic activity and low anti-inflammatory activity in blood, which therefore precludes in vivo use.

In contrast, stapled shorter thrombin derived peptides, such as stapled HVF18 have relatively low hemolytic activity in blood and high anti-inflammatory activity in blood.

Furthermore, double-stapled longer thrombin derived peptides, such as GKY25 with two staples, have lower hemolytic activity in blood compared to single stapled GKY25, and higher stability compared to non-stapled GKY25.

### Shorter Peptides

Accordingly, it is preferred that the peptide of the invention has an appropriate length. Thus, preferably the peptide of the invention has a length between 10 and 23 amino acids, such as between 13 and 18 amino acids, such as between 12 and 22 amino acids, such as between 14 and 22 amino acids, such as between 15 and 21 amino acids, such as between 16 and 20 amino acids, such as between 17 and 20 amino acids.

In particular, the peptide may have a length of 18 to 20 amino acids, such as 18 or 19 amino acids.

The length of the peptide indicates the total length of the peptide. Thus, even if the peptide may be conjugated to one or more additional moieties, the peptide preferably does not comprise more than the indicated number of amino acids.

Furthermore, it is also preferred that the peptide of the invention comprises a consecutive sequence of amino acids from thrombin of SEQ ID NO: 1, which has an appropriate length. Thus, preferably the peptide of the invention comprises or consists of a consecutive sequence of amino acids from thrombin of SEQ ID NO: 1 of in the range of 10 to 23 amino acids, such as between 13 and 23 amino acids, such as between 13 and 18 amino acids, such as between 14 and 22 amino acids, such as between 15 and 21 amino acids, such as between 16 and 20 amino acids, such as between 17 and 20 amino acids, preferably between 17 and 18 amino acids, more preferably between 18 and 19 amino acids.

The peptide according to the invention comprises or even consists of a consecutive sequence of amino acids from thrombin of SEQ ID NO: 1, wherein up to 6 amino acids may be exchanged. In other words, the peptide may comprise or consist of a consecutive sequence of amino acids from thrombin of SEQ ID NO: 1, however from 1 to 6 of the amino acids within said consecutive sequence may be substituted for another amino acid. It is however important that the peptide comprises at least amino acids K247, K248, K252 of thrombin of SEQ ID NO: 1. In other words, said amino acids should not be substituted.

In some embodiments, the peptide comprises at least amino acids R245, K247, K248 and K252 of thrombin of SEQ ID NO: 1.

The peptide according to the invention may in particular have a total length between 10 and 23 amino acids and comprise or even consist of a consecutive sequence of amino acids from GKY25 of SEQ ID NO: 12, wherein up to 6 amino acids may be exchanged. In other words, the peptide may comprise or consist of a consecutive sequence of amino acids from GKY25 of SEQ ID NO: 12, however from 1 to 6 of the amino acids within said consecutive sequence may be substituted for another amino acid. It is however important that the peptide comprises at least amino acids K13, K14 and K18 of GKY25 of SEQ ID NO: 12. In other words, said amino acids should not be substituted.

In some embodiments, the peptide comprises at least amino acids R11, K13, K14 and K18 of GKY25 of SEQ ID NO: 12.

The peptide according to the invention may preferably comprise or even consist of a consecutive sequence of amino acids from thrombin of SEQ ID NO: 1 of in the range of 16 to 21 amino acids, more preferably of in the range of 17 to 18 amino acids.

The consecutive sequence may contain up to 6 amino acid substitutions. For example, said peptide may comprise at least 2 amino acid substitutions, such as 3 amino acid substitutions, such as 4 amino acid substitutions, such as 5 amino acids substitutions compared to the consecutive sequence of thrombin. Preferably, the consecutive sequence may comprise up to 4 amino acid substitutions, even more preferably the consecutive sequence may comprise up to 2 amino acid substitutions. One or more of said substitutions may be conservative substitutions, for example 1, 2, 3 or 4 amino acid substitutions may be conservative substitutions.

In a very preferred embodiment, the peptides comprise or even consist of a consecutive sequence of amino acids from thrombin of SEQ ID NO: 1, wherein one amino acid has been substituted with amino acid X₁ (e.g. any of X₁ described in the section “Internal covalent linkage”) and another amino acid has been substituted with X₂ (e.g. any of X₂ described in the section “Internal covalent linkage”). Said amino acids, which are substituted are preferably positioned in relation to each other as described for amino acids X₁ and X₂ in the section “Internal covalent linkage”.

In addition, to the consecutive sequence of SEQ ID NO: 1, the peptides of the invention may comprise one or more additional amino acids. Preferably, the peptide may contain up to 4 additional amino acids, for example up to 3 additional amino acids, such as 2 additional amino acids. Said additional amino acids may for example be any of the amino acids described in the section “Positively charged amino acids” below.

In one embodiment, the peptide according to the invention comprises or consists of a consecutive sequence of amino acids from thrombin of SEQ ID NO: 1 of in the range of 17 to 18 amino acids containing up to 2 amino acid substitutions, where the peptide may comprise up to 4 additional amino acids.

In one embodiment, the peptide according to the invention comprises or consists of a consecutive sequence of amino acids from thrombin of SEQ ID NO: 1 of in the range of 17 to 18 amino acids containing a substitution of one amino acid for amino acid X₁ and a substitution of one amino acid for amino acid X₂, wherein the peptide may comprise up to 4 additional amino acids.

Thus, in one embodiment, the peptide of the invention consists of in the range of 15 to 20 consecutive amino acids of thrombin of SEQ ID NO: 1, wherein 2 amino acids has been substituted with X₁ and X₂, wherein X₁ and X₂ are alkenylated amino acids forming an internal hydrocarbon staple, and in the range of 2 to 5, preferably 2 additional N-terminal amino acids.

In one embodiment, the peptide of the invention consists of in the range of 16 to 18 consecutive amino acids of thrombin of SEQ ID NO: 1, wherein 2 amino acids has been substituted with X₁ and X₂, wherein X₁ and X₂ are alkenylated amino acids forming an internal hydrocarbon staple, and in the range of 2 to 5 additional N-terminal amino acids, preferably 2 additional N-terminal amino acids.

In one embodiment, the peptide of the invention consists of 17 consecutive amino acids of thrombin of SEQ ID NO: 1, wherein 2 amino acids have been substituted with X₁ and X₂, wherein X₁ and X₂ are alkenylated amino acids forming an internal hydrocarbon staple, and in the range of 2 to 3 additional N-terminal amino acids.

Said additional N-terminal amino acids are preferably positively charged amino acids, for example Lys or Arg.

The peptide of the invention may further comprise one or more moieties conjugated to said peptide. Said moieties may optionally be linked to the peptide via a linker. Said one or more conjugated moieties may for example be selected from the group consisting of alkyls, aryls, heteroaryls, olefins, fatty acids, polyethylene glycol (PEG), saccharides, and polysaccharides.

### Longer Peptides

However, the peptide of the invention may also be longer. In some embodiments, the peptide of the invention has a length between 24 and 40 amino acids, such as between 25 and 35 amino acids, such as between 25 and 30 amino acids, such as between 28 and 34 amino acids.

In particular, the peptide may have a length of 24 to 28 amino acids, such as 25 or 26 amino acids.

Peptides of the invention which are 24 amino acids or longer comprise at least two staples.

Peptides of the invention with a length of 24 to 40 amino acids comprise a consecutive sequence of amino acids from thrombin of SEQ ID NO: 1. Thus, preferably, longer peptides of the invention comprises or consists of a consecutive sequence of amino acids from thrombin of SEQ ID NO: 1 of in the range of 24 to 40 amino acids, such as between 25 and 35 amino acids, such as between 25 and 30 amino acids, such as between 28 and 34 amino acids, preferably between 24 and 28 amino acids, even more preferably between 25 and 26 amino acids.

Longer peptides of the invention may be as defined above in the section “Peptide properties—Shorter peptides”.

Thus, longer peptides according to the invention comprises or even consists of a consecutive sequence of amino acids from thrombin of SEQ ID NO: 1, wherein up to 6 amino acids may be exchanged. It is however important that the peptide comprises at least amino acids K247, K248 and K252 of thrombin of SEQ ID NO: 1. In other words, said amino acids should not be substituted. In some embodiments, the longer peptide according to the invention comprises R245, K247, K248 and K252 of thrombin of SEQ ID NO: 1.

In a very preferred embodiment, the longer peptides of the invention comprise or even consist of a consecutive sequence of amino acids from thrombin of SEQ ID NO: 1, wherein one amino acid has been substituted with amino acid X₁ (e.g. any of X₁ described in the section “Internal covalent linkage”) and another amino acid has been substituted with X₂ (e.g. any of X₂ described in the section “Internal covalent linkage”), and another amino acid has been substituted with X₃ (e.g. any of X₃ described in the section “Internal covalent linkage”), and another amino acid has been substituted with X₄ (e.g. any of X₄ described in the section “Internal covalent linkage”). Said amino acids, which are substituted are preferably positioned in relation to each other as described for amino acids X₁ and X₂, and X₃ and X₄, in the section “Internal covalent linkage”.

### Positively Charged Amino Acids

Interestingly, the present invention shows that insertion of one or more positively charged amino acids may significantly enhance the anti-inflammatory effect of the peptides.

Thus, peptide according to the invention may in preferred embodiments comprise between 1 and 5, such as between 1 and 4, for example between 1 and 3, for example between 1 and 2, such as 2 positively charged amino acids inserted at or close to the end of the peptide.

In particular, said positively charged amino acids may be inserted at or close to the N-terminal, such as at a position selected from positions 1, 2, 3, 4 and/or 5 relative to the N-terminal of the peptide.

In a preferred embodiment, the positively charged amino acids are inserted at the N-terminal.

In one embodiment, the peptide of the invention comprises 2 positively charged amino acids inserted at or close to the N-terminal, such as at a position selected from positions 1, 2, and/or 3 relative to the N-terminal of the peptide.

Said positively charged amino acids may preferably be selected form the group consisting of arginine, lysine and histidine. Preferably, the positively charged amino acids are arginine and/or lysine, even more preferably wherein the positively charged amino acids are lysine.

### Peptide Sequence

In some embodiments of the invention, the peptide may have one of the peptide sequences described herein in this section. In addition to the peptide having the sequences described in this section, it is preferred that the peptide also:
comprises a consecutive sequence of in the range of 10 to 23 amino acids, preferably in the range of 13 to 23 amino acids from thrombin of SEQ ID NO: 1
has a total length of between 10 to 40 amino acids
comprises at least amino acids K247, K248 and K252 of thrombin of SEQ ID NO: 1, more preferably comprises at least amino acids R245, K247, K248 and K252 of thrombin of SEQ ID NO: 1;
   with the proviso that if the peptide has a total length between 24 to 40 amino acids it comprises at least two internal covalent linkages between the side chains of two non-neighbouring, internal amino acids.

### Shorter Peptides

In some embodiments of the invention, the peptide may have one of the peptide sequences described herein in this section. In addition to the peptide having the sequences described in this section, it is preferred that the peptide also:
comprises a consecutive sequence of in the range of 10 to 23 amino acids, preferably in the range of 13 to 23 amino acids from thrombin of SEQ ID NO: 1
has a total length of between 10 to 23 amino acids, preferably between 13 and 23 amino acids
comprises at least amino acids K247, K248 and K252 of thrombin of SEQ ID NO: 1, more preferably comprises at least amino acids R245, K247, K248 and K252 of thrombin of SEQ ID NO: 1.

In some embodiments of the invention, the peptide may have one of the peptide sequences described herein in this section. In addition to the peptide having the sequences described in this section, it is preferred that the peptide also:
comprises a consecutive sequence of in the range of 10 to 23 amino acids, preferably in the range of 13 to 23 amino acids from GKY25 of SEQ ID NO: 12
has a total length of between 10 to 23 amino acids, preferably between 13 and 23 amino acids
comprises at least amino acids K13, K14 and K18 of GKY25 of SEQ ID NO: 12, more preferably comprises at least amino acids R11, K13, K14 and K18 of GKY25 of SEQ ID NO: 12.

X₁ and X₂ in the sequences of this section may for example be as described herein above in the section “Internal covalent linkage”.

U in the sequences of this section may for example be His, Arg or Lys, preferably Arg or Lys.

Z in the sequences of this section may individually be any canonical amino acid. In preferred embodiments most or even all Z of a sequence are selected to correspond to the amino acids of GKY25 of SEQ ID NO: 12.

In one embodiment, the peptide according to the invention comprises the sequence:

-K-K-Z-Z-X₁-K-Z-Z-X₂-Z
wherein
Z is any canonical amino acid; and
X₁ and X₂ are amino acids, the side chains of which are linked by a covalent linkage.

In one embodiment, the peptide according to the invention comprises or consists of the amino acid sequence:

-U-U-(Z)ₙ-I-Q-K-V-I-D-Q-(Z)ₘ-
wherein
the peptide has a total length between 10 to 23 amino acids; and
each Z is individually any canonical amino acid; and
U is His, Lys or Arg; and
n is an integer in the range of 0 to 10; and
m is an integer in the range of 0 to 5, and
wherein two of the amino acids have been substituted for alkenylated amino acids, the side chains of which are linked by a covalent linkage.

In one embodiment, the peptide of the invention comprises or consists of the sequence:

U-U-(Z)ₙ-K-K-Z-Z-X₁-K-Z-Z-X₂-Z,

or the sequence: U-U-Z-Z-R-Z-K-K-Z-Z-X₁-K-Z-Z-X₂-Z
wherein
Z is any canonical amino acid;
U is His, Lys or Arg;
n is an integer in the range of 0 to 10 and
X₁ and X₂ are amino acids, the side chains of which are linked by a covalent linkage.

In one embodiment, the peptide of the invention comprises or consists of the sequence:

U-V-F-R-L-K-K-W-I-X₁-K-V-I-X₂-Z-F-G-Z
wherein
Z is any canonical amino acid;
U is His, Lys or Arg;
X₁ and X₂ are amino acids, the side chains of which are linked by a covalent linkage.

In one embodiment, the peptide of the invention comprises or consists of the sequence:

V-F-R-L-K-K-W-I-X₁-K-V-I-X₂-Q-F-G-E
wherein
X₁ and X₂ are amino acids, the side chains of which are linked by a covalent linkage.

In one embodiment, the peptide comprises or consists of the sequence:

U-U-V-F-R-L-K-K-W-I-X₁-K-V-I-X₂-Z-F-G-Z
wherein
Z is any canonical amino acid;
U is His, Lys or Arg; and
X₁ and X₂ are amino acids, the side chains of which are linked by a covalent linkage.

In one embodiment, the peptide comprises or consists of the sequence:

U-U-V-F-R-L-K-K-W-I-X₁-K-V-I-X₂-Q-F-G-E
wherein
U is His, Lys or Arg; and
X₁ and X₂ are amino acids, the side chains of which are linked by a covalent linkage.

In one embodiment of the invention, the peptide comprises or consists of the sequence as set forth in:
i) SEQ ID NO: 3
ii) SEQ ID NO: 4
iii) SEQ ID NO: 5
iv) SEQ ID NO: 6
v) SEQ ID NO: 7
vi) SEQ ID NO: 8;
vii) SEQ ID NO: 9;
viii) SEQ ID NO: 10;
ix) SEQ ID NO: 11
x) SEQ ID NO: 17;
xi) SEQ ID NO: 18;
xii) SEQ ID NO: 19;
xiii) SEQ ID NO: 20;
xiv) SEQ ID NO: 21;
xv) SEQ ID NO: 22;
xvi) SEQ ID NO: 23;
xvii) SEQ ID NO: 24;
xviii) SEQ ID NO: 25; or
   wherein X₁ and X₂ are amino acids, the side chains of which are linked by a covalent linkage.

In one embodiment of the invention, the peptide comprises or consists of the sequence as set forth in:
i) SEQ ID NO: 3
ii) SEQ ID NO: 4
iii) SEQ ID NO: 5
iv) SEQ ID NO: 8; or
v) SEQ ID NO: 9
   wherein X₁ and X₂ are amino acids, the side chains of which are linked by a covalent linkage.

In one embodiment, the peptide comprises or consists of the sequence as set forth in:
i) SEQ ID NO: 3
ii) SEQ ID NO: 4
iii) SEQ ID NO: 5
iv) SEQ ID NO: 8; or
v) SEQ ID NO: 9
   wherein X₁ and X₂ are (S)-2-(4′-pentenyl)-alanines, the side chains of which have been reacted with each other to form an alkenyl tether.

In one embodiment, the peptide comprises or consists of the sequence as set forth in:
i) SEQ ID NO: 3
ii) SEQ ID NO: 4
iii) SEQ ID NO: 5
iv) SEQ ID NO: 8; or
v) SEQ ID NO: 9
   wherein X₁ and X₂ are Ala, the side chains of which are covalently bound to each other by a C₈ alkenyl tether comprising one double bond.

In one embodiment, the peptide comprises or consists of the sequence as set forth in:
i) SEQ ID NO: 3
ii) SEQ ID NO: 5
iii) SEQ ID NO: 6; or
iv) SEQ ID NO: 7
   wherein X₁ and X₂ are amino acids, the side chains of which are linked by a covalent linkage.

In one embodiment, the peptide comprises or consists of the sequence as set forth in:
v) SEQ ID NO: 3
vi) SEQ ID NO: 5
vii) SEQ ID NO: 6; or
viii) SEQ ID NO: 7
   wherein X₁ and X₂ are (S)-2-(4′-pentenyl)-alanines, the side chains of which have been reacted with each other to form an alkenyl tether.

In one embodiment, the peptide comprises or consists of the sequence as set forth in:
ix) SEQ ID NO: 3
x) SEQ ID NO: 5
xi) SEQ ID NO: 6; or
xii) SEQ ID NO: 7
   wherein X₁ and X₂ are Ala, the side chains of which are covalently bound to each other by a C₈ alkenyl tether comprising one double bond.

In one embodiment, the peptide comprises or consists of the sequence as set forth in:
i) SEQ ID NO: 3
ii) SEQ ID NO: 5; or
   wherein X₁ and X₂ are amino acids, the side chains of which are linked by a covalent linkage.

In one embodiment, the peptide comprises or consists of the sequence as set forth in:
i) SEQ ID NO: 3
ii) SEQ ID NO: 5; or
   wherein X₁ and X₂ are (S)-2-(4′-pentenyl)-alanines, the side chains of which have been reacted with each other to form an alkenyl tether.

In one embodiment, the peptide comprises or consists of the sequence as set forth in:
iii) SEQ ID NO: 3
iv) SEQ ID NO: 5; or
   wherein X₁ and X₂ are Ala, the side chains of which are covalently bound to each other by a C₈ alkenyl tether comprising one double bond.

### Longer Peptides

In some embodiments of the invention, the peptide of the invention is a longer peptide that comprises at least two staples and between 24 and 40 amino acids from thrombin of SEQ ID NO: 1 and may have one of the peptide sequences described herein in this section. In addition to the peptide having the sequences described in this section, it is preferred that the peptide also:
comprises a consecutive sequence of in the range of 24 to 40 amino acids, preferably in the range of 25 to 30 amino acids from thrombin of SEQ ID NO: 1
has a total length of between 24 to 40 amino acids, preferably between 25 and 30 amino acids
comprises at least amino acids K247, K248 and K252 of SEQ ID NO: 1, preferably comprises at least amino acids R245, K247, K248 and K252 of thrombin of SEQ ID NO: 1.

In some embodiments of the invention, the peptide of the invention is a longer peptide that comprises at least two staples. In addition to the peptide having the sequences described in this section, it is preferred that the peptide also:
comprises a consecutive sequence of in the range of 24 to 25 to amino acids from GKY25 of SEQ ID NO: 12
has a total length of between 24 to 40 amino acids, preferably between 25 and 30 amino acids
comprises at least amino acids K13, K14 and K18 of GKY25 of SEQ ID NO: 12, preferably comprises at least amino acids R11, K13, K14 and K18 of GKY25 of SEQ ID NO: 12.

In a preferred embodiment, the longer peptide of the invention comprises or consists of the sequence as set forth in:
i) SEQ ID NO: 14; or
ii) SEQ ID NO: 26
   wherein X₁ and X₂ are amino acids, the side chains of which are linked by a covalent linkage; and further wherein X₃ and X₄ are amino acids, the side chains of which are linked by a covalent linkage. X₁, X₂, X₃ and X₄ in the sequences of this section may for example be as described herein above in the section “Internal covalent linkage”. Preferably, X₁ and X₂ are (S)-2-(4′-pentenyl)-alanines, the side chains of which have been reacted with each other to form an alkenyl tether, and X₃ and X₄ are Gly and Glu, respectively, which have been reacted with each other to form a lactam bridge.

### Peptide Function

As mentioned herein above, the peptides may have one or more of the following functions.

High In Vivo and/or In Vitro Stability

Preferably, the peptide has high in vivo stability. Thus, the peptide according to the invention preferably has increased stability in vivo and/or in vitro compared to a peptide with the same sequence except that the amino acids X₁ and X₂ are exchanged for other amino acids lacking the covalent linkage, when said peptides are tested under the same conditions.

In vivo stability may for example may be determined by determining anti-inflammatory activity in a mouse after administration of the peptide over time. If significant anti-inflammatory activity is maintained after e.g. 24 h, peptides have high in vivo stability.

In particular, the anti-inflammatory effect of the peptide may preferably be maintained 24 h in vivo after systemic administration of the peptide.

The in vivo stability can for example be determined as described in Example 1 herein below.

### Increased Protease Resistance

Preferably, the peptide of the invention has increased protease resistance. Thus, the peptide may have increased resistance to one or more proteases compared to a peptide with the same sequence except that the amino acids X₁ and X₂ are exchanged for other amino acids lacking the covalent linkage.

Preferably, the peptide of the invention has increased stability in the presence of a protease, such as human neutrophil elastase (HNE), Pseudomonas elastase (PE), and/or trypsin, compared to a peptide of same sequence except that the amino acids X₁ and X₂ are exchanged for other amino acids lacking the covalent linkage, when said peptides are tested under the same conditions.

Said resistance to protease may for example be determined as described in Example 1 below.

### High Anti-Inflammatory Activity

Preferably the peptide of the invention has a high anti-inflammatory activity. Even more preferably, the peptide of the invention has both anti-microbial activity and anti-inflammatory activity.

The anti-inflammatory activity may be determined in different ways, however, it may in particular be determined by inducing inflammation in a controlled way, and determining whether inflammation is reduced. The inflammation may for example be induced by contacting a reporter cell or a blood sample in vitro with LPS or by administering LPS to an animal. Inflammation may for example determined by measuring the levels of one or more proinflammatory cytokines or by determining NF-κB activity.

Thus, it is preferred that the peptide according to the invention reduces the secretion of pro-inflammatory cytokines in the presence of one or more endotoxins, such as LPS. In particular, it is preferred that the peptide decreases secretion of pro-inflammatory cytokines in vivo in blood comprising one or more endotoxins, such as LPS Said pro-inflammatory cytokines may for example be selected from the group consisting of tumour necrosis factor α (TNF-α), interleukin β (IL-1β), interleukin 6 (IL-6), interleukin 10 (IL-10), interferon (IFN-γ) and/or monocyte chemoattractant protein-1 (MCP-1). Preferably, the inflammatory cytokines are TNF-α and/or IL-1β.

It is preferred that the peptide according to the invention decreases NF-kB activation in the presence of a toll-like receptor (TLR)-agonist, such as lipopolysaccharide (LPS), lipoteichoic acid (LTA), Staphylococcus aureus peptidoglycan (SA-PGN) and/or zymosan.

It is preferred that a concentration of 10 μM of the peptide of the invention reduces secretion of TNF-α and or IL-1β after incubation in fresh blood in the presence of LPS by at least 50%, such as by at least 60%, for example by at least 70%, compared to the level in the presence of a peptide of same sequence except that the amino acids X₁ and X₂ are exchanged for other amino acids lacking the covalent linkage, when said peptides are tested under the same conditions.

In one embodiment the peptides of the invention are capable of reducing release of TNF-α in LPS stimulated blood by 50% at a concentration of less than 10 μM, such as less than 7 μM, for example in the range of 1 to 10 μM, such as at a concentration in the range of 1 to 7 μM.

The anti-inflammatory activity may be tested by one of the methods described herein below in Examples 1 and 2.

### Anti-Microbial Activity

The peptides of the invention preferably has anti-microbial activity. Whereas the peptides may not have improved anti-microbial activity compared to other thrombin derived peptides, it is preferred that they have at least some anti-microbial activity combined with increased anti-inflammatory activity.

Thus, the peptides of the invention may have bactericidal activity against Gram negative and/or Gram positive bacteria. Interestingly, the peptides of the invention have been shown effective against Gram negative bacteria. Without being bound by theory it is believed that this effect is mediated by binding of the peptides to LPS and/or other membranes structures. The peptides of the invention have also been shown effective against Gram positive bacteria. Without being bound by theory it is believed that this effect is mediated by binding of LTA and/or other membranes structures.

Thus, the peptides may have bactericidal activity mediated through the peptide being capable of killing bacteria by damaging the bacterial membrane.

In particular, it is preferred that the peptide of the invention has bactericidal effect against one or more bacteria selected from the group consisting of Staphylococcus aureus, Pseudomonas aeruginosa and Escherichia coli. Preferably, the peptide has bactericidal effect against all of the aforementioned bacteria.

Anti-microbial activity or bactericidal activity may for example be determined by radial diffusion assay or by a viable count assay. Said assays may for example be performed as described herein below in Examples 1 and 2.

### Low Hemolytic Activity in Blood

It is very preferred that the peptides of the invention have low hemolytic activity in blood. Peptides having high hemolytic activity may be toxic and are thus less suitable for systemic administration.

In general, a hemolytic activity up to 10% is acceptable. Thus, it is preferred that the hemolytic activity of the peptides of the invention is less than 10%, in particular, the hemolytic activity of the peptides of the invention in fresh, whole blood is preferably less than 10%.

More preferably, the hemolytic activity of the peptides of the invention is less than 10% at a peptide concentration with effective anti-inflammatory activity. In a preferred embodiment, the peptide of the invention has a hemolytic activity of less than 10%, at a peptide concentration which reduces anti-inflammatory activity by 50%. Said anti-inflammatory activity is preferably determined as release of TNF-α in LPS stimulated blood.

Thus, it is preferred that the hemolytic activity of the peptides of the invention is less than 10% at a peptide concentration, where said peptide is capable of reducing release of TNF-α in LPS stimulated blood by 50%.

In one embodiment, the hemolytic activity of the peptides of the invention is less than 5% at a peptide concentration, where said peptide is capable of reducing release of TNF-α in LPS stimulated blood by 50%.

Release of TNF-α in LPS stimulated blood may preferably be determined as described in Example 1 or 2 herein below.

In some embodiments, when 50 μM of the peptide is incubated in fresh, whole blood, the hemolytic activity is at the most 5%, such as at the most 4%, for example at the most 3%, such as at the most 2%.

In some embodiments, when 20 μM of the peptide is incubated in 25% fresh, whole blood, the hemolytic activity is at the most 5%, such as at the most 4%.

The hemolytic activity is preferably determined by incubating fresh blood with the peptide of the invention and determining the hemolysis and comparing to hemolysis in fresh blood in a control prepared by incubating fresh blood with a detergent, such as Tween-20. Preferably, the hemolytic activity is provided as the % hemolysis compared to the control.

The hemolytic activity may in particular be determined as described herein below in Examples 1 and 2.

### Low Toxicity

The peptides of the invention preferably also have low toxicity allowing systemic administration of the peptides.

### Structure

The peptides of the invention preferably has a substantially alpha helical secondary structure in aqueous solution. Said substantially alpha helical secondary structure is preferably maintained upon binding a target molecule, such as cluster differentiation 14 (CD14).

Alpha helical secondary structure in aqueous solution is preferably determined by circular dichroism spectroscopy.

Furthermore, the peptides preferably have a low degree of oligomerisation. The hydrodynamic radii is an indication of the oligomerisation, and thus the peptides of the invention preferably have a low hydrodynamic radii. Preferably, the peptides of the invention have hydrodynamic radii of less than 150 nm at pH 7.4 and/or less than 100 nm at pH 5. In some embodiments, it is preferred the peptides of the invention have hydrodynamic radii of less than 100 nm at pH 7.4 and/or less than 80 nm at pH 5.

Said hydrodynamic radii is preferably determined by dynamic light scattering as described herein below in Example 4.

### Anti-Coagulant Activity

The peptides of the invention may also have anti-coagulant activity. Thus, preferably the peptides of the invention have an ability to increase the time for blood to form clots. In particular, it is preferred that the peptides are able to increase the clotting time of the intrinsic pathway of coagulation. Said clotting time may preferably be determined by determining the “Activated Partial Thromboplastin Clotting Time” (aPTT). Preferably, the peptide of the invention increases the clotting time as determined by aPPT by at least 100% at a concentration of 60 μM. In some embodiments, the peptide of the invention increases the clotting time as determined by aPPT by at least 90% at a concentration of 40 μM. peptide. Said increase in the clotting time is compared to the clotting time in the absence of peptide.

Said clotting time may in particular be determined by aPTT as described herein below in Example 3.

### Reduced Haemolytic Activity on Purified RBCs

The peptides of the invention preferably have a low haemolytic activity on purified RBC at a peptide concentration with effective anti-inflammatory activity. Thus, it is preferred that the peptides of the invention have a haemolytic activity on purified RBCs of less than 75%, more preferably less than 65%, for example less than 60% at a concentration corresponding to IC₅₀ in terms of TNF-α release and/or IL-β release in LPS stimulated blood. Said haemolytic activity on purified RBCs may preferably be determined as described in Example 1 in the section Hemolysis Assay.

### Method of Treatment

The peptides of the invention are useful for treatment of various clinical conditions. Accordingly, the invention provides the peptides disclosed herein for use as a medicament.

In particular, the peptides of the invention may be for use in a method of treatment and/or prevention of inflammation in an individual in need thereof. Said method usually comprise administering a therapeutically effective amount of the peptide to said individual.

The peptides of the invention may also be for use in a method of treatment and/or prevention of infection in an individual in need thereof.

In particular, the peptides of the invention are useful for combined treatment or prevention of inflammation and infection, for example for treatment of inflammation associated with an infection in an individual in need thereof.

The peptides may be administered by any useful manner, however, the peptides are particularly useful for systemic administration, such as parenteral administration. For example, the peptides may be for subcutaneous or intravenous administration. The peptides may also be administrated by pulmonary administration, e.g. by inhalation. Other administration routes include intratecheal and intraperitoneal administration.

The individual to be treated may be any individual in need thereof, for example a human being.

The infection to be treated with the peptide of the invention may be infection with a microorganism, such as infection with a microorganism selected from the group consisting of bacteria, fungi, virus and protozoa. Thus, for example the peptides may be for use in treatment of bacterial infection, fungal infection, or a viral infection or treatment of conditions associated with such infections.

Thus, the individual may suffer from a bacterial infection. The bacterial infection may be an acute or chronic bacterial infection. Non-limiting examples of conditions to be treated with the peptides of the invention includes acute respiratory distress syndrome (ARDS), pneumonia or sepsis.

In one embodiment the peptides of the invention are for use in the treatment of an inflammatory disease. Said inflammatory disease may for example be selected from the group consisting of acute respiratory distress syndrome (ARDS), systemic inflammatory response syndrome (SIRS), severe acute respiratory syndrome (SARS), gastroenteritis, and pulmonary inflammation, e.g. pneumonitis or inflammation of the lung tissue.

The infection to be treated with the peptides of the invention may be infection may any infectious bacteria. For example, the bacteria may be Gram, negative or Gram positive bacteria. Thus, the bacteria may for example be of a genus selected from the group consisting of Staphylococcus, Enterococcus, Streptococcus, Corynebacterium, Escherichia, Klebsiella, Stenotrophomonas, Shigella, Moraxella, Acinetobacter, Haemophilus, Pseudomonas and Citrobacter.

In one embodiment, the individual to be treated has an increased level of endotoxin, such as an increased level of LPS, LTA, zymosan and/or SA-PGN. Said individual may have an increased level of endotoxin in one or more body fluids. Said body fluid may for example be selected from the group consisting of blood, serum, saliva, nasopharyngeal swab samples and bronchoalveolar lavage (BAL) samples. Said endotoxin may in particular be LPS. Said increased level of LPS may be a level of at least 50 pg/ml, such as a serum level of LPS of at least 50 pg/ml.

The individual may also suffer from a viral infection, e.g. infection with a Spike glycoprotein containing virus (also referred to as “S protein virus” herein), for example a virus of the Coronaviridae family, such as a virus selected from the group consisting of the virus is selected from the group consisting of
PorCov-HKU15,
SARS-CoV,
HCoV NL63
HKU1,
MERS-CoV
SARS-CoV 2, and
MERS-CoV.

The peptides of the invention may be administered alone or in combination with other therapeutic agents, such as antibiotic, anti-inflammatory or antiseptic agents such as anti-bacterial agents, anti-fungicides, anti-viral agents, and anti-parasitic agents.

The present invention concerns both humans and other mammal such as horses, dogs, cats, cows, pigs, camels, among others. Thus, the peptides of the invention are for use in both human therapy and veterinary applications. The objects, suitable for such a treatment may be identified by well-established hallmarks of an infection, such as fever, puls, culture of organisms, and the like. Infections that may be treated with the molecules include those caused by or due to microorganisms. Examples of microorganisms include bacteria (e.g. Gram-positive or Gram-negative), fungi, (e.g., yeast and molds), parasites (e.g., protozoans, nematodes, cestodes and trematodes), viruses, and prions and mixtures thereof. Specific organisms in these classes are well known (see for example, Davis et al., Microbiology, 3.sup.rd edition, Harper & Row, 1980).

In one embodiment of the invention, the peptides disclosed herein are for use in treatment or prevention of a disease, condition or indication, which for example may be any of the diseases, conditions or indications described below.

Acute systemic inflammatory disease, with or without an infective component, such as systemic inflammatory response syndrome (SIRS), ARDS, sepsis, severe sepsis, urosepsis, and septic shock. Other invasive infective and inflammatory disease, including meningitis, arthritis, toxic shock syndrome, diverticulitis, appendicitis, pancreatitis, cholecystitis, colitis, pneumonia, urinary tract infections and peritonitis.

Chronic inflammatory and or infective diseases, including cystic fibrosis, COPD and other pulmonary diseases, gastrointestinal disease including chronic stomach ulcerations.

Inflammatory and coagulative disorders including thrombosis or disseminated intravascular coagulation (DIC). Furthermore, vasculitis related inflammatory disease, as well as allergy, including allergic rhinitis and asthma.

Inflammation in relation to, but not limited to, stroke, extracorporeal circulation procedures such as ECMO, cardiopulmonary bypass, or ex vivo lung perfusion process.

Excessive contact activation and/or coagulation in relation to, but not limited to, stroke, extracorporeal circulation procedures such as ECMO, cardiopulmonary bypass, or ex vivo lung perfusion process.

Excessive inflammation in combination with antimicrobial treatment.

For example, the peptides of the invention may be for use in the treatment or prevention of an acute inflammation, sepsis, acute respiratory distress syndrome (ARDS), systemic inflammatory response syndrome (SIRS), chronic obstructive pulmonary disease (COPD), cystic fibrosis, asthma, allergic and other types of rhinitis, vasculitis, thrombosis and/or disseminated intravascular coagulation (DIC).

In one embodiment, the peptides of the invention exhibits both anti-inflammatory and anti-coagulant activity and may be used in the concomitant treatment or prevention of inflammation and coagulation. Such peptides may be particularly suited to the treatment and prevention of conditions where the combined inhibition of both inflammatory and coagulant processes is desirable, such as ARDS, sepsis, chronic obstructive pulmonary disorder (COPD), thrombosis, DIC and acute respiratory distress syndrome (ARDS). Furthermore, other diseases associated with excessive inflammation and coagulation changes may benefit from treatment by the peptides, such as cystic fibrosis, asthma, allergic and other types of rhinitis, and vasculitis.

### Preparation of Peptide

Methods for the production of peptides are well known in the art.

Peptides may be produced by recombinant methods well known in the art (see e.g. Sambrook & Russell, 2000, Molecular Cloning, A Laboratory Manual, Third Edition, Cold Spring Harbor, New York).

Alternatively, peptides may be chemically synthesized, e.g. by linking multiple amino acids via amide bonds. Typically, peptides are chemically synthesized by the condensation reaction of the carboxyl group of one amino acid to the amino group of another. Protecting group strategies may be used to prevent undesirable side reactions with the various amino acid side chains.

Well known liquid-phase or solid phase peptide synthesis techniques are known to the skilled person (such as standard f-Boc or Fmoc solid-phase peptide synthesis).

The covalently linkage of the side chains of two, non-neighbouring internal amino acids may be introduced by any method known to the skilled person, such as for example by any of the methods described by Li et al., 2020.

When X₁ and X₂ are alkenylated amino acids, such as olefin terminated amino acids, then the hydrocarbon staple may be introduced using ring-closing metathesis (RCM), for example by ruthenium catalysed ring-closing metathesis or by RCM using Grubbs' first-generation catalyst in 1,2-dichloroethane. RCM may be performed in solution or on solid supports, and multiple ways are described in Li et al., 2020 as well as in Example 1.

Similarly, when X₃ and X₄ are alkenylated amino acids, such as olefin terminated amino acids, then the hydrocarbon staple may be introduced using ring-closing metathesis (RCM), for example by ruthenium catalysed ring-closing metathesis or by RCM using Grubbs' first-generation catalyst in 1,2-dichloroethane. RCM may be performed in solution or on solid supports, and multiple ways are described in Li et al., 2020 as well as in Example 1.

When X₃ is an N-terminal amino acid and/or an amino acid comprising an amine group, and X₄ is an amino acid comprising a carboxylic acid side chain, then the covalent linkage may be an amide bond between said amine and said carboxylic acid. In other words, the covalent linkage may be an amide bond, i.e. a lactam bridge, between said amine and carboxylic acid. Such cyclization may be performed on solid phase.

Peptides according to the invention can also be ordered from companies specialised in producing custom made peptides, for example from AmbioPharm Inc. (US).

### EXAMPLES

### Example 1

### Materials and Methods

### Peptides

The peptides GKY25 (GKYGFYTHVFRLKKWIQKVIDQFGE) (SEQ ID NO: 12), HVF18 (HVFRLKKWIQKVIDQFGE) (SEQ ID NO: 2), and their respective stapled versions denoted as sGKY25 (GKYGFYTHVFRLKKWIXKVIXQFGE) (SEQ ID NO: 13), 2sGKY25 (cyclo[GKYGE]YTHVFRLKKWIXKVIXQFGE) (SEQ ID NO: 14) and sHVF18 (HVFRLKKWIXKVIXQFGE) (SEQ ID NO: 3) were synthesised by AmbioPharm, Inc. (USA). Briefly, standard 9-fluorenylmethyloxycarbonyl (Fmoc) solid-phase peptide synthesis (SPSS) was used. To obtain hydrocarbon stapled peptides, olefin-bearing (S)-2-(4′pentenyl)-alanine was inserted at specified locations in the respective peptide sequences (denoted by X) in SEQ ID NOs: 3 and 13. Olefin metathesis reaction was performed on solid support using Grubbs' first-generation catalyst in 1,2-dichloroethane.

The product peptides were cleaved from the resin and further purified by RP-HPLC.

Peptides were provided as acetate salts, and the purity was confirmed with MALDI-TOF MS (>95%). Cyclization of 2sGKY25 was performed using solid phase conditions, by forming an amide bond, i.e. a lactam bridge between the N-terminal amine of Gly1 and the carboxylic acid side chain of Glu5 of said peptide.

### Biological Materials

Venous blood was collected from healthy donors, after written informed consent was obtained. After collection, whole blood or its fraction, such as plasma and serum, were used immediately or were stored at −80° C. The use of blood was approved by the Ethics Committee at Lund University, Lund, Sweden (Permit Number DNR2015/801).

### Circular Dichroism Spectroscopy

The secondary structure of GKY25, HVF18 and their respective stapled versions, with and without Lipopolysaccharides from Escherichia coli O111:B4 (LPS, Sigma-Aldrich, USA) was assessed by Circular dichroism (CD). The peptides were diluted to 10 μM in 10 mM Tris at pH 7.4 and incubated with 100 μg mL⁻¹ LPS for 30 min at 37° C. A Jasco J-810 spectropolarimeter (Jasco, USA) equipped with a Jasco CDF-426S Peltier set to 25° C. was used to perform the measurements. The spectra were recorded between 190-260 nm (scan speed: 20 nm min⁻¹) as an average of 5 measurement in a 0.2 cm quartz cuvette (Hellma, GmbH & Co, Germany). The baseline (10 mM Tris pH 7.4±100 μg mL⁻¹ LPS) was subtracted from each spectra and the final signal was converted to mean residue ellipticity, θ (mdeg cm² dmol⁻¹) as reported by Morrissette et al.

In another set of experiments linear and stapled version of HVF18 were mixed with 25 or 50% TFE or in water with 100 μg mL⁻¹ LPS, then spectra were acquired as reported above.

### RP-HPLC

HVF18 and its stapled version (2.5 μg) was injected in reverse-phase C18-column (Phenomenex Kinetex 50×2.1 mm 2.6 μM, 100 Å pore size, California, USA) by using Agilent 1260 Infinity System following the protocol reported by Petruk et al. Biomolecules (2020). Briefly, the column was equilibrated using 95% of buffer A containing 0.25% of TFA in MilliQ and 5% of Buffer B containing 0.25% of TFA in Acetonitrile. The peptide was pre-mixed with Buffer A (1:3) 5 min before being loaded on the column.

Peptides were digested with different proteases for different lengths of time as described below, and then injected in reverse-phase C18-column. The analysis was performed as above. Samples from two different digestions were analysed.

### Proteolysis of the Peptides In Vitro

Peptides were resuspended in endotoxin free water at concentration of 1 mM. Then, 20 μg of GKY25 and sGKY25 or 14.7 μg of HVF18 and sHVF18 were incubated with 0.2 μg of human neutrophil elastase (HNE, Calbiochem®, Merk KGaA, Darmstadt, Germany), P. aeruginosa elastase (PE, Calbiochem®, Merk KGaA, Darmstadt, Germany), Glutamyl-C endopeptidase (EC 3.4.2.11.9) from S. aureus V8 (BioCol GmbH, Michendorf, Germany) or trypsin (Try, Promega, Madison, WI, USA) for different lengths of time (0-18 h) in a final volume of 20 μL. At the end of the incubation, the digestion was evaluated by Tricine SDS-PAGE and mass spectrometry (for GKY25, sGKY25, HVF18 and sHVF18), and by RP-HPLC (for HVF18 and sHVF18). All digestions were performed in three independent experiments.

### SDS-PAGE

Two μg of the peptide from each condition were loaded on 10-20% Novex Tricine pre-cast gel from Invitrogen (USA). The run was performed at 100 V for 100 min. The gel was stained by using Coomassie Brilliant blue (Invitrogen, USA). The image has been acquired with a Gel Doc Imager (Bio-Rad Laboratories, USA). Samples from three independent digestions were analysed.

### NMR Spectroscopy

The NMR experiment was performed on a 700 MHz Bruker Avance Ill HD spectrometer (Swedish NMR Centre, Gothenburg, Sweden), equipped with QCI cryo-probe and pulse field gradients. Sample was prepared by dissolving 1.6 mM sHVF18 in 50% 2,2,2-Trifluoroethanol (TFE), supplemented with 10% D₂O, 200 μM DSS, 0.02 vol % NaN₃ at pH 4.5. ¹H spectra were acquired from the sample at 298 K. Based on the ¹H spectra and TOCSY spectra (80 ms mixing time), a series of TOCSY (40 and 80 ms mixing time), NOESY (100 and 150 ms mixing time), ROESY (100 and 150 ms mixing time), DQF-COSY, ¹³C-edited HSQC, ¹⁵N-SOFAST-HMQC, ¹³C HSQCTOCSY and ¹³C-HMBC spectra were acquired. Spectra were processed using nmrPipe with squared cosine apodization and zero-filling in both dimensions. Spectra were analyzed and assigned using CCPNMR v2.4. Spin systems were identified using a combination of NOESY and TOCSY spectra, where NOESY cross-peaks were used to assign inter-residue connections.

### NF-κB Activation Assay

Anti-inflammatory activity of four peptides was tested on THP1-XBlue-CD14 reporter cells (InvivoGen, San Diego, USA). Briefly, 180,000 cells well⁻¹ were seeded in 96 well plates in phenol red RPMI media, supplemented with 10% (v v⁻¹) heat-inactivated FBS and 1% (v v⁻¹) Antibiotic-Antimycotic solution (AA). Then 100 ng mL⁻¹ LPS (Sigma, USA) with and w/o peptides at different concentrations (1-20 μM) were added. The NF-κB activation was determined after 20 h of incubation according to the manufacturer's instructions (InvivoGen, San Diego, USA), i.e. by mixing 20 μL of supernatant with 180 μL of SEAP detection reagent (Quanti-Blue™, Invivo-Gen), followed by absorbance measurement at 600 nm. Data shown are mean values±SEM obtained from at least four independent experiments all made in triplicate. In another set of experiment, the cells were stimulated with 100 ng ml⁻¹ of E. coli LPS, 1 μg ml⁻¹ S. aureus LTA, 1 μg ml⁻¹ E. coli PGN, 1 μg ml⁻¹ S. aureus PGN, 10 μg ml⁻¹ S. cerevisiae zymosan in the presence or the absence of 10 μM of linear and stapled HVF18. The experiment was performed as above. The activation of NF-κB was evaluated as described previously.

### Hemolysis Assay

Fresh venous blood from healthy donors was collected in in lepirudine tubes (50 μg mL-1). Then 50 μL of blood were transferred to round-bottom 96-wells plate containing 150 μL of peptides previously diluted in RPMI-1640—GlutaMAX-I without phenol red (Gibco). 1:4 diluted blood was used as control. 50 μL of blood mixed with 150 μl 5% Tween-20 were used as positive control. After 1 h incubation at 37° C. and 5% CO₂, the plate was centrifuged at 800 g, 150 μL of each sample were transferred to a flat-bottom 96-wells plate and the absorbance at 450 nm was measured. The percentage of hemolysis was calculated following the formula reported below:

$$\frac{Abs 450 nm ( Sample - control )}{Abs 450 nm ( Posivite control - control )} \times 100$$

To evaluate the hemolytic effect of the peptides on erythrocytes the blood was collected as reported above and centrifuged at 250 g for 10 min. Then plasma was discarded, and red blood cells were washed with 150 mM NaCl in 10 mM Tris pH 7.4 for three times. Next, the pellet was dilute 100 times with saline Tris buffer. 100 μL of this solution were added to round-bottom 96-wells plate containing 100 μL of peptides previously diluted in saline Tris buffer. After 1 h incubation at 37° C. and 5% CO₂, the plate was centrifuged and the absorbance at 450 nm was measured, then the percentage of erythrocyte lysis was determined using formula reported above.

### CD14 Expression and Purification

Human His-tag CD14 (hCD14-his) was produced in insect cells and purified as reported previously²³.

### Microscale Thermophoresis

A NanoTemper Monolith NT.115 apparatus (Nano Temper Technologies, Germany) was used to perform Microscale thermophoresis (MST). A Monolith NT Protein labelling kit RED—NHS (Nano Temper Technologies, Germany) was used to label 687 μL (20 μM) of recombinant hCD14 according to the manufacturer's protocol. hCD14 (5 μL of 21 nM) was incubated with increasing concentrations of GKY25, HVF18 and their stapled versions (0.03-1000 μM) in 10 mM Tris at pH 7.4 with or w/o 150 mM NaCl in a ration 1:1. Then, the sample was loaded into standard glass capillaries (Monolith NT Capillaries, Nano Temper Technologies), and the MST analysis was performed (settings for the light-emitting diode and infrared laser were 80%). Results shown are mean values±SD of six measurements.

### Whole Blood Assay

Fresh venous blood was collected in the presence of lepirudin (50 μg mL⁻¹) from healthy donors. The blood was diluted 1:4 in RPMI-1640-GlutaMAX-1 (Gibco) and 1 mL of this solution was transferred to 24-well plates and stimulated with 100 ng mL⁻¹ of LPS immediately after adding increasing concentrations of GKY25, HVF18 and their stapled versions. After 24 h incubation at 37° C. in 5% CO₂, the plate was centrifuged for 5 min at 1000 g and then the supernatants were collected and stored at −80° C. before analysis. The experiment was performed at least 4 times by using blood from different donors each time. To evaluate the healing properties of the peptides, the blood was stimulated with 100 ng mL⁻¹ LPS and after 30 min incubation at 37° C. was treated with increasing doses of four peptides. In the last set of experiments, the preventive anti-inflammatory activity of the peptides was evaluated by exposing the blood to increasing concentrations of GKY25, HVF18 and their stapled versions for 30 min. Then the blood was stimulated with 100 ng mL⁻¹ of LPS.

### Cytokine Assay

Plasma obtained from blood experiment was used to evaluate cytokine release. Human inflammation DuoSet® ELISA Kit (R&D Systems) specific for TNF-α and IL-1β was used according to the manufacturer's instructions. Absorbance was measured at a wavelength of 450 nm. Data shown are mean values±SEM obtained from at least four independent experiments all performed in duplicate.

The level of TNF-α, IFN-γ, MCP-1, IL-10 and IL-6 in murine plasma were assessed using the Mouse Inflammation Kit, (Becton Dickinson AB) according to the manufacturer's instructions.

### Mouse Inflammation Model

The immunomodulatory effects of HVF18 and sHVF18 were studied in BALB/c tg(NF-κB-RE-Luc)-Xen reporter mice (Taconic, 10-12 weeks old). The peptide (50 μg mouse⁻¹) was injected subcutaneously simultaneously with E. coli LPS (25 μg mouse⁻¹) in a final volume of 200 μL. The dorsums of the mice (8 mice per treatment group) were carefully shaved and cleaned. After injection, animals were immediately transferred to individually ventilated cages and imaged 3 h later. We used an In Vivo Imaging System (IVIS Spectrum, PerkinElmer Life Sciences) for determination of NF-κB activation. Bioluminescence from the mice was detected and quantified using Living Image 4.0 Software (PerkinElmer Life Sciences). Fifteen minutes before the IVIS imaging, mice were intraperitoneally given 100 μL of D-luciferin (150 mg/kg body weight).

### LPS Model In Vivo

E. coli 0111:B4 LPS was resuspended in 10 mM Tris pH 7.4. Then sublethal dose (6 mg per kg of body weight) were injected intraperitoneally (i.p.) into male C57BL/6 mice (11-12 weeks, 22+/−5 g). Thirty minutes after, 10, 20, 50, 100 or 500 μg sHVF18 (in 10 mM NaOAc pH 5) per mouse were injected i.p. into the mice. After 8 and 20 h post LPS challenge, mice were deeply anesthetized by isoflurane and the blood was collected by cardiac puncture and stored at −80° C. until further analysis.

### Bacterial Cell Culture

One colony of E. coli ATCC 25922, S. aureus ATCC 29213, P. aeruginosa O1 as well as of 8 clinical isolates of S. aureus and 7 of P. aeruginosa, was inoculated in 5 mL of Todd-Hewitt (TH) medium overnight at 37° C. with shaking. The day after, the bacteria cell culture was diluted 1:50 in fresh TH media and left to grow to mid-logarithmic phase. Then, the bacteria were centrifuged at 3500 g for 10 min, washed and subsequently resuspended in 10 mM Tris pH 7.4 at final concentration of 2×10⁹ colony forming units (CFU) mL⁻¹.

### Radial Diffusion Assay (RDA)

Bacteria were grown and prepared as described above. The microorganisms (4×10⁶ CFU) were then added to 15 mL of the underlay agarose gel, consisting of 0.03% (w/v) TSB, 1% (w/v) low electroendosmosis type agarose (Sigma-Aldrich) and 0.02% (v/v) Tween 20 (Sigma-Aldrich). The underlay gel was poured into a 144-mm diameter petri dish. After agarose solidification, 4-mm diameter wells were punched and 6 μL peptide solution of required concentration were added to each well. Plates were incubated at 37° C. for 3 h to allow peptide diffusion. The underlay gel was then covered with 15 mL of molten overlay gel (6% TSB and 1% low electroendosmosis type agarose in distilled H₂O). The activities of the peptides are presented as clear zone-to-well diameter (excluding the 4 mm well). All the experiments were performed at least 4 times.

### Viable Count Assay (VCA)

Bacteria were grown and prepared as described above. Next, the bacterial suspension was diluted 1:1000 in 10 mM Tris pH 7.4 at concentration 2×10⁶ CFU mL⁻¹. Bacteria (50 μL) were incubated with different concentrations of GKY25, sGKY25, HVF18 and sHVF18 (1-20 μM) in 10 mM Tris pH 7.4 with or without 150 mM NaCl or 25% of human citrate-plasma, for 2 h at 37° C. At the end of incubation, serial dilutions of the samples were plated on TH agar plates, incubated overnight at 37° C., and the CFU were calculated. Bacteria treated with respective buffer were used as a control. All the experiments were performed at least 4 times. Data shown are mean values±SEM.

### Live/Dead Assay

The permeability of the bacteria membrane was evaluated by LIVE/DEAD BacLight™ Bacterial Viability kit (Invitrogen, Molecular Probes, Carlsbad, CA, USA) as previously described²⁴. Briefly, the bacterial suspension was prepared as for VCA. S. aureus and P. aeruginosa O1 suspension (50 μL) was then treated by 1 μM or 5 μM HVF18 and its stapled version in 10 mM Tris at pH 7.4, respectively. The buffer was used as negative control. After 2 h, the samples were mixed with 1 μL of the dye mixture for each mL of the bacterial suspension, as reported on the manufacturer's protocol, and incubated in the dark at room temperature for 15 min. At the end of incubation, 5 μL of the stained bacterial suspension were trapped between a slide and an 18-mm square coverslip. We examined 10 view fields (1×1 mm) of the mounted samples from three independent sample preparations using a Zeiss AxioScope A.1 fluorescence microscope (objectives: Zeiss EC Plan-Neofluar 100/1.3 oil; camera: Zeiss AxioCam MRm; acquisition software: Zeiss Zen 2.6 (blue edition).

### Transmission Electron Microscopy

The effect of the peptides on S. aureus and P. aeruginosa O1 membrane was further evaluated by Transmission electron microscopy (TEM) (Jeol Jem 1230; Jeol, Japan) in combination with negative staining. In particular, 5 μL bacterial suspension from VCA were adsorbed onto carbon coated grids (Copper mesh, 400) for 60 s and stained with 7 μL of 2% uranyl acetate for 30 s. The grids were rendered hydrophilic via glow discharge at low air pressure. Analysis was done on 10 view fields (magnification '4200) of the mounted samples on the grid (pitch 62 μm) from three independent experiments.

### MIC and MBC Assay

The minimal inhibitory concentration (MIC) was determined according to the protocol reported by Wiegand et al., Bacteria were grown and diluted as described above. Next, the bacteria were further diluted 1:1000 in 2×BBL™ Mueller Hinton II (MH), cation adjusted broth (Becton, Dickinson and Company, Sparks, USA). Bacteria (50 μL) were added to 96-well round bottom polystyrene plates (Corning INC, Kennebunk, USA) containing 50 μL 2×MH broth (control), or 2×MH broth with peptide (HVF18 or sHVF18) at concentration ranging from 2.5-320 μM. The plates were then incubated at 37° C. for 24 h. MIC was validated as the lowest concentration at which no visible bacterial growth was observed. Following analysis of the MIC, the same plate was used to determine minimal bactericidal concentration (MBC). For this purpose, the sample for each condition was resuspended using a pipette tip, and a droplet of 10 μL was plated on a THA plates and incubated at 37° C. overnight. MBC was fixed at the concentration at which no bacterial colony formation was observed.

### Results

The Design of Dual-Action Peptides with Improved Proteolytic Stability.

Thrombin C-terminal peptide GKY25 has a proven dual-action that targets both bacterial infection and the associated TLR-driven inflammation. One approach to increase peptide stability is peptide hydrocarbon stapling which is a modification that stabilizes peptide secondary structure with a side-chain covalent hydrocarbon bridge. Stapling has been applied to other peptides, although the effect of stapling is difficult to predict. A hydrocarbon staple moiety was introduced into the sequence of GKY25 (GKYGFYTHVFRLKKWIQKVIDQFGE) by substituting certain amino acids with (S)-2-(4′pentenyl)-alanine. The position of stapling was decided based on the information for GKY25 summarized in Table 1.

TABLE 1

| GKY25 | Amino acid marked in bold and |
|---|---|
| (SEQ ID NO: 12) | underlined: |
| GKYGFYTHVFRLK | evolutionarily conserved |
| KWIQKVIDQFGE | |
| GKYGFYTHVFRLK | positively charged, important |
| KWIQKVIDQFGE | for LPS binding |
| GKYGFYTHVFRLK | hydrophobic, might be important |
| KWIQKVIDQFGE | for interaction with CD14 |
| GKYGFYTHVFRLK | interacts with K87 from CD14 |
| KWIQKVIDQFGE | |
| GKYGFYTHVFRLK | Preferred for stapling |
| KWIQKVIDQFGE | |

Furthermore, the centrally located lysine residues (K13, K14 and K18—numbers in this section refers to the GKY25 sequence (SEQ ID NO: 12)) may be important for GKY25's antimicrobial activity. Furthermore, protonation of H8 at pH 5.5 increases the antibacterial activity of GKY25 against Gram-negative Escherichia coli by membrane disruption. GKY25 binds to LPS and the LPS-binding hydrophobic pocket of CD14 and the residues responsible for LPS and CD14 interaction have been indicated in Table 1.

Studies demonstrate that K14 cross-links to K87 in CD14 and in silico docking studies show that the C-terminal residues of Q17, K18, D21, Q22, and E25 are exposed to the solvent. NMR studies have determined the LPS bound conformation (PDB code 5z5x) in which the C-terminal α-helix starts at 116. Interactions with LPS are mediated with hydrophobic residues and the positively charged residues H8, R11, K13 and K14. If disregarding the amino acids of GKY25, which may have one of the aforementioned activities, amino acids Q17, D21, Q22 and E25 remained. Since Q22 and E25 are close to the end, stapling in the position of Q17 and D21 was introduced.

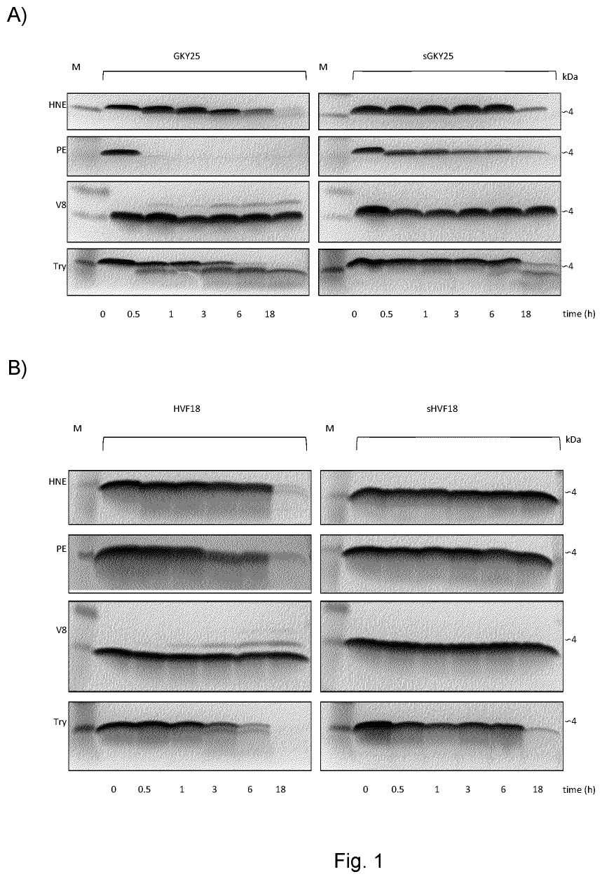

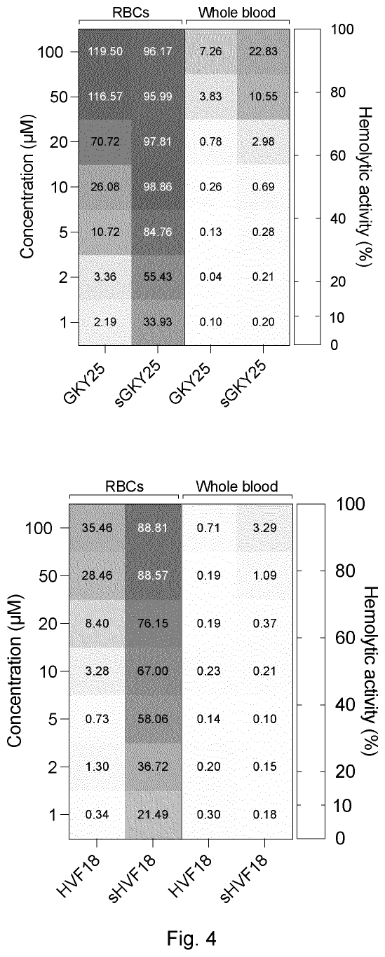

The helicity of stapled GKY25 (herein denoted as sGKY25) in comparison to its native versions were analysed using circular dichroism (CD). The spectrum of GKY25 in the presence of LPS was used as a positive control. sGKY25 showed an α-helical structure and the content of the helicity was comparable to GKY25 when it is bound to LPS. In case of sGKY25 with LPS, the α-helical content remained unchanged. To confirm that stapling was increasing the proteolytic stability of the peptide we exposed it to various proteases for different length of time and then analyzed it using SDS-PAGE. As shown in FIG. 1a, stapling was enhancing the stability of GKY25 in the presence of HNE up to 6 h. The stability was also increased against trypsin. Indeed, it was still possible to detect intact sGKY25 after 6 h of digestion. Interestingly, none of the peptides was susceptible to V8 digestion, even though its cleavage site is present in the sequence. To understand which regions were released from the digested sGKY25 in comparison to its native form, we employed LC-MS/MS. As expected, linear peptide showed wide variety of fragments released already after 30 min of digestion. On the other hand, the stapling was conferring a partial protection to sGKY25. Few fragments corresponding to C-terminal part of sGKY25 were found after digestion with PE and none with HNE, at time points analyzed. This is not surprising, since it is known that N-terminal part of GKY25 is more flexible and more prone to proteolysis. The stapling of GKY25 resulted in a more hemolytic construct specially towards red blood cells (RBCs) (FIG. 4a), however sGKY25 also had significant hemolytic activity in whole blood (FIG. 4a). Even at a concentration of 50 μM sGKY25 had a hemolytic activity in whole blood above 10%, which is typically the upper limit of acceptable hemolytic activity.

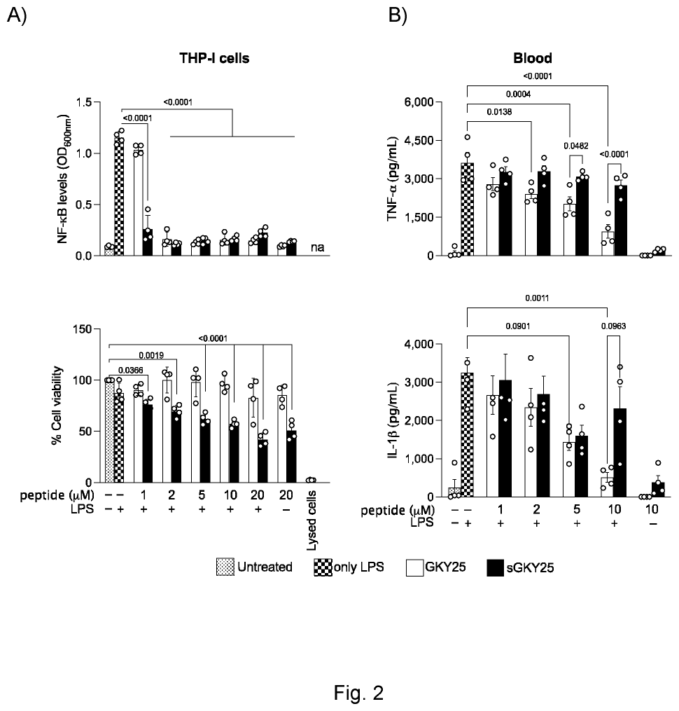

When the anti-inflammatory activity was tested on THP-I cells stimulated with LPS, sGKY25 showed significant improvement with respect to linear peptide, but also a high toxicity at higher concentrations (FIG. 2a). In a more physiological condition, i.e. in blood, the stapled sGKY25 peptide completely failed in blocking inflammation induced by LPS (FIG. 2b).

The helicity of stapled HVF18 (sHVF18) was confirmed by CD and in a hydrophobic environment such as that characteristic of RP-HPLC. sHVF18 showed longer retention time (9.42 min) if compared with linear HVF18 (8.03 min), since the functional binding surface was increased with the locking. Subsequently we tested the resistance to proteases cleavage by SDS-PAGE (FIG. 1b), HPLC and LC-MS/MS. Stapling was enhancing the stability of HVF18 up to 18 h with HNE and PE. The stability was also increased against trypsin. Indeed, it was still possible to detect intact sHVF18 even after 18 h. As for linear and stapled GKY25, HVF18 variants were not susceptible to V8 digestion. LC-MS/MS results confirmed that stapling of HVF18 was conferring the peptide complete protection to proteolysis. The helix stability of sHVF18 was further tested exposing the peptide to increasing temperatures and analyzing the secondary structure by CD. It was found that even after exposure to 80° C., sHVF18 still presented an α-helical spectrum, i.e. with two characteristic minimum at 208 and 222 nm. Notably, sHVF18 showed lower hemolytic activity if compared with sGKY25 (compare FIG. 4b with FIG. 4a). In particular, sHVF18 showed significantly lower hemolytic activity in whole blood compared to sGKY25.

The insertion of stapling in a correct place was confirmed with nuclear magnetic resonance (NMR). sHVF18 was dissolved in 50% TFE, that is known to increase the secondary structure and then TOCSY, NOESY, ROESY, ¹³C-HSQC and ¹⁵N-SOFAST-HMQC spectra were collected. Secondary structure estimations were done using the DANGLE dihedral angle and chemical shift index (CSI) module in the CCPNMR suite, which estimates that sHVF18 contains an α-helix consisting of residues 7 to 14.

A detailed nuclear magnetic resonance (NMR) study of sHVF18 was performed. The peptide was dissolved in 50% TFE. TOCSY, NOESY, ROESY, ¹³C-HSQC and ¹⁵N-SOFAST-HMQC spectra were collected on the sHVF18 in 50% TFE. TOCSY, NOESY and ROESY spectra showed well dispersed peaks, where amino acid type can be easily identified in the TOCSY spectra. NOESY and ROESY spectra show many HN-Hα and HN-HN cross peaks, allowing an easy sequential assignment of the peptide. The ¹³C HSQC spectrum shows well-dispersed peaks. 16 cross peaks corresponding to amide backbone atoms could be detected in the ¹⁵N SOFAST-HMQC spectra as well as side chain cross peaks for 8Trp and 15Gln. The ¹⁵N HMQC and ¹³C HSQC spectra indicate that sHVF18 samples have a well-defined conformational state under these conditions. The presence of multiple HN-HN (I,i+2) and HN-Hα (I, i+2/3) indicates the presence of a well-defined secondary structure. Assignments were done and 97% of the available ¹H resonances could be identified. Secondary structure estimations were done using the DANGLE dihedral angle estimations and chemical shift index (CSI) module in the CCPNMR suite, which estimates that sHVF18 contains an α-helix consisting of residues 7 to 14, which is also consistent with the NOE pattern. The stapled linker is easily seen in the NOESY spectra due to the aromaticity of the staple and based on TOCSY spectra the presence of the staple can be confirmed. The structure ensemble forms an L-shaped structure with two α-helices, consisting of residues Val2-Leu5 and Lys11-Gly17. The N-terminal and C-terminal α-helices are structurally well-defined, however there is a relatively high amount of variability in the orientation of the two α-helices with respect to one another. When the structure of sHVF18 in TFE is compared to HVF18 in the presence of LPS, it is possible to observe a similar L-shaped tertiary structure with a backbone RMSD of 2.2 Å. The main differences are seen for the N-terminal part of HVF18, where the α-helix seen in sHVF18 is not observed. This can, however, be due to the TFE inducing sHVF18 into a more helical structure than LPS inducing HVF18, as showed by CD analysis. In sHVF18, the N-terminal part of sHVF18 seems to be more ordered than in HVF18. The C-terminal α-helix in HVF18 ranges over residues Ile9-Gly17, which is two amino acids longer than sHVF18. From the sHVF18 structure ensemble, it seems like the staple breaks the α-helix. A more detailed look at the NOEs acquired for sHVF18 and comparing these to the distances in the HVF18 ensemble structure, NOE distances for the residues 5Leu, 7Lys, 8Trp, 9Ile and 11Lys are inconsistent with the HVF18 structure ensemble, showing structural differences between sHVF18 and HVF18.

### Effects of Stapling on Anti-Inflammatory Activity of the Peptide.

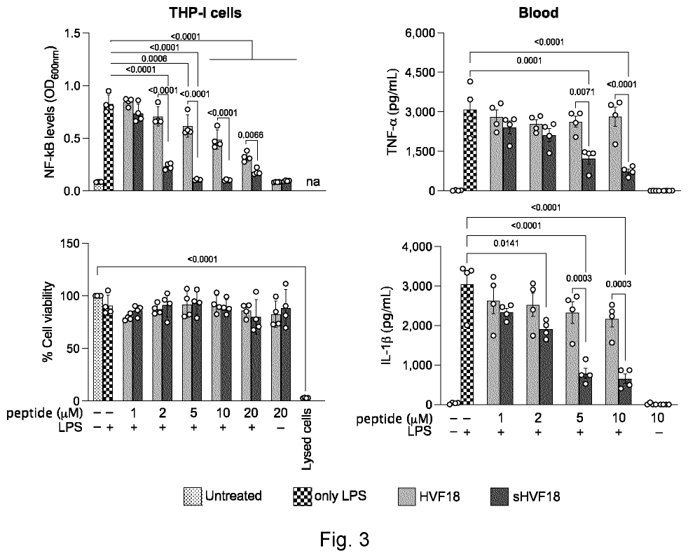

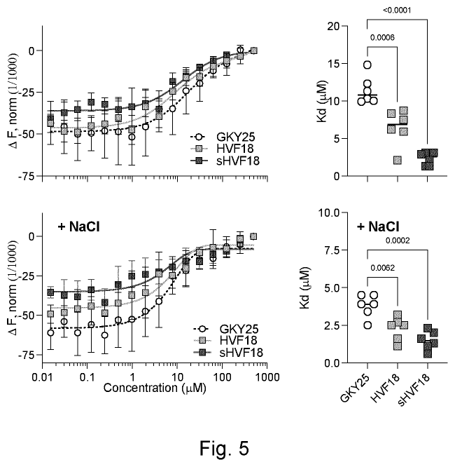

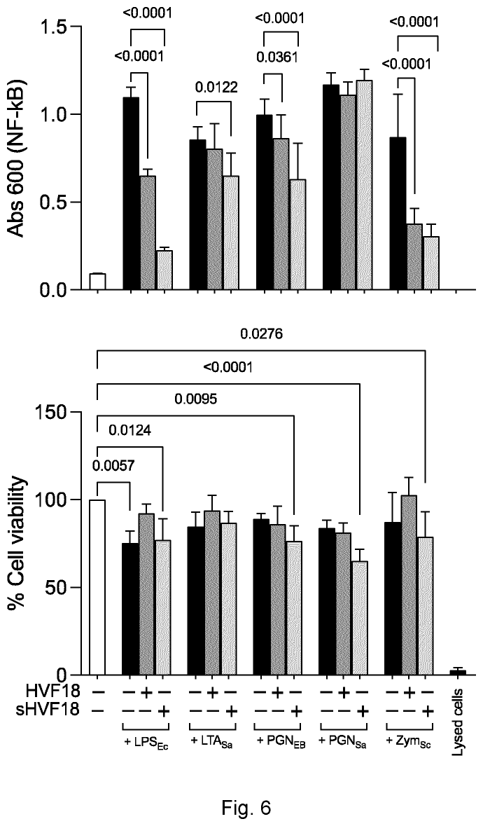

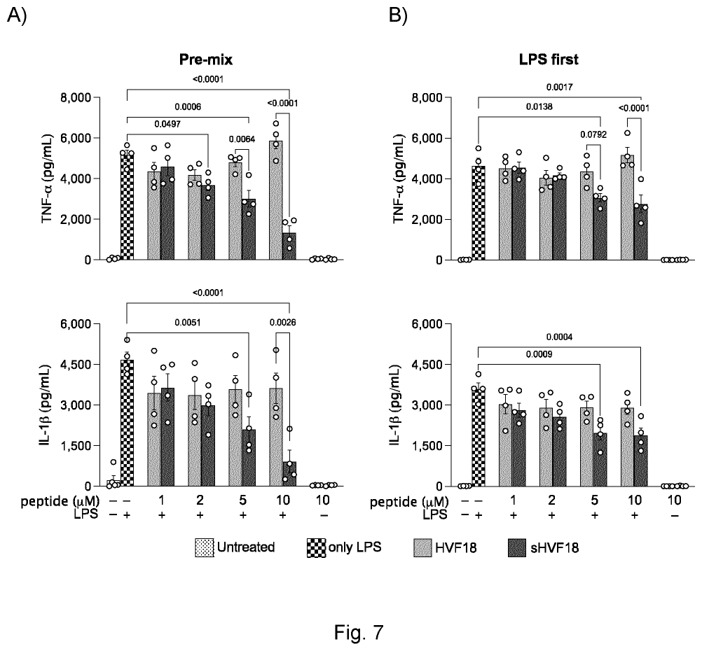

Microscale thermophoresis (MST) was used to determine K_{d}. The K_{d} of sHVF18 to CD14 was markedly decreased, even in the presence of salt (FIG. 5). THP1-XBlue-CD14 reporter cells were stimulated with LPS in the presence of increasing concentration of linear and stapled HVF18 and the NF-κB activation was evaluated. FIG. 3 clearly shows that the anti-inflammatory activity of sHVF18 was significantly improved. In addition, this was true also for other TLR-agonists, such as LTA and PGN form S. aureus, PGN from E. coli and Zymosan from S. cerevisiae (FIG. 6a). In THP-1 cells a stronger inhibition of LPS-induced NF-κB/AP-1 activation by sHVF18 was observed compared to its linear form (FIG. 3). 25% fresh venous human blood was incubated with stapled HVF18 or linear HVF18 and simultaneously stimulated with LPS for 24 h. We found that sHVF18 efficiently and in dose-dependent manner reduced TNF-α and IL-1β secretion, particularly when the peptide was added before stimulation with LPS or together (FIG. 3 and FIG. 7a). The inhibition of sHVF18 was lower, but still significant when the blood was first stimulated with LPS for 30 min and then treated with increasing doses of the peptide (FIG. 7b).

### Effects of Stapled Peptide on Endotoxin Responses in an Experimental Mouse Model.

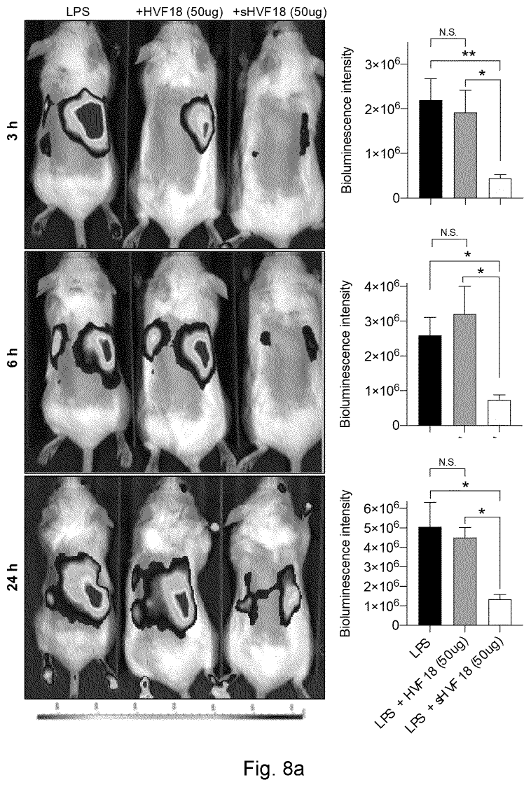

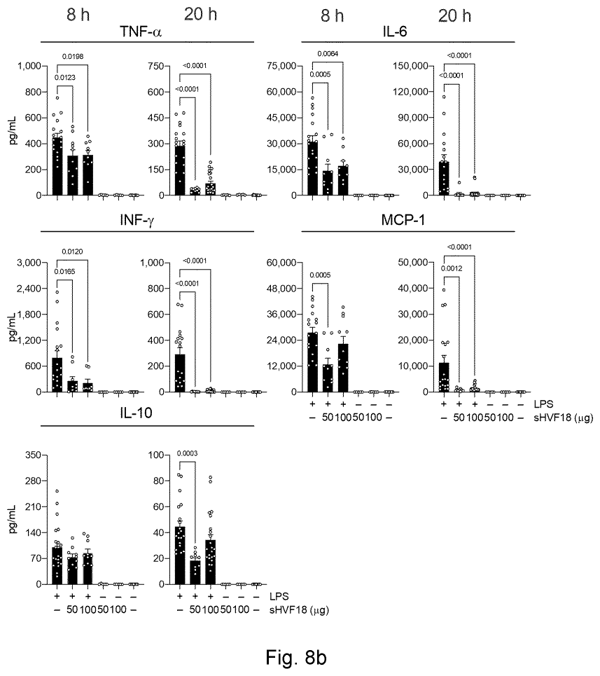

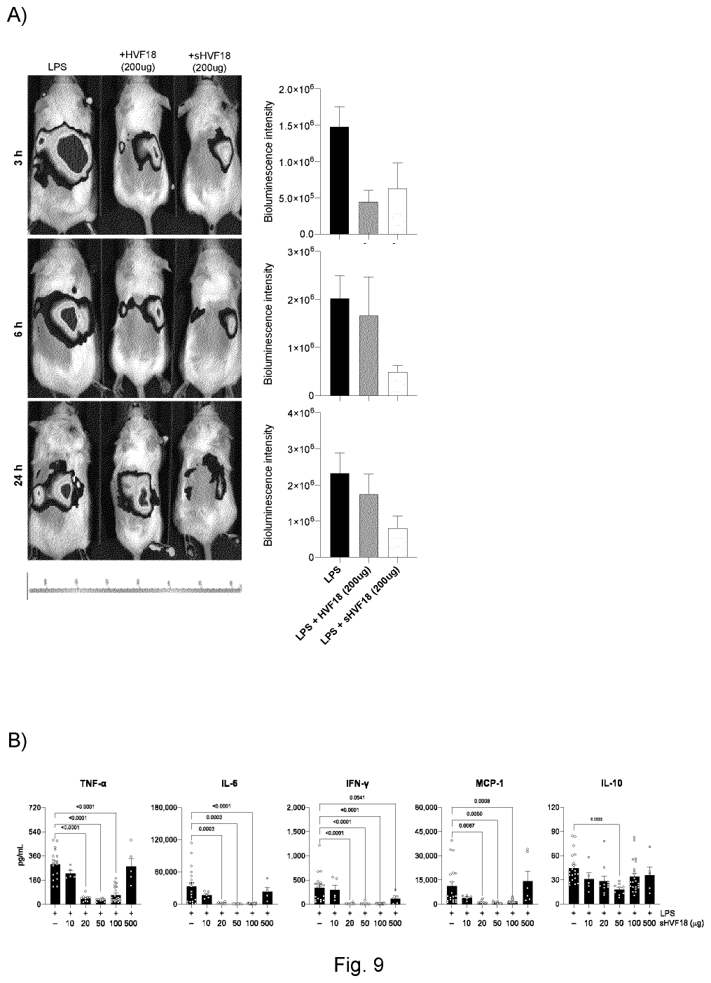

In a first set of experiments, it was explored whether sHVF18 could suppress LPS-induced local inflammation in NF-κB reporter mice. The same amount of stapled or linear peptide was injected subcutaneously, with simultaneous addition of LPS, then NF-κB activation was measured over time (FIGS. 8a and 9). In agreement with the in vitro data, sHVF18 demonstrated strong anti-inflammatory activity already at 50 μg, whereas its linear counterpart was not effective (FIG. 8a and FIG. 9). Indeed, a higher concentration of HVF18 was required (200 μg). Moreover, the activity of linear peptide was less stable over time, with a reduced inhibition of the pro-inflammatory effect induced by LPS. In the second set of experiments, the activity of sHVF18 was evaluated in a mouse model of endotoxin-induced shock. C57BL/6 mice were injected i.p. with a sublethal dose of LPS and after 30 min treated with increasing doses of sHVF18. After 20 h mice were sacrificed, and cytokine levels were analyzed in blood samples (FIG. 9b). It was observed that at 20, 50 and 100 μg of sHVF18 there was a significant reduction in pro-inflammatory cytokines, such as TNF-α, IL-6, IFN-γ and MCP-1 (FIG. 9b). Interestingly, sHVF18 at 500 μg completely failed to reduce the cytokine levels. Next, using a shorter time point of 8 h, the effect of 50 and 100 μg of sHVF18 was investigated (FIG. 8b). A lower, but still significant reduction of cytokines levels was observed for both concentrations of sHVF18.

### Effects of Stapling on Antimicrobial Activity of the Peptide.

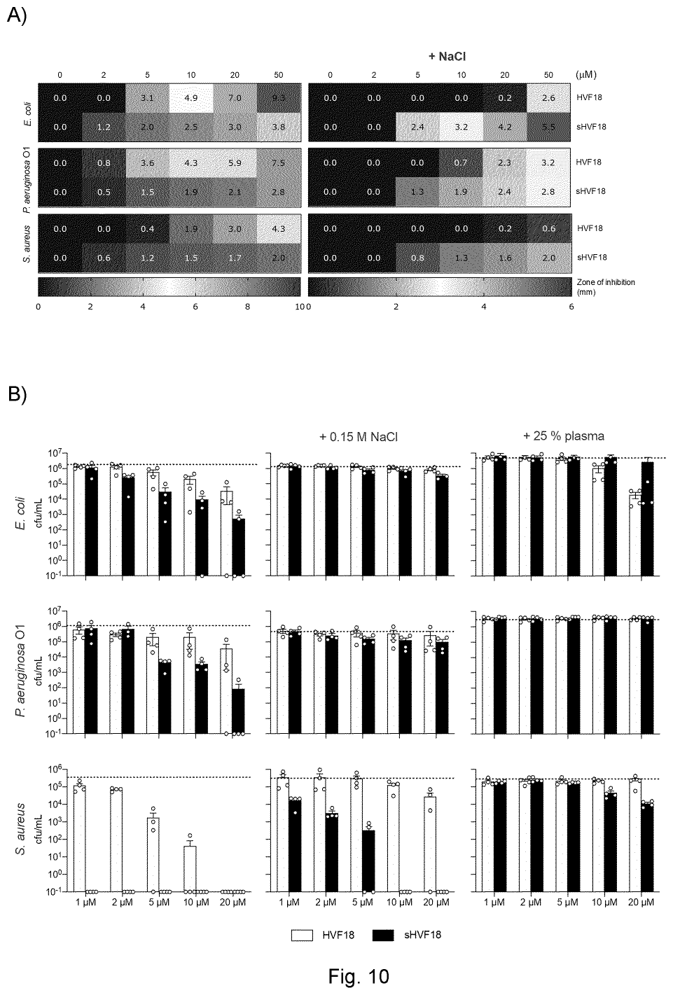

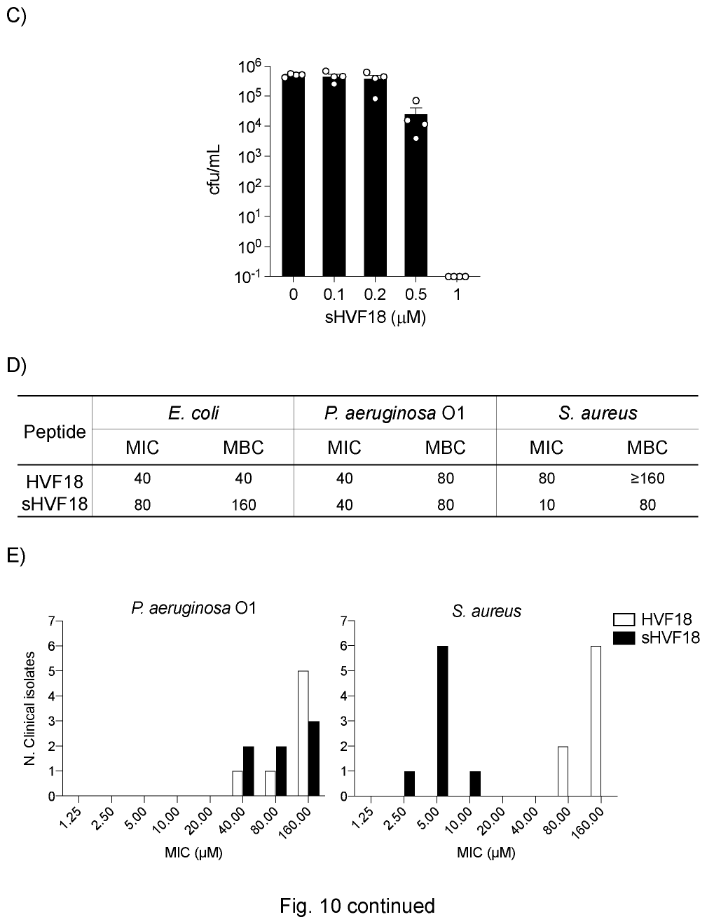

By using radial diffusion assay (RDA), it was noted that sHVF18 was active in the same manner independently of the conditions, i.e., w/o or with NaCl, whereas the linear peptide showed better bacterial killing in experiments w/o salt. Indeed, a decrease in activity was observed in the presence of NaCl (FIG. 10a). Testing the antimicrobial activity in solution, it was found that sHVF18 was more bactericidal than HVF18 on all strains evaluated (FIG. 10b, left panels). Moreover, it showed marked affinity for S. aureus, killing it at concentration lower than 1 μM (FIG. 10c). Since, one goal of this study was to use the sHVF18 as systemic drug, the activity of the peptide was evaluated also in more complex situation, such as the presence of salt or human plasma (FIG. 10b, middle and right panels). The antimicrobial activity of both peptides decreased with increased complexity of the media, but nevertheless sHVF18 showed stronger killing effect than its linear form (FIG. 10b, middle and right panels).

The antimicrobial activity of sHVF18 was further confirmed in a standard minimum inhibitory concentration (MIC) assay (FIG. 10 d, e). As in the VCA, sHVF18 showed a stronger activity towards Gram-positive bacteria.

Taken together, these results demonstrate that sHVF18 retains its antibacterial activity against Gram-negative bacteria, when compared with its linear form. Moreover, it becomes more active and thus, more selective for Gram-positive bacteria. Finally, the killing is mediated through bacterial membrane permeabilization and disruption.

### Example 2

### Materials and Methods

### Peptides

The peptides GKY25 (GKYGFYTHVFRLKKWIQKVIDQFGE)(SEQ ID NO: 12), sGKY25 (GKYGFYTHVFRLKKWIXKVIXQFGE) (SEQ ID NO: 13), 2sGKY25 (ciclo[GKYGE] YTHVFRLKKWIXKVIXQFGE)(SEQ ID NO: 14), sHVF18 (HVFRLKKWIXKVIXQFGE) (SEQ ID NO: 3), sKVF18 (KVFRLKKWIXKVIXQFGE) (SEQ ID NO: 4), sKKVF18 (SEQ ID NO: 5), sKKKVF18 (SEQ ID NO: 6), sKKKKVF18 (SEQ ID NO: 7), sRVF18 (RVFRLKKWIXKVIXQFGE) (SEQ ID NO: 8), sRRVF18 (SEQ ID NO: 9), sRRRVF18 (SEQ ID NO: 10) and sRRRRVF18 (SEQ ID NO: 11) were synthesized and purified by AmbioPharm, Inc. (USA) as reported for sHVF18. Peptides were provided as acetate salts, and the purity was confirmed with MALDI-TOF MS (>95%).

### Circular Dichroism Spectroscopy

The secondary structure of all stapled peptides was assessed by Circular dichroism (CD). The peptides were diluted to 10 μM in 10 mM Tris at pH 7.4 and spectra were measured by a Jasco J-810 spectropolarimeter (Jasco, USA) equipped with a Jasco CDF-426S Peltier set to 25° C., as reported for sHVF18. Data shown are mean values obtained from three independent experiments.

### NF-κB Activation Assay

Anti-inflammatory activity of all stapled peptides was tested on THP1-XBlue-CD14 reporter cells (InvivoGen, San Diego, USA). The experiment was performed as reported for sHVF18, with the exception that final concentration of each peptide was between 1-10 μM. Data shown are mean values±SEM obtained from four independent experiments all made in triplicate.

### Hemolysis Assay

The hemolytic effect of stapled peptides on erythrocytes or whole blood was tested as reported for sHVF18. Data shown are mean values±SEM obtained from at least four independent experiments all made in triplicate. For each experiment blood from a different donor was used.

### Whole Blood Assay

The anti-inflammatory effect of different stapled peptides in blood was performed as reported for sHVF18. For each experiment blood from a different donor was used.

### Cytokine Assay

Plasma obtained from blood experiments was used to evaluate cytokine release. Human inflammation DuoSet® ELISA Kit (R&D Systems) specific for TNF-α and IL-1β was used according to the manufacturer's instructions. Absorbance was measured at a wavelength of 450 nm. Data shown are mean values±SEM obtained from four independent experiments. For 2sGKY25, the data shown are mean values±SEM obtained from two independent experiments.

The level of TNF-α, IFN-γ, MCP-1, IL-10 and IL-6 in murine plasma were assessed using the Mouse Inflammation Kit, (Becton Dickinson AB) according to the manufacturer's instructions.

### LPS Model In Vivo

E. coli 0111:B4 LPS was resuspended in 10 mM Tris at pH 7.4. Then a sublethal dose (6 mg per kg of body weight) was injected intraperitoneally (i.p.) into male C57BL/6 mice (11-12 weeks, 22+/−5 g). Thirty minutes after, 10 μg of sHVF18, sKVF18, sKKVF18, sRVF18 or sRRVF18 (in 10 mM NaOAc at pH 5) per mouse were injected i.p. into the mice. For untreated mice, 100 μL of 10 mM Tris at pH 7.4 were injected before and 100 μL of 10 mM NaOAc at pH 5 after 30 min. After 20 h post LPS challenge, mice were deeply anesthetized by isoflurane and the blood was collected by cardiac puncture and stored at −80° C. until further analysis.

### Antibacterial Activity

Antibacterial activity of different stapled peptides on E. coli ATCC 25922, S. aureus ATCC 29213 and P. aeruginosa O1, by radial diffusion assay (RDA) or viable count assay (VCA) was performed as for sHVF18, with the exception that the highest tested concentration of each peptide was 20 and 10 μM for RDA and VCA, respectively.

### Results

### Analysis of the Structure and Hemolytic Activity of Stapled Peptides

As shown in Example 1 stapling of HVF18 increased its stability to proteolysis and greatly improved its anti-inflammatory activity (in vitro and in vivo), retaining its antimicrobial activity, but turning it mostly against Gram-positive bacteria, while at the same time having a low hemolytic activity. Different variants of sHVF18 were made, where the N-terminal His residue was exchanged with 1 to 4 Lys or Arg residues. In addition, another variant of stapled GKY25 (2sGKY25) was made, which had 2 stapled regions, one variant as in sGKY25 in the C-terminal region, but with an extra staple in the N-terminal region. Since sGKY25 failed to block LPS-induced inflammation in complex environment as human blood as described in Example 1 (see FIG. 2b), the purpose with the double-staple was to evaluate if 2sGKY25 was able to show any anti-inflammatory activity.

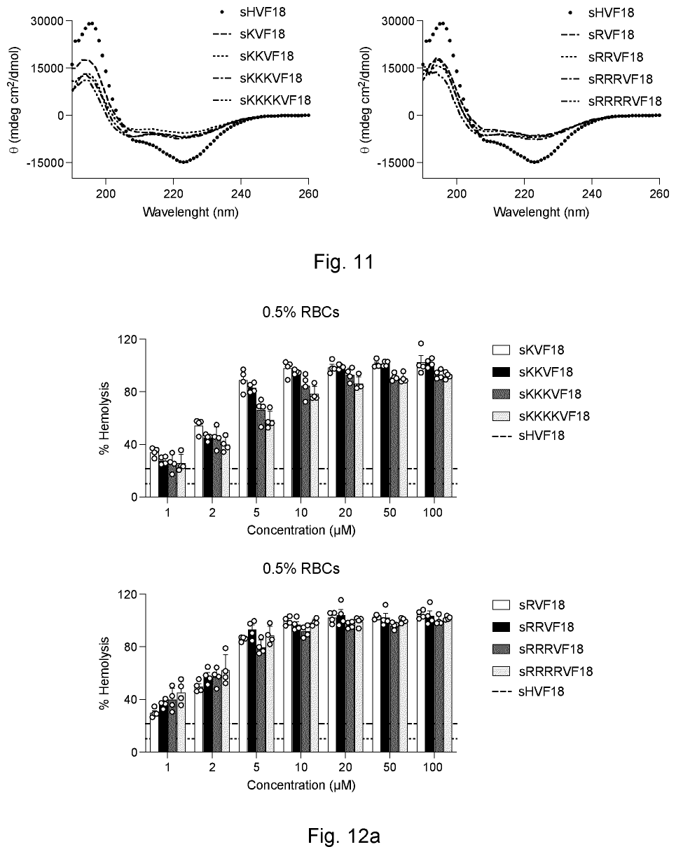

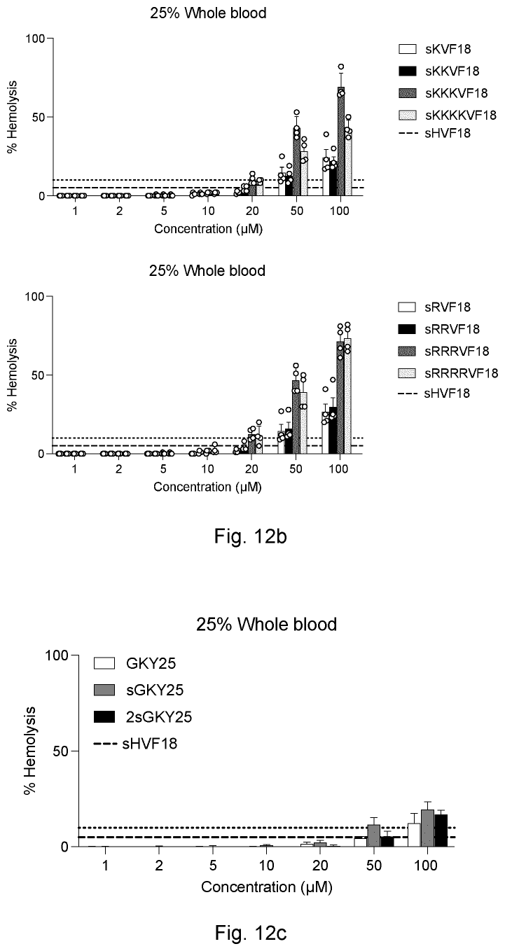

The secondary structure of sHVF18 and its variants was evaluated by circular dichroism (CD). In FIG. 11 it is possible to observe that K and R variants of sHVF18 have even a more defined α-helical structure, with two minimum one at 208 nm and one at 222 nm. The hemolytic activity of these peptides were compared on red blood cells (RBCs) and whole blood (FIGS. 12a and b, respectively). It was found that all peptides were hemolytic on RBCs. The difference in amount of K and R residues was not making any difference in this regard (FIG. 12a). When the same analysis was performed in whole blood, it was observed that the hemolytic activity of K and R variants was significantly increased when the number of positive residues was 3 or 4 as compared to 1 or 2 (FIG. 12b). When analyzing the hemolytic activity of 2sGKY25 in whole blood, it was noted that the hemolytic activity was slightly lower than for sGKY25, but definitely higher than for sHVF18 (FIG. 12c—see dashed line). The dotted line in FIG. 12 indicates 10% hemolytic activity. In general it is preferred that peptides have a hemolytic activity below 10%.

### Evaluation of Anti-Inflammatory Activity of Stapled Peptides In Vitro and In Vivo

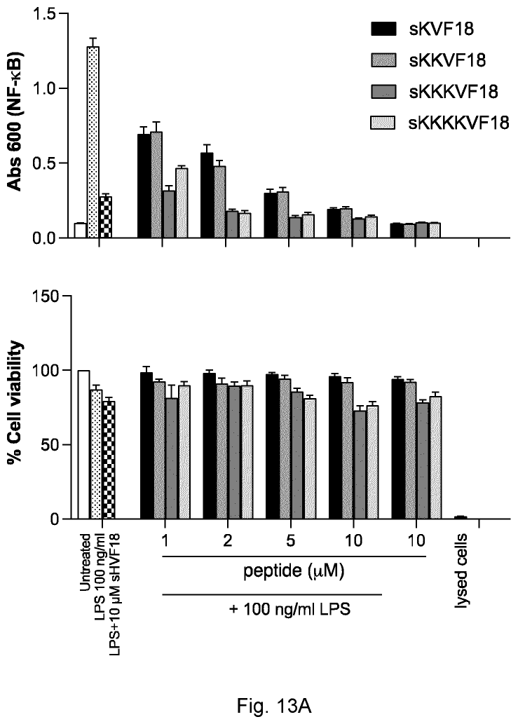

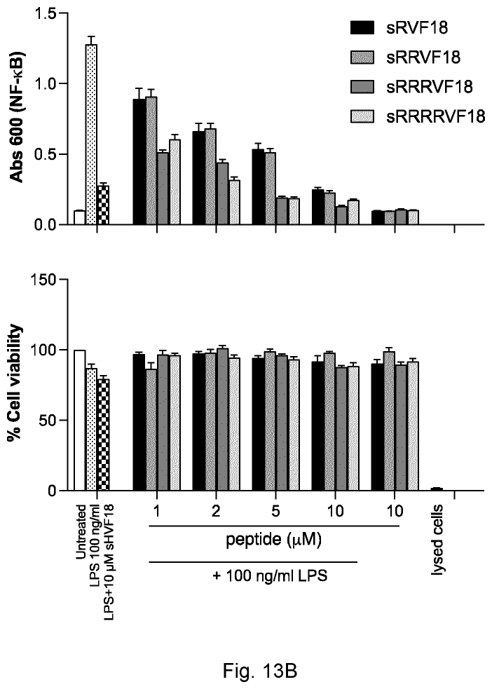

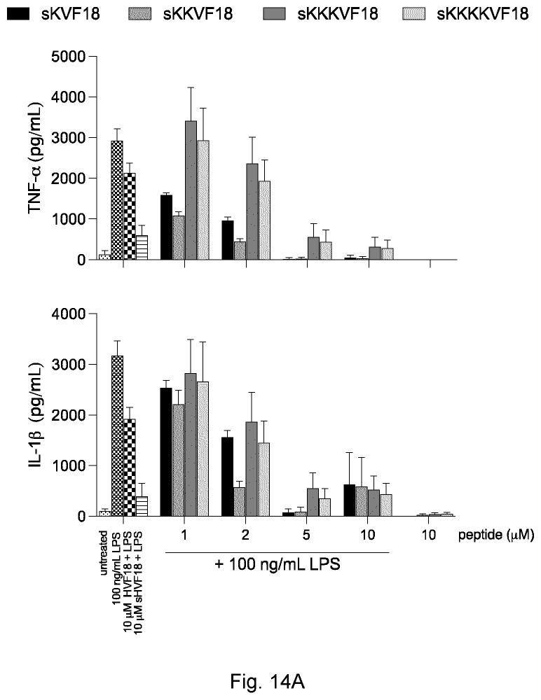

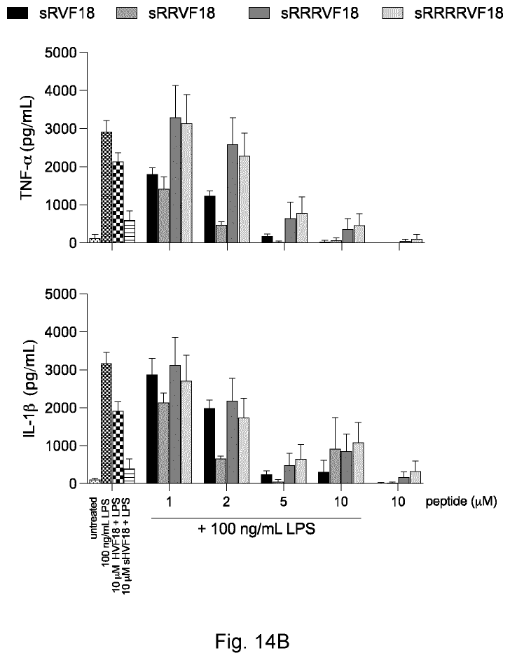

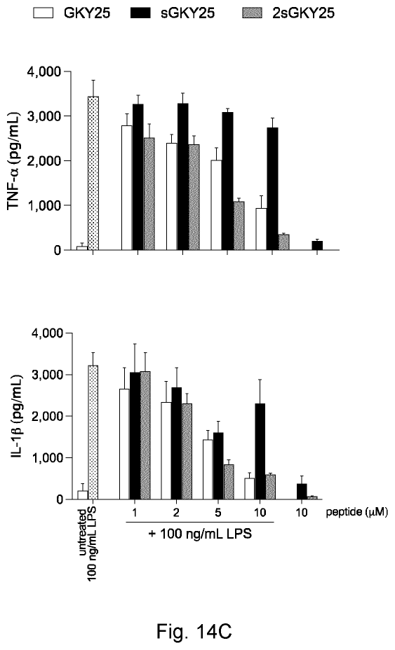

The ability of stapled peptides to counteract the LPS-induced inflammation was first evaluated in THP-1-XBlue-CD14 reporter cells. The data are reported in FIG. 13a for variants of HVF18 with K and in FIG. 13b for variants with R. All peptides showed improved activity than the original sHVF18 when tested at 10 μM as the final concentration. The variants with 3 and 4 K or R showed even better activity already at 2 and 5 μM, respectively, with respect to 10 μM sHVF18. Then we evaluated the anti-inflammatory activity in a more physiological environment, i.e. 25% of whole blood. The blood was stimulated with LPS in the presence or the absence of different peptides, and then the release of TNF-α and IL-1β in the plasma was quantified by ELISA (FIGS. 14a and b). Surprisingly, here the activity of the peptides with 3 and 4 K or R was lower than for the peptides with 1 or 2 positively charged residues. Particularly the peptides with 2 K or R showed improved effects. In particular, it was found that these peptides significantly reduced TNF-α release, even at very low concentrations of 1-2 μM, and at 5-10 μM peptide, TNF-α release was almost completely abolished. It was also found that these peptides significantly reduced IL-1β release at the 2-5 μM. We also tested the activity of double stapled GKY25 with respect to its linear and single stapled version in blood stimulated with LPS (FIG. 14c). It was found that closing GKY25 also on N-terminal region was conferring better suppression of LPS-induced TNF-α and IL-1β release than GKY25, but higher doses were needed when compared with variants of sHVF18.

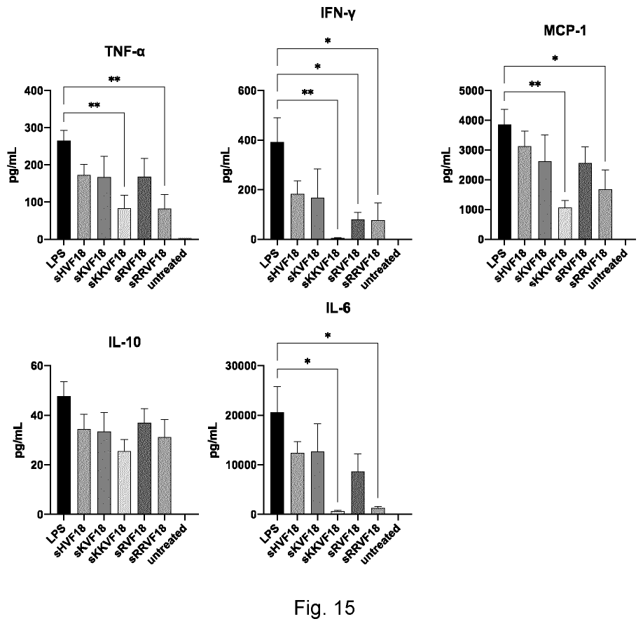

The activity of 10 μg sHVF18 with 1 and 2 K or R variants was compared in mice stimulated with LPS. We found that in mice treated with peptides, all peptides were able to reduce the levels of different cytokines. The 2 K and R variants showed strongest effects on cytokine reduction (FIG. 15).

### Evaluation of Antibacterial Activity of Stapled Peptides In Vitro

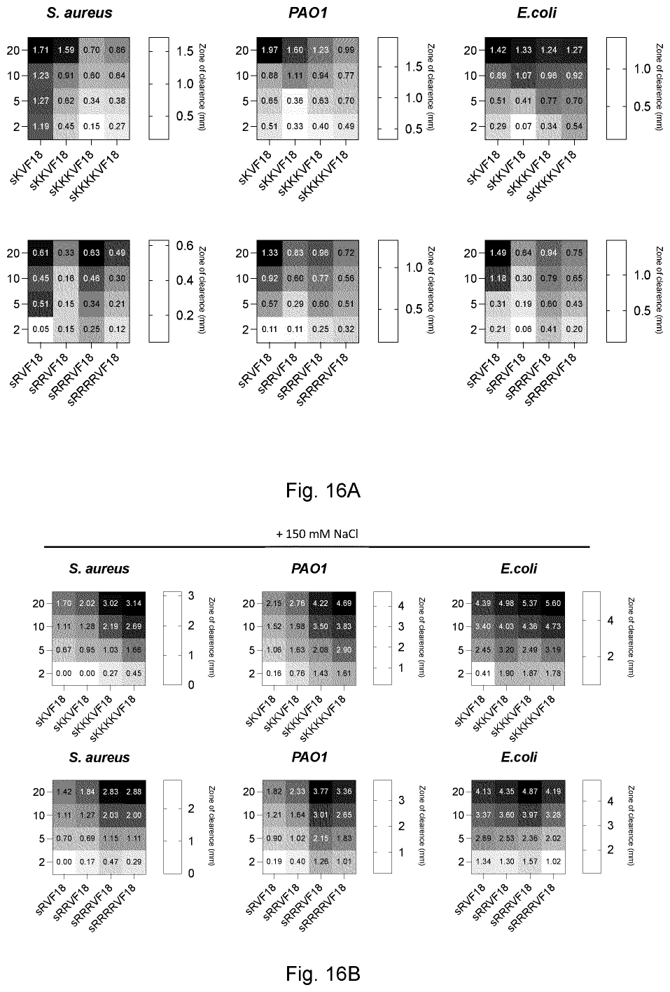

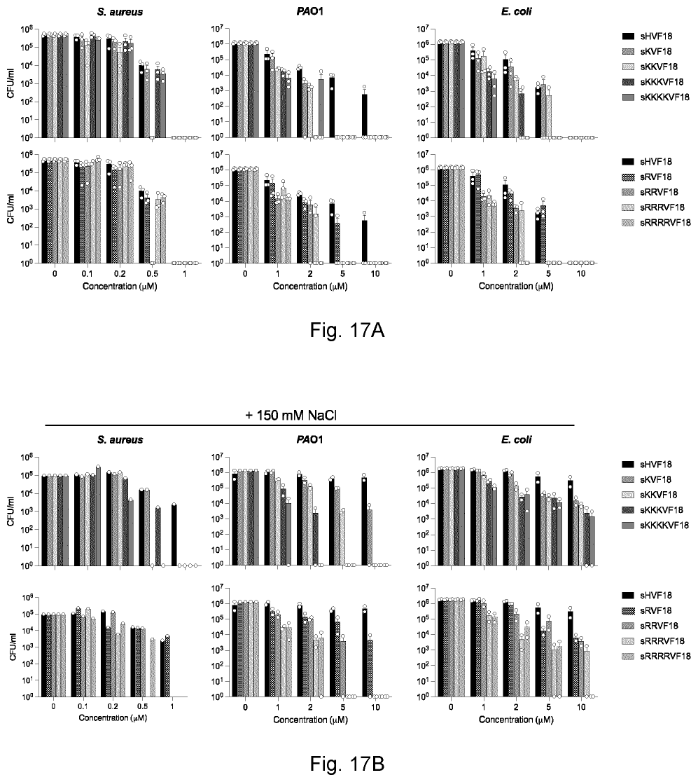

The activity of antimicrobial peptides is strongly linked to their specific sequence characteristics. The effect of K and R additions in the stapled sHVF18 variants on their activity against E. coli, P. aeruginosa O1 and S. aureus was therefore determined. The zones of clearance was measured in the absence of NaCl (FIG. 16a) and in the presence of 150 mM NaCl (FIG. 16b). It was found that in general the peptides with lower positive net charge were more active in the media without salt, and vice versa. Moreover, the ability to clear bacteria on the plate without NaCl of sHVF18 K and R variants in general was better at lower concentrations if compared to the original sHVF18. On the plates with NaCl, which is more comparable to an in vivo situation, particularly the variants with increased number of K showed a significantly improved antimicrobial activity. The bactericidal activity of the peptides in solution, again in the absence (FIG. 17a) and in the presence (FIG. 17b) of NaCl was investigated. Here it was seen that the sHVF18 with K and R variants were more active on all bacterial strains when compared to original sHVF18, in particular in the presence of salt. In addition, the activity was higher with increasing net charge of the peptides.

### Conclusions

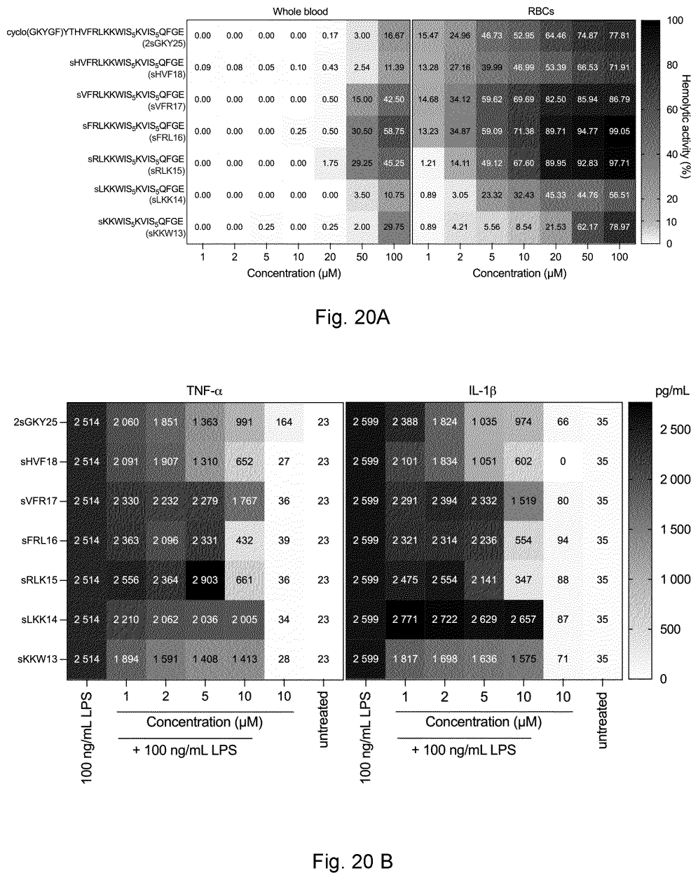

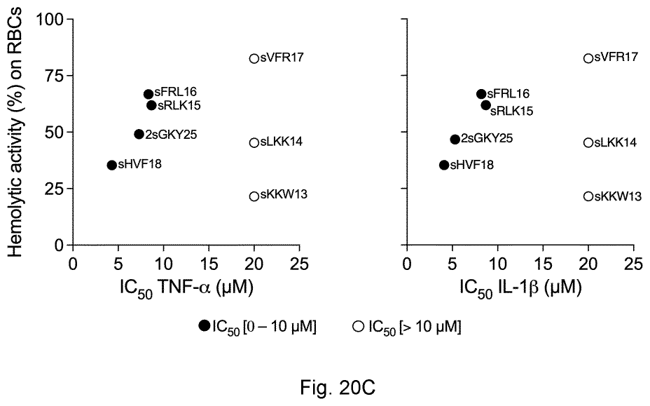

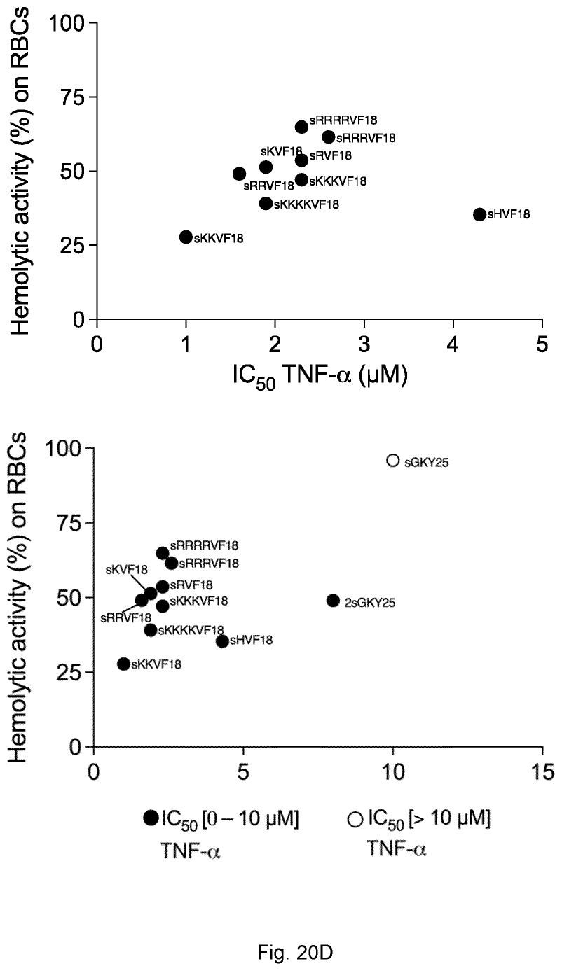

Overall results for different linear and stapled peptides investigated in Examples 1 and 2 are summarized in the Table 2 and in FIG. 20d. It is demonstrated that the anti-inflammatory activity of the stapled peptides is significantly increased in a complex environment such as human blood. In particular, the variant of sHVF18 with 2 R or K showed superior results. On the other hand, when replacing His with 3 and 4 R or K, the anti-inflammatory effect was lower than for the variants with 1 and 2 R or K, possibly due to the higher toxicity of these peptides, as demonstrated by the hemolysis results. These results indicate that there is an optimal amount of positively charged residues that can be inserted in the peptide structure, which in the present case corresponds to two residues of K or R. Both these variants showed a higher therapeutic index, i.e. a larger difference between the therapeutic concentration and the dose causing toxicity. Regarding the retention of antimicrobial activity, it was observed that replacement of His with one or more Lys or Arg residues enhances the bactericidal effect of these variants if compared with the original sHVF18, particularly in the presence of salt.

TABLE 2
The comparative hemolytic, anti-inflammatory and antimicrobial activities of linear and stapled peptides.

| | | Hemolytic activity | | | | | | | |
|---|---|---|---|---|---|---|---|---|---|
| | Anti-inflammatory activity | | lysed RBCs | Anti-bacterial activity (reuction in Log) | | | | | |
| | Peptide concentration that reduces by 50% the | lysed | in whole | S. aureus | | P. aeruginosa | | E. coli | |
| | release of TNF-α in LPS stimulated blood | RBCs | blood | RDA | VCA | RDA | VCA | RDA | VCA |
| | peptide in (μM) | % | % | (mm) | (log10) | (mm) | (log10) | (mm) | (log10) |
| GKY25 | 3.7 ± 2.1 | 7.5 ± 1.1 | <1 | 0.2 | >6 | 2.5 | 2 | 2.4 | 1 |
| sGKY25 | >10 | 96.0 ± 5.8* | 10.6 ± 5.5* | 0.2 | >6 | 1.4 | 2 | 2.9 | 1 |
| 2sGKY25 | 8 ± 2.3 | 49.1 ± 13.6 | <1 | nd | nd | nd | nd | nd | nd |
| HVF18 | >10 | 28.5 ± 3.0* | <1* | 0 | 0 | 0.7 | 0 | 0 | 0 |
| sHVF18 | 4.3 ± 1.8 | 35.4 ± 5.9 | <1 | 0.8 | 1 | 1.3 | 0 | 2.4 | 0 |
| sKVF18 | 1.9 ± 0.6 | 51.4 ± 4.9 | <1 | 0 | >6 | 0.4 | 0 | 1.0 | 0 |
| sKKVF18 | 1.0 ± 0.3 | 27.8 ± 3.0 | <1 | nd | >6 | nd | 0 | nd | 0 |
| sKKKVF18 | 2.3 ± 0.6 | 47.1 ± 8.1 | <1 | 1.3 | >6 | 2.1 | 2 | 2.5 | 1 |
| sKKKKVF18 | 1.9 ± 0.5 | 39.1 ± 5.4 | <1 | 1.7 | >6 | 2.9 | 6 | 3.2 | 1 |
| sRVF18 | 2.3 ± 0.6 | 53.6 ± 3.8 | <1 | 0.7 | >6 | 1.0 | 1 | 2.7 | 0 |
| sRRVF18 | 1.6 ± 0.8 | 49.1 ± 4.9 | <1 | 0.6 | >6 | 1.0 | 1 | 2.5 | 0 |
| sRRRVF18 | 2.6 ± 0.7 | 61.5 ± 6.2 | <1 | 1.2 | >6 | 2.2 | 6 | 2.4 | 1 |
| sRRRRVF18 | 2.3 ± 0.9 | 64.9 ± 11.4 | <1 | 1.1 | >6 | 1.8 | 6 | 2.0 | 1 |

*For peptides with IC₅₀ >10 μM, where the exact IC₅₀ is unknown, it was chosen to show the hemolytic activity at 50 μM.

### Example 3—Anti-Coagulative Properties of the Peptides

### Materials and methods

### Clotting Assay

Coagulometer (Amelung, Lemgo, Germany) was used to measure all clotting times. Freshly collected human citrated plasma was used for all experiment. To measure the activated partial thromboplastin time (aPTT), 100 μL of a kaolin-containing solution (Dapttin, Technocone) and plasma-peptide mix were incubated for 200 s at 37° C., then clot formation was initiated by adding 100 μL of 30 mM fresh CaCl₂ solution. Prothrombin clotting time (PT, thromboplastin reagent (Trinity Biotech)) was recorded by adding 100 μL clotting reagent to 100 μL pre-warmed (60 sec at 37° C.) plasma-peptide mix.

### Results

### Evaluation of Anti-Coagulative Properties of the Peptides

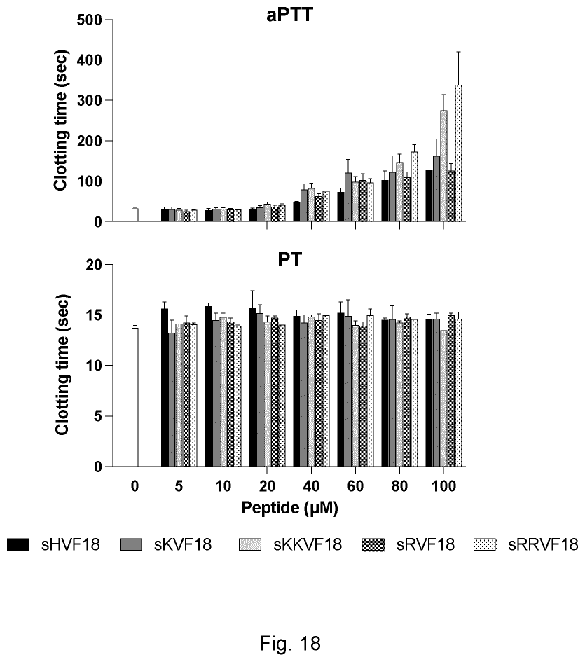

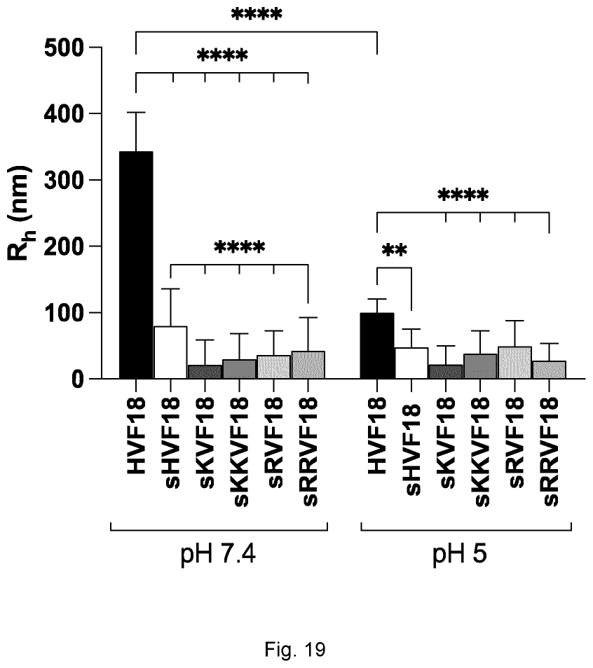

The peptides sHVF18, sKVF18, sKKVF18, sRVF18 and sRRVF18 were tested in the clotting assay. The sequences of these peptides are provided in the sequence overview below. The results are shown in FIG. 18.

Excessive activation of the clotting cascade via LPS and bacteria-induced contact activation contributes to the detrimental effects observed during sepsis and septic shock. Therefore, we investigated possible effects of the peptides of the invention on coagulation pathways. Analysis of peptide effects on the activated partial thromboplastin time (aPTT) and prothrombin time (PT) showed that albeit all peptide variants dose-dependently inhibited activation of the intrinsic pathway (aPTT) of coagulation in human plasma in vitro, the RR and KK variants sHVF18 were particularly effective (FIG. 18, upper panel). In contrast, the extrinsic pathway of coagulation, monitored by measuring the prothrombin time (PT) was not affected at any tested dose (FIG. 18, lower panel).

### Conclusion

Activation of coagulation, inhibition of fibrinolysis, and consumption of coagulation inhibitors lead to a procoagulant state resulting in fibrin deposition in the microvasculature as observed in ARDS and sepsis, diseases which may be complicated by disseminated intravascular coagulation (DIC). As a consequence, microvascular thrombosis contributes to promotion of organ dysfunction. Furthermore, excessive contact activation leads to the release of the pro-inflammatory peptide bradykinin and a subsequent induction of inflammatory reactions, which contribute to serious complications such as hypotension and vascular leakage. Therefore peptides, which in a biologically relevant context modulate several pathways, including inflammation and coagulation as demonstrated for sHVF18 and particularly the KK and RR variants, are of interest in developing future peptide-based treatments for patients presenting with an excessive activation of these pathways, such as seen in ARDS, sepsis and other systemic inflammatory disorders. Moreover, as activation of the contact system occurs in several non-infectious diseases, interference by the peptides may be beneficial in other conditions involving dysfunctional coagulation.

### Example 4—Oligomerization of the Peptides

### Material and Methods

### Dynamic Light Scattering (DLS)

The hydrodynamic radii of particles in solution were measured using a DynaPro Plate reader (WYATT Technology) equipped with a temperature-controlled chamber (25° C.). Peptides (HVF18, sHVF18, sKVF18, sKKVF18, sRVF18 and sRRVF18—sequences provided in the sequence overview below) were resuspended in 10 mM Tris at pH 7.4 or in 10 mM NaOAc at pH 5 at 1 mM as the final concentration, immediately before the analysis. 30 μL of each sample were used for the analysis. Each measurement was performed in triplicate with 10 sub-runs. The hydrodynamic radii were analyzed using Dynamics 7.19 Software. The results are expressed as mean values±DS obtained from three independent experiments.

### Results

### Oligomerization of the Peptides

The results are shown in FIG. 19. Oligomerization of the peptides is often considered a disadvantage since it is often associated with toxicity and immunogenicity, as well as a reduced activity. TCP-25 and HVF18 oligomerizes. The effect of stapling on oligomerization of the peptide was determined. The size of hydrodynamic radii (in nm) of the particles in solution was evaluated by dynamic light scattering (DLS), immediately after dissolving the peptides in 10 mM Tris at pH 7.4 or in 10 mM NaOAc at pH 5. We reported previously that at acidic pH the oligomerization of TCP-25 is inhibited. Moreover, at this pH the peptide was completely unstructured. The particles of HVF18 were larger at pH 7.4 than at pH 5. While sHVF18 showed particles with significantly reduced size compared to HVF18 at both pHs. When analyzing the hydrodynamic radii of K and R variants of sHVF18, the size of particles was even smaller and completely independent of pH, suggesting that both stapling and positive charge make the peptide less prone to oligomerize. Interestingly, no significant difference was observed between K and R variants as well as the number of these positively charged amino acids in the sequence.

### Example 5

### Materials and Methods

### Peptides

The peptide sHVF18 (HVFRLKKWIXKVIXQFGE)(SEQ ID NO: 3), its shorter versions (denoted as sVFR17, sFRL16, sRLK15, sLKK14, sKKW13), variants of sKKW13 (denoted as sKKK14, sKKK15, sRKK14, sRRK15), double stapled GKY25 (cyclo(GKYGFY)THVFRLKKWIXKVIXQFGE) denoted as 2sGKY25, were synthesised by AmbioPharm, Inc. (USA). Briefly, standard 9-fluorenylmethyloxycarbonyl (Fmoc) solid-phase peptide synthesis (SPSS) was used. To obtain hydrocarbon stapled peptides, olefin-bearing (S)-2-(4′pentenyl)-alanine was inserted at specified locations in the respective peptide sequences (denoted by X) in SEQ ID NOs: 3 and 13. Olefin metathesis reaction was performed on solid support using Grubbs' first-generation catalyst in 1,2-dichloroethane. The product peptides were cleaved from the resin and further purified by RP-HPLC. Peptides were provided as acetate salts, and the purity was confirmed with MALDI-TOF MS (>95%).

### Hemolysis Assay

The hemolytic effect of stapled peptides on erythrocytes and/or whole blood was tested as reported for sHVF18. Data shown are mean values±SEM obtained from at least four independent experiments all made in triplicate. For each experiment blood from a different donor was used.

### Whole Blood Assay

The anti-inflammatory effect of different stapled peptides in blood was performed as reported for sHVF18. For each experiment blood from a different donor was used.

### Cytokine Assay

Plasma obtained from blood experiments was used to evaluate cytokine release. Human inflammation DuoSet® ELISA Kit (R&D Systems) specific for TNF-α and IL-1β was used according to the manufacturer's instructions. Absorbance was measured at a wavelength of 450 nm. Data shown are mean values±SEM obtained from four independent experiments.

### Porcine ARDS Model

Animal Preparation: Female and male adult farm-raised wild-type American Yorkshire pigs (Sus scrofa domesticus) were included in the study. The animals were stratified into either treatment or non-treatment groups. A total of 10 pigs with a mean weight of 45 kg were premedicated with ketamine (Ketaminol® vet. 100 mg/mL; Farmaceutici Gellini S.p.A., Aprilia, Italy; 20 mg kg⁻¹) and xylazine (Rompun® vet 20 mg mL⁻¹; Bayer AG, Leverkusen, Germany; 2 mg kg⁻¹). A urinary catheter was inserted into the bladder. A peripheral intravenous (IV) line was placed in the earlobe and general anesthesia was maintained with ketamine (Ketaminol® vet, MSD Animal Health Sweden, Stockholm, Sweden), midazolam (Midazolam Panpharma®, Panpharma Nordics AS, Oslo, Norway) and fentanyl (Leptanal®, Piramal Critical Care B.V., Voorschoten, Netherlands) infusions. A Siemens-Elema ventilator (Servo 900C, Siemens, Solna, Sweden) was used to establish mechanical ventilation with a 7.5 size endotracheal tube for intubation. Volume-controlled ventilation (VCV) with the flow pattern switch in “constant flow” was used which lowers the peak pressures according to the manufacturer's instructions. To achieve an I:E ratio of 1:2, inspiration time was set to 25% with a pause time of 10%, and ventilation was adjusted to maintain carbon dioxide levels (PaCO₂) between 33-41 mmHg. The tidal volume (Vt) was kept at 6-8 mL kg⁻¹. The equation (a) was used to determine the dynamic compliance.

$${C}_{dyn} = \frac{{V}_{T}}{( peak pressure - PEEP )} \qquad ( a )$$

Further, an arterial line (Secalon-T™, Merit Medical Ireland Ltd, Galway, Ireland) was inserted in the right common carotid artery. A pulmonary artery catheter (Swan-Ganz CCOmbo V and Introflex, Edwards Lifesciences Services GmbH, Unterschleissheim, Germany) was placed in the right internal jugular vein.

ARDS-induction with Lipopolysaccharide: To induce an ARDS according to the Berlin criteria (Force et al.) E. coli LPS (O111:B4, Sigma-Aldrich, Merck KGaA, Darmstadt, Germany) was used intravenously. Prior to administration LPS was diluted in saline solution (2 μg kg⁻¹ min⁻¹). As a result of the LPS administration, all animals developed hemodynamic instability and required continuous inotropic support, provided as an infusion of norepinephrine (40 μg mL⁻¹, 0.05-2 μg kg⁻¹ min⁻¹; Pfizer AB, Sollentuna, Sweden) and dobutamine (2 mg mL⁻¹, 2.5-5 μg kg⁻¹ min⁻¹; Hameln Pharma Plus GmbH, Hameln, Germany). Ringer's acetate (Baxter Medical AB, Kista, Sweden) was used to compensate for fluid loss. The different ARDS stages were defined based on the measured PaO₂ FiO₂⁻¹ ratio, according to the Berlin definition (Force et al.): Mild ARDS for a ratio between 201-300 mmHg, moderate ARDS for a ratio between 101-200 mmHg, and severe ARDS for a ratio s 100 mmHg. The ARDS state was considered as confirmed, when two separate arterial blood gas measurements, taken within a 15-minute interval, fell within the Berlin definition's PaO₂ FiO₂⁻¹ range.

sHVF18 Treatment: In the treated cohort, each animal received two intravenous doses of peptide solution (12 mg in 50 mL) over the course of 30 minutes, administrated using the central venous catheter in the superior vena cava.

Arterial blood gas analysis: Arterial blood was collected every 30 minutes and analyzed with an ABL 90 FLEX blood gas analyzer (Radiometer Medical ApS, Brønshøj, Denmark). According to clinical standards, the measurements were normalized to a blood temperature of 37° C.

Hemodynamic measurements: The animals were closely observed, hemodynamic parameters were measured and recorded before the start of ARDS-induction and every 30 minutes thereafter, using thermodilution with a Swan-Ganz catheter and an arterial line. Parameters recorded were heart rate (HR), systolic blood pressure (SBP), diastolic blood pressure (DBP), mean arterial pressure (MAP), central venous pressure (CVP), cardiac output (CO), systolic pulmonary pressure (SPP), diastolic pulmonary pressure (DPP), mean pulmonary pressure (MPP), pulmonary artery wedge pressure (PAWP), systemic vascular resistance (SVR), and pulmonary vascular resistance (PVR).

Histopathological Analyses: Biopsies of the right lower lobe were taken after confirmed ARDS. Biopsies were immediately transferred in 10% neutral buffered formalin solution (Sigma Aldrich, St. Louis, Missouri, USA) and fixed at 4° C. overnight. Formalin-fixed tissues were processed with a graded ethanol series (solutions obtained from Histolab Products AB, Gothenburg, Sweden) and clearing solution (Sigma Aldrich) prior to paraffin embedding (Histolab). Sections of 4 μm were cut and transferred to SuperFrost Plus microscopy slides (Thermo Fisher Scientific, Waltham, Massachusetts, US). Sections were dried at room temperature overnight. Sections were stained with hematoxylin and eosin (both Merck Millipore, Darmstadt, Germany) after de-paraffinization, followed by dehydration in consecutively graded ethanol and xylene solutions (Histolab). Stained sections were finally mounted with Pertex solution (Histolab). Bright-field images were acquired with an Olympus CKX53 microscope (Olympus, Shinjuku, Tokyo, Japan). Images from each animal were scored independently for lung injury by three blinded scorers on the basis of five parameters: number of immune cells in alveolar space and in interstitial space, occurrence of proteinaceous debris, presentation of alveolar septal thickening and structural changes, and lastly presentation of hemorrhage, hyaline membranes, or other signs of enhanced injury. Scores were given based on a scale of 0 to 6 for each criterium. The scores are presented as the average of the sum of the characteristic scores for each sample.

### Results

Analysis of the Hemolytic and Anti-Inflammatory Activity of Shorter Variants of sHVF18

As shown in Example 1 stapling of HVF18 increased its stability to proteolysis and greatly improved its anti-inflammatory activity (in vitro and in vivo), retaining its antimicrobial activity, but turning it mostly against Gram-positive bacteria, while at the same time having a low hemolytic activity. Shorter variants of sHVF18 were made, to evaluate how small can be the active peptide. Double stapled GKY25 (2sGKY25), i.e. one in the C-terminal region as for sGKY25, and one in the N-terminal region, was used for comparison.

The library of different stapled peptides was generated and then screened for their hemolytic effect (FIG. 20a) versus anti-inflammatory activity (FIG. 20b). Reporting the percentage of hemolytic activity of all peptides on RBCs in function of the concentration of the same peptide needed to have IC₅₀ of TNF-α and IL-1β in LPS-stimulated blood, emerged that in particular sHVF18 was promising (FIG. 20c).

Analysis of the Hemolytic and Anti-Inflammatory Activity of sKKW13 Variants

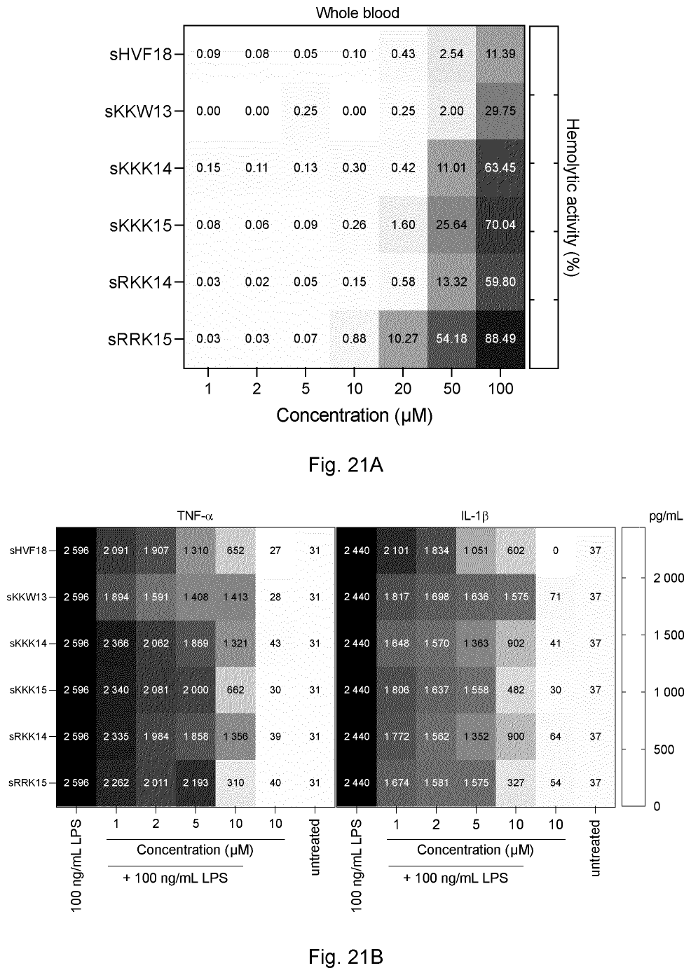

Both sLKK14 and sKKW13 showed low hemolytic activity (FIG. 20a), but sLKK14 was was prone to oligomerize once in solution and concomitantly losing its immunomodulatory effect, while sKKW13 was not (FIG. 20b). Therefore, we hypothesized that adding one or two K and R residues to sKKW13 could have improved its anti-inflammatory activity. We first compared the hemolytic activity of these new peptides to the original one and to the sHVF18, on whole blood (FIG. 21a). The K and R variants showed much higher hemolytic activity. Then we tested their ability to reduce cytokines production in LPS-stimulated blood (FIG. 21b). All the K and R variants were less active than sHVF18 and no big improvement was obtained if compared to sKKW13.

sHVF18 Effects in a Porcine ARDS Model

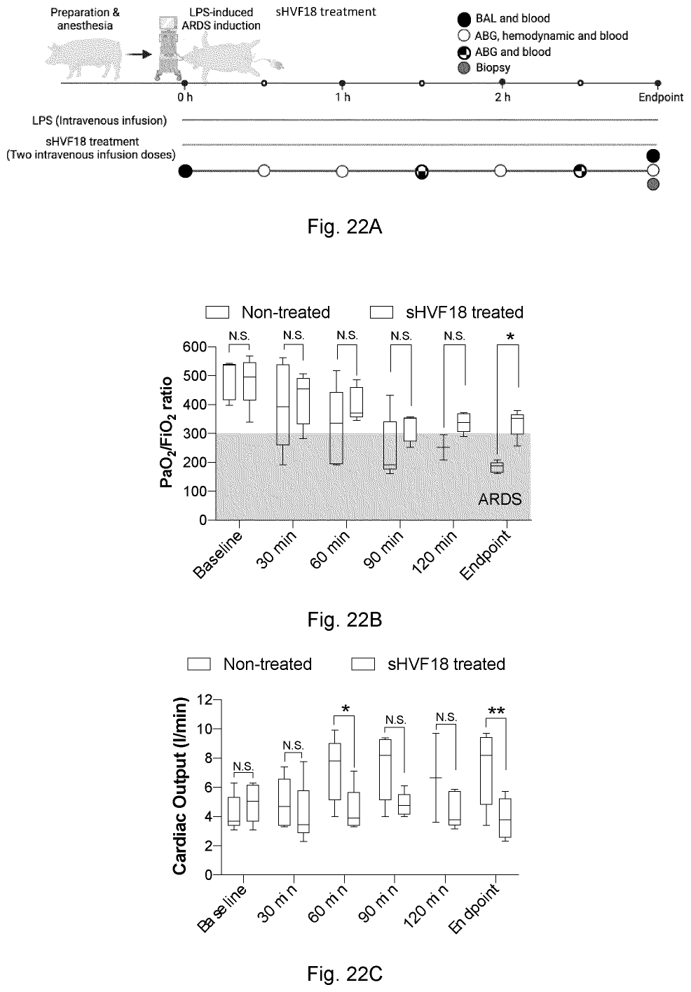

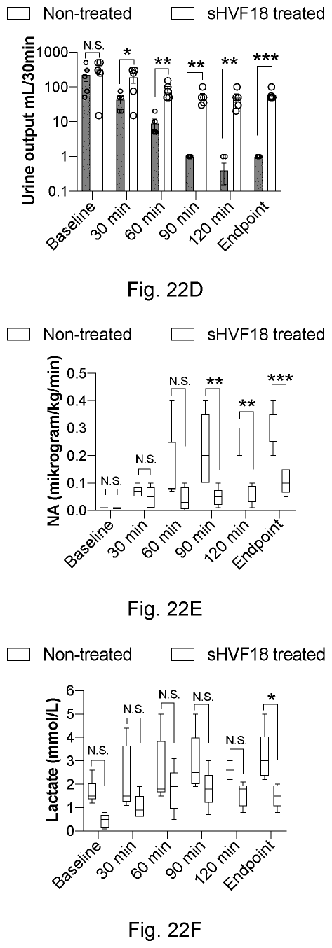

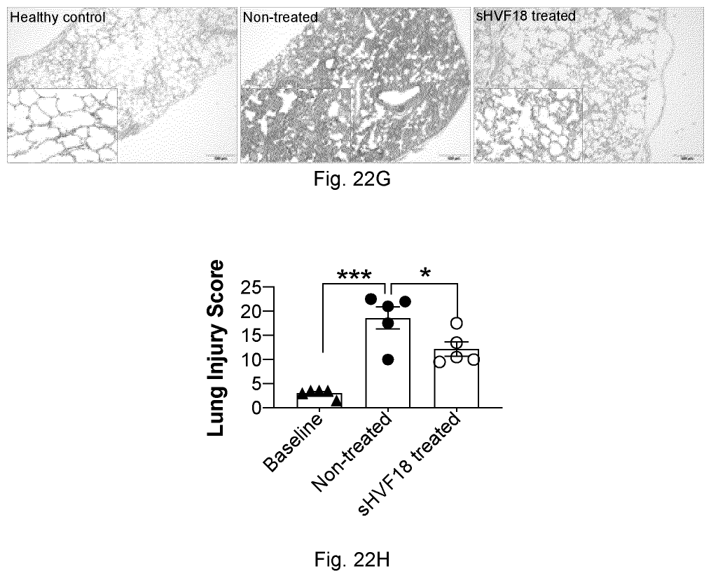

We further investigated therapeutic effects of sHVF18 in an established preclinical porcine model of ARDS. ARDS was induced injecting intravenously E. coli LPS (study outlines are presented in FIG. 22a). All pigs developed hemodynamic instability and requiring inotropic support with norepinephrine following LPS administration (inotropic support refers to the use of agents of dobutamine, and norepinephrine with the clinical purpose of maintaining hemodynamic stability). This hemodynamic instability, but also the differences between treated and not treated pigs over the time course of the experiment, is shown by PaO₂/FiO₂ ratio (FIG. 22b), cardiac output (FIG. 22c), urine output (FIG. 22d), norepinephrine (NA, FIG. 22e), and lactate levels (FIG. 22f).

Overall, following intravenous administration of sHVF18 the treated animals stabilized in their hemodynamics and required significant less inotropic support compared to the not treated animals. The treated animals did not deteriorate as much as the not treated animals in PaO₂/FiO₂ ratio and only one of the five treated animals had mild ARDS according to the blood gases (according to Berlin definition of ARDS).

At the endpoint of the experiment lung tissue samples were taken from right lower lobe and were compared to lung tissue samples from five healthy control pigs. Lung biopsy taken from healthy controls for histological analysis appeared normal, with no anomalies (FIG. 22g, left panel). All biopsies taken from both treated and not treated animals at the endpoint of the experiment showed infiltration of immune cells and signs of diffuse alveolar damage, including thickening of the alveolar capillary barrier with intra-alveolar haemorrhage, however less in the treated pigs (FIG. 22g, middle and right panels). In addition to subjective analysis of lung histology, blinded scoring was performed on all pigs by three independent observers. Significant increases in a cumulative lung injury score in the not treated group compared to healthy controls were seen, accounting for multiple signs of lung injury following acute lung injury onset via LPS administration. Significant differences were seen between the treated and non-treated pigs (FIG. 22h) with less signs of lung injury in the treated pigs.

### Example 6

### Materials and Methods

### In Silico Analysis of Peptide Staple Positions

The NMR structure of HVF18 (PDB: 5Z5X) (Saravanan et al., 2018) was docked to the modelled structure of human CD14 using the ClusPro webserver (Kozakov et al., 2017). Similar results were obtained as described in Saravanan et al., 2018, whereby the peptide binds to the N-terminus of CD14. The structure of the top scoring docking pose was selected as a template to model GKY25. The N-terminal GKYGFYT residues were modelled using Modeller version 9.21 and the model with the lowest discrete optimised protein energy score (Shen et al., 2006) was chosen. The GKY25-CD14 complex was solvated with TIP3P water and NaCl salts using the CHARMM-GUI Solution Builder as described in (Jo et al., 2008). A steepest descent energy minimisation and a 125 ps equilibration simulation, whereby positional restraints were applied to the backbone atoms of the protein and peptide, was performed following the standard CHARMM-GUI protocols (Lee et al., 2016). The final snapshot after equilibration was extracted and the binding energy between GKY25 and CD14 was calculated using the Molecular mechanics Poisson-Boltzmann surface area (MMPBSA) method (Kumari et al., 2014). A hydrophobic staple was added to the GKY25 peptide by mutating residues at positions i and i+3 to alanine and linking them with two pentene segments using the CHARMM-GUI Solution Builder (Jo et al., 2008). Stapled GKY25 bound to CD14 were then subjected to the same solvation, minimization and equilibration procedures described above, after which their binding energies were determined using MMPBSA. A similar protocol was performed for the addition of a staple at positions i and i+4. The difference in binding energies to the non-stapled GKY25 was then calculated. A similar analysis of peptide staple positions was also performed on the shorter HVF18 peptide.

### Results

The Design of Dual-Action Peptides with Improved Proteolytic Stability.

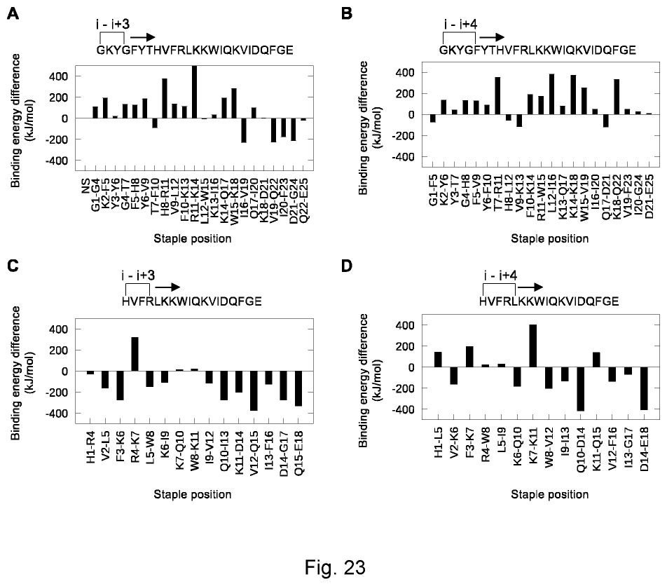

We then performed an in silico analysis of peptide staple positions, whereby a short hydrophobic pentenyl-alanine staple linking either residues i and i+3 or residues i and i+4 was added along the sequence of GKY25. To determine the effect of adding this staple on binding to CD14, we then calculated the difference in binding energies between non-stapled and stapled version of the peptide and the results are shown in FIG. 23. We found that the addition of staple at most positions in the longer GKY25 peptide, particularly on the N-terminal region of the peptide, resulted in poorer binding as demonstrated by the positive binding energy differences (see results in FIG. 23 A and B). The reduced affinities could be caused by the staple perturbing interactions between the peptide and CD14. Some staples resulted in improved binding to CD14. For the i−i+3 configuration, these include I16-V19, V19-Q22, I20-D23, and D21-G24, whereas for the i−i+4 configuration, these include V9-K13 and Q17-D21. All the staples that resulted in a more favourable binding to CD14 comprises hydrophobic residues, which could be important for interaction with LPS, except the Q17-D21 staple.

In contrast, for the shorter HVF18 peptide, improved affinity was observed for most stables (see FIG. 23C and D). For the i−i+3 configuration at least the following staples resulted in improved affinity: V2-L5, F3-K6, L5-W8, K6-I9, I9-V12, Q10-I13, K11-D14, V12-Q15, I13-F16, D14-G17 and Q15-E18. For the i−i+4 configuration, at least the following staples resulted in improved affinity: V2-K6, K6-Q10, W8-V12, I9-I13, Q10-D14, V12-F16, I13-G17 and D14-E18. For the staples that resulted in improved binding, the Q10-D14 (equivalent to Q17-D21 in GKY25) has one of the most negative binding energy differences.

### REFERENCES

Force, A. D. T. et al. Acute respiratory distress syndrome: the Berlin Definition. JAMA 307, 2526-2533 (2012).
Jo, S., Kim, T., Iyer, V. & Im, W. CHARMM-GUI: A Web-Based Graphical User Interface for CHARMM. J. Comput. Chem. 29, 1859-1865 (2008).
Kalle, M. et al. Host defense peptides of thrombin modulate inflammation and coagulation in endotoxin-mediated shock and Pseudomonas aeruginosa sepsis. PLoS One 7, e51313 (2012).
Kozakov, D. et al. The ClusPro web server for protein-protein docking. Nat. Protoc. 12, 255-278 (2017).
Kumari, R., Kumar, R. & Lynn, A. G-mmpbsa—A GROMACS tool for high-throughput MM-PBSA calculations. J. Chem. Inf. Model. 54, 1951-1962 (2014).
Lee, J. et al. CHARMM-GUI Input Generator for NAMD, GROMACS, AMBER, OpenMM, and CHARMM/OpenMM Simulations Using the CHARMM36 Additive Force Field. J. Chem. Theory Comput. 12, 405-413 (2016).
Li, X., et al. Stapled Helical Peptides Bearing Different Anchoring Residues, Chem. Rev. 2020, 120, 10079-10144
Morrisett, J. D., et al. Interaction of an apolipoprotein (apoLP-alanine) with phosphatidylcholine. Biochemistry 12, 1290-1299 (1973).
Papareddy, P. et al. Proteolysis of human thrombin generates novel host defense peptides. PLoS Pathog 6, e1000857 (2010).
Petruk, G., et al. SARS-CoV-2 Spike protein binds to bacterial lipopolysaccharide and boosts proinflammatory activity. Journal of Molecular Cell Biology, 12(12) 916-932 (2020).
Petruk, G., et al., Concentration- and pH-dependent oligomerization of the thrombin-derived C-terminal peptide TCP-25. Biomolecules, 10(11), 1572 (2020).
Puthia, M. et al. A dual-action peptide-containing hydrogel targets wound infection and inflammation. Sci Transl Med12 (2020).
Puthia, M., et al. A dual-action peptide-containing hydrogel targets wound infection and inflammation. Science translational medicine, 12(524) (2020).
Saravanan, R., et al. Structural basis for endotoxin neutralisation and anti-inflammatory activity of thrombin-derived C-terminal peptides. Nature communications, 9(1), 1-14 (2018).
Shen, M., Devos, D., Melo, F. & Sali, A. A composite score for predicting errors in protein structure models. Protein Sci. 15, 1653-1666 (2006).
Shi, X. E. et al. Hydrogen exchange-mass spectrometry measures stapled peptide conformational dynamics and predicts pharmacokinetic properties. Anal Chem 85, 11185-11188 (2013).
Stromdahl, A. C. et al. Peptide-coated polyurethane material reduces wound infection and inflammation. Acta Biomater (2021).

IVEGSDAEIGMSPWQVMLFRKSPQELLCGASLISDRWVLTAAHCL
LYPPWDKNFTENDLLVRIGKHSRTRYERNIEKISMLEKIYIHPRY
NWRENLDRDIALMKLKKPVAFSDYIHPVCLPDRETAASLLQAGYK
GRVTGWGNLKETWTANVGKGQPSVLQVVNLPIVERPVCKDSTRIR
ITDNMFCAGYKPDEGKRGDACEGDSGGPFVMKSPFNNRWYQMGIV
SWGEGCDRDGKYGFYTHVFRLKKWIQKVIDQFGE

### Abbreviations

AMPs, antimicrobial peptides; AMR, antimicrobial resistance; CD, circular dichroism; PAMPs, pathogen-associated molecular patterns; TCP-25, Thrombin C-terminal Peptide of 25 aminoacids; TEM, Transmission electron microscopy; TLRs, Toll-like receptors.

### Sequence Overview

| SEQ | | |
|---|---|---|
| ID | | |
| NO | Description | Sequence |
| 1 | Thrombin | IVEGSDAEIGMSPWQVMLFRKSPQELLCGASLISDRW |
| | (peptide GKY25 is | VLTAAHCLLYPPWDKNFTENDLLVRIGKHSRTRYERN |
| | indicated in bold) | IEKISMLEKIYIHPRYNWRENLDRDIALMKLKKPVAF |
| | | SDYIHPVCLPDRETAASLLQAGYKGRVTGWGNLKETW |
| | | TANVGKGQPSVLQVVNLPIVERPVCKDSTRIRITDNM |
| | | FCAGYKPDEGKRGDACEGDSGGPFVMKSPFNNRWYQM |
| | | GIVSWGEGCDRDGKYGFYTHVFRLKKWIQKVIDQFGE |
| 2 | HVF18 | HVFRLKKWIQKVIDQFGE |
| 3 | SHVF18 | HVFRLKKWIX₁KVIX₂QFGE |
| 4 | SKVF18 | KVFRLKKWIX₁KVIX₂QFGE |
| 5 | SKKVF18 | KKVFRLKKWIX₁KVIX₂QFGE |
| 6 | sKKKVF18 | KKKVFRLKKWIX₁KVIX₂QFGE |
| 7 | sKKKKVF18 | KKKKVFRLKKWIX₁KVIX₂QFGE |
| 8 | sRVF18 | RVFRLKKWIX₁KVIX₂QFGE |
| 9 | SRRVF18 | RRVFRLKKWIX₁KVIX₂QFGE |
| 10 | sRRRVF18 | RRRVFRLKKWIX₁KVIX₂QFGE |
| 11 | sRRRRVF18 | RRRRVFRLKKWIX₁KVIX₂QFGE |
| 12 | GKY25 | GKYGFYTHVFRLKKWIQKVIDQFGE |
| 13 | SGKY25 | GKYGFYTHVFRLKKWIX₁KVIX₂QFGE |
| 14 | 2sGKY25 | cyclo[GKYGE]YTHVFRLKKWIX₁KVIX₂QFGE |
| 15 | FYT21 | FYTHVFRLKKWIQKVIDQFGE |
| 16 | Prothrombin | MAHVRGLQLPGCLALAALCSLVHSQHVFLAPQQARS |
| | (peptide GKY25 is | LLQRVRRANTFLEEVRKGNLERECVEETCSYEEAFE |
| | indicated in bold, | ALESSTATDVFWAKYTACETARTPRDKLAACLEGNC |
| | pro-peptide | AEGLGTNYRGHVNITRSGIECQLWRSRYPHKPEINS |
| | underlined) | TTHPGADLQENFCRNPDSSTTGPWCYTTDPTVRRQE |
| | | CSIPVCGQDQVTVAMTPRSEGSSVNLSPPLEQCVPD |
| | | RGQQYQGRLAVTTHGLPCLAWASAQAKALSKHQDFN |
| | | SAVQLVENFCRNPDGDEEGVWCYVAGKPGDFGYCDL |
| | | NYCEEAVEEETGDGLDEDSDRAIEGRTATSEYQTFF |
| | | NPRTFGSGEADCGLRPLFEKKSLEDKTERELLESYI |
| | | DGRIVEGSDAEIGMSPWQVMLFRKSPQELLCGASLI |
| | | SDRWVLTAAHCLLYPPWDKNFTENDLLVRIGKHSRT |
| | | RYERNIEKISMLEKIYIHPRYNWRENLDRDIALMKL |
| | | KKPVAFSDYIHPVCLPDRETAASLLQAGYKGRVTGW |
| | | GNLKETWTANVGKGQPSVLQVVNLPIVERPVCKDST |
| | | RIRITDNMFCAGYKPDEGKRGDACEGDSGGPFVMKS |
| | | PFNNRWYQMGIVSWGEGCDRDGKYGFYTHVFRLKKW |
| | | IQKVIDQFGE |
| 17 | SVFR17 | VFRLKKWIX₁KVIX₂QFGE |
| 18 | SFRL16 | FRLKKWIX₁KVIX₂QFGE |
| 19 | SRLK15 | RLKKWIX₁KVIX₂QFGE |
| 20 | SLKK14 | LKKWIX₁KVIX₂QFGE |
| 21 | SKKW13 | KKWIX₁KVIX₂QFGE |
| 22 | SKKK14 | KKKWIX₁KVIX₂QFGE |
| 23 | SKKK15 | KKKKWIX₁KVIX₂QFGE |
| 24 | sRKK14 | RKKWIX₁KVIX₂QFGE |
| 25 | SRRK15 | RRKKWIX₁KVIX₂QFGE |
| 26 | 2sGKY25_2 | cyclo[X₃KYGX₄]YTHVFRLKKWIX₁KVIX₂QFGE |

In the sequence list above, X₁ and X₂ are each initially (S)-2-(4′pentenyl)-alanine, which are reacted with each other by RCM to form an olefin tether. In the sequence listing, the sequences of the unreacted peptides are given. The skilled person will understand that even though the unreacted sequences are provided, in general, the peptides are used in the stapled format, i.e. after the olefin tether has been formed by RCM. Further, X₃ and X₄ are glycine and glutamic acid, respectively, which are reacted with each other to form a covalent bond in the form of a lactam bridge.

### Items

The invention may further be defined by any one of the following items:

1. A peptide comprising a consecutive sequence of in the range of 10 to 23 amino acids from thrombin of SEQ ID NO: 1 containing up to 6 amino acid substitutions, wherein said peptide:
i) has a total length between 10 and 40 amino acids;
ii) comprises at least one internal covalent linkage between the side chains of two non-neighbouring, internal amino acids, wherein said amino acids are denoted X₁ and X₂; and
iii) comprises at least amino acids K247, K248 and K252 of thrombin of SEQ ID NO: 1;
   with the proviso that if the peptide has a total length between 24 to 40 amino acids it comprises at least two internal covalent linkages between the side chains of two non-neighbouring, internal amino acids, wherein the amino acids of the first internal covalent linkage are denoted X₁ and X₂, and the amino acids of the second internal covalent linkage are denoted X₃ and X₄.

2. The peptide according to item 1, wherein the peptide comprises a consecutive sequence in the range of 10 to 40 amino acids from thrombin of SEQ ID NO: 1.

3. A peptide comprising a consecutive sequence of in the range of 10 to 23 amino acids from thrombin of SEQ ID NO: 1 containing up to 6 amino acid substitutions, wherein said peptide:
i) has a total length between 10 and 23 amino acids;
ii) comprises at least one internal covalent linkage between the side chains of two non-neighbouring, internal amino acids, wherein said amino acids are denoted X₁ and X₂; and
iii) comprises at least amino acid K247, K248 and K252 of thrombin of SEQ ID NO: 1.

4. A peptide comprising a consecutive sequence of in the range of 10 to 23 amino acids from GKY25 of SEQ ID NO: 12 containing up to 6 amino acid substitutions, wherein said peptide:
i) has a total length between 10 and 23 amino acids;
ii) comprises at least one internal covalent linkage between the side chains of two non-neighbouring, internal amino acids, wherein said amino acids are denoted X₁ and X₂; and
iii) comprises at least amino acids K13, K14 and K18 of GKY25 of SEQ ID NO: 12.

5. The peptide according to any one of the preceding items, wherein peptide comprises a consecutive sequence of in the range of 13 to 23 amino acids from thrombin of SEQ ID NO: 1 or GKY25 of SEQ ID NO: 12.

6. A peptide comprising or consisting of the amino acid sequence:

-U-U-(Z)ₙ-I-Q-K-V-I-D-Q-(Z)ₘ-

wherein
the peptide has a total length between 10 to 23 amino acids; and
each Z is individually any canonical amino acid; and
U is His, Lys or Arg; and
n is an integer in the range of 0 to 10; and
m is an integer in the range of 0 to 5, and
wherein two of the amino acids have been substituted for alkenylated amino acids, the side chains of which are linked by a covalent linkage.

7. The peptide according to any one of the preceding items, wherein the peptide has a total length of 13 to 23 amino acids.

8. A peptide comprising a consecutive sequence of in the range of 13 to 23 amino acids from thrombin of SEQ ID NO: 1 containing up to 6 amino acid substitutions, wherein said peptide:
i) has a total length between 13 and 23 amino acids;
ii) comprises at least one internal covalent linkage between the side chains of two non-neighbouring, internal amino acids, wherein said amino acids are denoted X₁ and X₂; and
iii) comprises at least amino acids R245, K247, K248, K252 of thrombin of SEQ ID NO: 1.

9. The peptide according to any one of items 1 and 8, wherein the peptide comprises at least amino acids R245, K247, K248, K252 of thrombin of SEQ ID NO: 1.

10. A peptide comprising a consecutive sequence of in the range of 13 to 23 amino acids from GKY25 of SEQ ID NO: 12 containing up to 6 amino acid substitutions, wherein said peptide:
i) has a total length between 13 and 23 amino acids;
ii) comprises at least one internal covalent linkage between the side chains of two non-neighbouring, internal amino acids, wherein said amino acids are denoted X₁ and X₂; and
iii) comprises at least amino acids K13, K14 and K18 of GKY25 of SEQ ID NO: 12.

11. The peptide according to item 10, wherein the peptide comprises at least amino acids R11, K13, K14 and K18 of thrombin of SEQ ID NO: 12.

12. The peptide according to any one of the preceding items, wherein the peptide contains only one internal covalent linkage between two non-neighbouring, internal amino acids.

13. The peptide according to any one of the preceding items, wherein amino acid X₁ is positioned at position n and amino acid X₂ is positioned at position n+3, or at position, n+4, or at position n+5, or at position n+6, or at position n+7, or at position n+8, or at position n+9, or at position n+10, or at position n+11, wherein n is an integer in the range of 2 to 18.

14. The peptide according to any one of the preceding items, wherein amino acid X₁ is positioned at position n and amino acid X₂ is positioned at position n+3, or at position n+4, or at position n+7, or at position n+11, wherein n is an integer in the range of 2 to 18.

15. The peptide according to any one of the preceding items, wherein amino acid X₁ is positioned at position n and amino acid X₂ is positioned at position at position n+3 or at position n+4, or at position n+7, wherein n is an integer in the range of 2 to 18.

16. The peptide according to any one of the preceding items, wherein amino acid X₁ is positioned at position n and amino acid X₂ is positioned at position at position n+4, or at position n+7, wherein n is an integer in the range of 2 to 18.

17. The peptide according to any one of the preceding items, with the proviso that when aligning the peptide sequence to the sequence of GKY25 of SEQ ID NO: 12, then
a. X₂ does not align to a Lys in GKY25 of SEQ ID NO: 12; and
b. X₂ does not align to a Gln, when X₁ aligns to a Lys.

18. The peptide according to any one of the preceding items, with the proviso that when aligning the peptide sequence to the sequence of GKY25 of SEQ ID NO: 12, then
a. X₁ does not align to Arg11 in GKY25 of SEQ ID NO: 12
b. X₁ does not align to Lys14 in GKY25 of SEQ ID NO: 12
c. X₂ does not align to Lys14 in GKY25 of SEQ ID NO: 12; and
d. X₂ does not align to Lys 18 in GKY25 of SEQ ID NO: 12; and
e. X₂ does not align to Gln22, when X₁ aligns to Lys18 of SEQ ID NO: 12.

19. The peptide according to any one of the preceding items, wherein amino acid X₁ is positioned at position n and amino acid X₂ is positioned at position at position n+3, wherein n is an integer in the range of 2 to 18, with the proviso that when aligning the peptide sequence to the sequence of GKY25 of SEQ ID NO: 12, then
a. X₁ does not align to Arg11 in GKY25 of SEQ ID NO: 12
b. X₁ does not align to Lys14 in GKY25 of SEQ ID NO: 12; and
c. X₂ does not align to Lys14 in GKY25 of SEQ ID NO: 12.

20. The peptide according to any one of the preceding items, wherein amino acid X₁ is positioned at position n and amino acid X₂ is positioned at position at position n+4, wherein n is an integer in the range of 2 to 18, with the proviso that when aligning the peptide sequence to the sequence of GKY25 of SEQ ID NO: 12, then
a. X₁ does not align to Arg11 in GKY25 of SEQ ID NO: 12
b. X₁ does not align to Leu12 in GKY25 of SEQ ID NO: 12
c. X₁ does not align to Lys14 in GKY25 of SEQ ID NO: 12
d. X₂ does not align to Lys14 in GKY25 of SEQ ID NO: 12;
e. X₁ does not align to Lys 18 in GKY25 of SEQ ID NO: 12; and
f. X₂ does not align to Lys 18 in GKY25 of SEQ ID NO: 12.

21. The peptide according to any one of the preceding items, wherein amino acid X₁ is positioned at position n and amino acid X₂ is positioned at position at position n+3, wherein n is an integer in the range of 2 to 18, wherein when aligning the peptide sequence to the sequence of GKY25 of SEQ ID NO: 12, then X₁ and X₂ corresponds to Val9 and Leu12; or Phe10 and Lys13; or Leu12 and Trp15; or Lys13 and Ile16, or Ile16 and Val19; or Gln17 and Ile20; or Lys18 and Asp21; or Val19 and Gln22; or Ile20 and Phe23; or Asp21 and Gly24; or Gln22 and Glu25 of SEQ ID NO: 12.

22. The peptide according to any one of the preceding items, wherein amino acid X₁ is positioned at position n and amino acid X₂ is positioned at position at position n+4, wherein n is an integer in the range of 2 to 18, wherein when aligning the peptide sequence to the sequence of GKY25 of SEQ ID NO: 12, then X₁ and X₂ corresponds Val9 and Lys13; or Lys13 and Gln17; or Trp15 and Val19; or Ile16 and Ile20; or Gln17 and Asp21; or Val19 and Phe23; or Ile20 and Gly24; or Asp21 and Glu18 of SEQ ID NO: 12.

23. The peptide according to any one of the preceding items, wherein amino acid X₁ is positioned at position n and amino acid X₂ is positioned at position at position n+4, wherein n is an integer in the range of 2 to 18, wherein when aligning the peptide sequence to the sequence of GKY25 of SEQ ID NO: 12, then X₁ and X₂ corresponds Gln17 and Asp21 of SEQ ID NO: 12.

24. The peptide according to any one of the preceding items, wherein X₁ and X₂ are selected from the group consisting of
i) X₁ is Lys and X₂ is selected from the group consisting of Asp, Glu, Lys, Cys and Tyr;
ii) X₁ is Cys and X₂ is selected from the group consisting of Cys, Lys and Met;
iii) X₁ is Asp and X₂ is Lys;
iv) X₁ is Glu is X₂ is selected from the group consisting of Lys and Glu;
v) X₁ is Tyr and X₂ is selected from the group consisting of Lys, Phe and Trp;
vi) X₁ is Met and X₂ is selected from the group consisting of Met and Cys;
vii) X₁ is His and X₂ is His;
viii) X₁ is Phe and X₂ is selected from the group consisting of Phe, Tyr, Ala and Trp;
ix) X₁ is Ala and X₂ is Phe or Tyr;
x) X₁ is Trp and X₂ is selected from the group consisting of Trp, Phe and Tyr;
xi) X₁ is Gly and X₂ is Glu.

25. The peptide according to any one of the preceding items, wherein X₁ is Lys and X₂ is Asp, Glu, Cys or Lys or vice versa.

26. The peptide according to any one of the preceding items, wherein X₁ and X₂ are Cys, and wherein the covalent linkage is either a direct covalent bond (i.e. a disulphide bridge) or via a crosslinker, wherein the crosslinker for example is a bis-alkylator, such as linker comprising at least two (bromomethyl) substituents.

27. The peptide according to any one of the preceding items, wherein either X₁ or X₂ is a derivatised canonical amino acid, for example selected from the group consisting of Ser derivatives and Ala derivatives.

28. The peptide according to any one of the preceding items, wherein the covalent linkage is formed by linking two non-canonical amino acids, optionally wherein said non-canonical amino acids have substituted two native amino acids of thrombin.

29. The peptide according to any one of the preceding items, wherein the covalent linkage is a hydrocarbon staple.

30. The peptide according to any one of the preceding items, wherein X₁ and X₂ are alkenylated amino acids, such as two C-alkenylated amino acids, such as two α-substituted alkenyl amino acids and/or α,α-disubstituted alkenyl amino acids, and the covalent linkage is an olefin tether formed between said alkenyl residues.

31. The peptide according to item 30, wherein said alkenylated amino acids are amino acids native to thrombin, which have been alkenylated, and/or alkenylated amino acids substituting amino acids native to thrombin.

32. The peptide according to any one of the preceding items, wherein X₁ and X₂ individually are selected from the group consisting of alkenylated Ala, alkenylated Leu, alkenylated Met, alkenylated Ser, alkenylated Tyr, alkenylated Lys, alkenylated Arg and alkenylated Phe.

33. The peptide according to any one of the preceding items, wherein either X₁ or X₂ is alkenylated Ala and the other is selected from the group consisting of alkenylated Ala, alkenylated Leu, alkenylated Met, alkenylated Ser, alkenylated Tyr, alkenylated Lys, alkenylated Arg and alkenylated Phe.

34. The peptide according to any one of the preceding items, wherein X₁ and/or X₂ are alkenylated alanine.

35. The peptide according to any one of the preceding items, wherein X₁ and/or X₂ are α,α-disubstituted alkenylated alanine, and the covalent linkage is an olefin tether formed between said alkenyl residues.

36. The peptide according to any one of the preceding items, wherein X₁ and/or X₂ are linked by a tether, wherein the tether is an alkene chain of 10 carbon atoms counting from the C-alpha carbon.

37. The peptide according to any one of the preceding items, wherein X₁ and/or X₂ are alkenylated alanines, which have been linked to each other by ring closing metathesis forming an alkene tether, wherein the tether is an alkene chain of 10 carbon atoms counting from the C-alpha carbon.

38. The peptide according to any one of items 36 to 37, wherein said alkene chain is an unbranched alkene chain.

39. The peptide according to any one of the preceding items, wherein X₁ and/or X₂ are alkenylated Ser, such as O-alkenylated Ser.

40. The peptide according to any one of items 30 to 39, wherein said alkenylated amino acids comprise 2 to 10 carbons in the alkenyl chain, such as 2, 3, 4, 5, 6, 7, 8, 9, or 10 carbons, preferably 4, 5 or 6 carbons.

41. The peptide according to any one of items 30 to 40, wherein the alkenylated amino acids are α-substituted alkenyl olefin-terminated amino acids and/or α,α-disubstituted alkenyl olefin-terminated amino acids.

42. The peptide according to any one of the preceding items, wherein X₁ and/or X₂ are α,α-disubstituted S- or R-pentenylalanine (S5 or R5) and S- or R-octenylalanine (S8 or R8) alanine.

43. The peptide according to any one of the preceding items, wherein the internal hydrocarbon staple is formed by linking two (S)-2-(4′-pentenyl)-alanines.

44. The peptide according to any one of the preceding items, wherein the covalent linkage is established through ring-closing.

45. The peptide according to any one of the preceding items, wherein the covalent linkage is established through ring-closing metathesis (RCM).

46. The peptide according to any one of the preceding items, wherein X₁ is a non-canonical azido terminated amino acid and X₂ is a non-canonical yne-terminated amino acid or vice versa.

47. The peptide according to any one of the preceding items, wherein X₃ and X₄ are closer to the N-terminal relative to X₁ and X₂.

48. The peptide according to any one of the preceding items, wherein amino acid X₃ is positioned at position n, and amino acid X₄ is positioned at position n+3, or at position, n+4, or at position n+5, wherein n is an integer. In preferred embodiments, amino acid X₃ is positioned at position n, and amino acid X₄ is positioned at position, n+4.

49. The peptide according to any one of the preceding items, wherein amino acid X₃ is positioned at the very N-terminus of the peptide.

50. The peptide according to any one of the preceding items, wherein amino acid X₃ is positioned at the very N-terminus of the peptide and amino acid X₄ is positioned at position n+4.

51. The peptide according to any one of the preceding items, wherein the internal covalent bond is an amide bond formed by reacting an amine and a carboxylic acid.

52. The peptide according to any one of the preceding items, wherein the internal covalent bond is a lactam bridge formed by reacting an amine and a carboxylic acid.

53. The peptide according to any one of the preceding items, wherein X₃ is an N-terminal amino acid harbouring an N-terminal amine group, and X₄ is an amino acid harbouring a carboxylic acid side chain, and wherein X₃ and X₄ are linked by an internal covalent bond formed by reacting said amine with said carboxylic acid.

54. The peptide according to any one of the preceding items, wherein when aligning the sequence of the peptide of the invention to the sequence of GKY25 of SEQ ID NO: 12, then X₃ and X₄ corresponds to Gly1 and Phe5 of GKY25 of SEQ ID NO: 12.

55. The peptide according to any one of the preceding items, wherein X₃ and X₄ corresponds to Gly1 and Glu5 of SEQ ID NO: 14.

56. The peptide according to any one of the preceding items, wherein the peptide has a length between 14 and 22 amino acids, such as between 15 and 21 amino acids, such as between 16 and 20 amino acids, such as between 17 and 20 amino acids.

57. The peptide according to any one of the preceding items, wherein the peptide has a length of 18 to 20 amino acids, such as 18 or 19 amino acids.

58. The peptide according to any one of the preceding items, wherein the peptide comprises a consecutive sequence of amino acids from thrombin of SEQ ID NO: 1 of between 14 and 22 amino acids, such as between 13 and 18 amino acids, such as between 15 and 21 amino acids, such as between 16 and 20 amino acids, such as between 17 and 20 amino acids, preferably between 17 and 18 amino acids.

59. The peptide according to any one of the preceding items, wherein the peptide has a length between 24 and 40 amino acids, such as between 25 and 35 amino acids, such as between 25 and 30 amino acids, such as between 28 and 34 amino acids.

60. The peptide according to any one of the preceding items, wherein the peptide comprises or consists of a consecutive sequence of amino acids from thrombin of SEQ ID NO: 1 of in the range of 16 to 21 amino acids containing up to 6 amino acid substitutions.

61. The peptide according to any one of the preceding items, wherein the peptide comprises or consists of a consecutive sequence of amino acids from thrombin of SEQ ID NO: 1 of in the range of 17 to 18 amino acids containing up to 2 amino acid substitutions, where in peptide may comprise up to 4 additional amino acids.

62. The peptide according to any one of the preceding items, wherein the peptide comprises or consists of a consecutive sequence of amino acids from thrombin of SEQ ID NO: 1 of in the range of 17 to 18 amino acids containing a substitution of one amino acid for amino acid X₁ and a substitution of one amino acid for amino acid X₂, where in peptide may comprise up to 4 additional amino acids.

63. The peptide according to any one of the preceding items, wherein said peptide comprises at least 2 amino acid substitutions, such as 3 amino acid substitutions, such as 4 amino acid substitutions, such as 5 amino acids substitutions compared to the consecutive sequence of thrombin.

64. The peptide according to any one of the preceding items, wherein one or more of said substitutions are conservative substitutions, such as wherein 1, 2, 3 or 4 amino acid substitutions are conservative substitutions.

65. The peptide according to any one of the preceding items, wherein said peptide further comprises one or more moieties conjugated to said peptide, optionally wherein the peptide and the one or more moieties are conjugated to each other by a linker, wherein the one or more moieties are selected from the group consisting of alkyls, aryls, heteroaryls, olefins, fatty acids, polyethylene glycol (PEG), saccharides, and polysaccharides.

66. The peptide according to any one of the preceding items, wherein said peptide further comprises between 1 and 5, such as between 1 and 4, for example between 1 and 3, for example between 1 and 2, such as 2 positively charged amino acids inserted at or close to the end of the peptide.

67. The peptide according to item 66, wherein said positively charged amino acids are inserted at or close to the N-terminal, such as at a position selected from positions 1, 2, 3, 4 and/or 5 relative to the N-terminal of the peptide.

68. The peptide according to any one of items 66 to 67, wherein said positively charged amino acids are inserted at the N-terminal.

69. The peptide according any one of the preceding items, wherein said peptide comprises 2 positively charged amino acids inserted at or close to the N-terminal, such as at a position selected from positions 1, 2, and/or 3 relative to the N-terminal of the peptide.

70. The peptide according to any one of the preceding items, wherein the peptide consists of in the range of 15 to 20 consecutive amino acids of thrombin of SEQ ID NO: 1, wherein 2 amino acids has been substituted with alkenylated amino acids forming an internal hydrocarbon staple, and in the range of 2 to 5 additional N-terminal amino acids

71. The peptide according to any one of the preceding items, wherein the peptide consists of in the range of 16 to 18 consecutive amino acids of thrombin of SEQ ID NO: 1, wherein 2 amino acids has been substituted with alkenylated amino acids forming an internal hydrocarbon staple, and in the range of 2 to 5 additional N-terminal amino acids.

72. The peptide according to any one of the preceding items, wherein the peptide consists of 17 consecutive amino acids of thrombin of SEQ ID NO: 1, wherein 2 amino acids has been substituted with alkenylated amino acids forming an internal hydrocarbon staple, and in the range of 2 to 3 additional N-terminal amino acids.

73. The peptide according to any one of items 66 to 72, wherein the additional N-terminal amino acids are positively charged.

74. The peptide according to any one of items 66 to 73, wherein the positively charged amino acids are selected form the group consisting of arginine, lysine and histidine, preferably wherein the positively charged amino acids are arginine and/or lysine, even more preferably wherein the positively charged amino acids are lysine.

75. The peptide according to any one of the preceding items, wherein the alkenylated amino acids are as defined in any one of items 30 to 43.

76. The peptide according to any one of the preceding items, wherein the peptide comprises the sequence:

-K-K-Z-Z-X₁-K-Z-Z-X₂-Z
wherein
each Z individually is any canonical amino acid; and
X₁ and X₂ are amino acids, the side chains of which are linked by a covalent linkage.

77. The peptide according to any one of the preceding items, wherein the peptide comprises the sequence:

U-U-(Z)ₙ-K-K-Z-Z-X₁-K-Z-Z-X₂-Z,
wherein
each Z individually is any canonical amino acid;
U is His, Lys or Arg;
n is an integer in the range of 0 to 10 and
X₁ and X₂ are amino acids, the side chains of which are linked by a covalent linkage.

78. The peptide according to any one of the preceding items, wherein the peptide comprises the sequence:

Z-R-Z-K-K-Z-Z-X₁-K-Z-Z-X₂-Z
wherein
each Z individually is any canonical amino acid; and
X₁ and X₂ are amino acids linked by a covalent linkage.

79. The peptide according to any one of the preceding items, wherein the peptide comprises or consists of the sequence:

U-U-Z-Z-R-Z-K-K-Z-Z-X₁-K-Z-Z-X₂-Z
wherein
each Z individually is any canonical amino acid;
U is His, Arg or Lys; and
X₁ and X₂ are amino acids linked by a covalent linkage.

80. The peptide according to any one of the preceding items, wherein the peptide comprises or consists of the sequence:

V-F-R-L-K-K-W-I-X₁-K-V-I-X₂-Q-F-G-E
wherein
X₁ and X₂ are amino acids linked by a covalent linkage.

81. The peptide according to any one of the preceding items, wherein the peptide comprises or consists of the sequence:

U-U-V-F-R-L-K-K-W-I-X₁-K-V-I-X₂-Q-F-G-E
wherein
U is His, Arg or Lys; and
X₁ and X₂ are amino acids linked by a covalent linkage.

82. The peptide according to any one of items 76 to 81, wherein U is Lys or Arg.

83. The peptide according to any one of the previous items, wherein the peptide comprises or consists of the sequence as set forth in:
i) SEQ ID NO: 3
ii) SEQ ID NO: 4
iii) SEQ ID NO: 5
iv) SEQ ID NO: 6
v) SEQ ID NO:7
vi) SEQ ID NO: 8;
vii) SEQ ID NO: 9;
viii) SEQ ID NO: 10;
ix) SEQ ID NO: 11
x) SEQ ID NO:17;
xi) SEQ ID NO: 18;
xii) SEQ ID NO: 19;
xiii) SEQ ID NO: 20;
xiv) SEQ ID NO: 21;
xv) SEQ ID NO: 22;
xvi) SEQ ID NO: 23;
xvii) SEQ ID NO: 24;
xviii) SEQ ID NO: 25; or
xix) SEQ ID NO: 26
wherein X₁ and X₂ are amino acids linked by a covalent linkage, and wherein X₃ and X₄ are amino acids linked by a covalent linkage.

84. The stabilized peptide according to any one of the previous items, wherein the peptide comprises or consists of the sequence as set forth in:
iv) SEQ ID NO: 3
v) SEQ ID NO: 4
vi) SEQ ID NO: 5
vii) SEQ ID NO: 8; or
viii) SEQ ID NO: 9
   wherein X₁ and X₂ are amino acids linked by a covalent linkage.

85. The peptide according to any one of the preceding items, wherein said peptide comprises or consists of the sequence as set forth in:
i) SEQ ID NO: 3;
ii) SEQ ID NO: 5;
iii) SEQ ID NO: 6; or
iv) SEQ ID NO: 7;
wherein X₁ and X₂ are amino acids, the side chains of which are linked by a covalent linkage, optionally wherein X₁ and X₂ are (S)-2-(4′-pentenyl)-alanines, the side chains of which have been reacted with each other to form an alkenyl tether, further optionally wherein X₁ and X₂ are Ala, the side chains of which are covalently bound to each other by a C₈ alkenyl tether comprising one double bond.

86. The peptide according to any one of the preceding items, wherein said peptide comprises or consists of the sequence as set forth in:
i) SEQ ID NO: 3
ii) SEQ ID NO: 5; or
   wherein X₁ and X₂ are amino acids, the side chains of which are linked by a covalent linkage, optionally wherein X₁ and X₂ are (S)-2-(4′-pentenyl)-alanines, the side chains of which have been reacted with each other to form an alkenyl tether, further optionally wherein X₁ and X₂ are Ala, the side chains of which are covalently bound to each other by a C₈ alkenyl tether comprising one double bond.

87. The peptide according to any one of the preceding items, wherein said peptide comprises or consists of the sequence as set forth in:
i) SEQ ID NO: 14; or
ii) SEQ ID NO: 26
   wherein X₁ and X₂ are amino acids, the side chains of which are linked by a covalent linkage; and further wherein X₃ and X₄ are amino acids, the side chains of which are linked by a covalent linkage, preferably wherein X₁ and X₂ are (S)-2-(4′-pentenyl)-alanines, the side chains of which have been reacted with each other to form an alkenyl tether, and X₃ and X₄ are Gly and Glu, respectively, which have been reacted with each other to form a lactam bridge.

88. The peptide according to any one of items 49 to 55, wherein X₁ and X₂ are as defined in any one of items 13 to 47.

89. The peptide according to any one of the preceding items, wherein X₃ and X₄ are as defined in any one of items 13 to 55.

90. The peptide according to any one of the preceding items, wherein the peptide contains at least one alpha helical secondary structure unit in aqueous solution.

91. The peptide according to item 90, wherein the alpha helical secondary structure unit is maintained upon binding a target molecule, such as cluster differentiation 14 (CD14).

92. The peptide according to any one of the preceding items, wherein the peptide has a hydrodynamic radii of less than 150 nm, such as less than 100 nm at pH 7.4 and/or less than 100 nm, such as less than 80 nm at pH 5.

93. The peptide according to any one of the preceding items, wherein the peptide has increased stability in vivo and/or in vitro compared to a peptide with the same sequence except that the amino acids X₁ and X₂ are exchanged for other amino acids lacking the covalent linkage, when said peptides are tested under the same conditions.

94. The peptide according to any one of the preceding items, wherein the peptide has increased stability in the presence of a serine protease, such as, compared to a peptide of same sequence except that the amino acids X₁ and X₂ are exchanged for other amino acids lacking the covalent linkage, when said peptides are tested under the same conditions.

95. The peptide according to any one of the preceding items, wherein 50 μM of the peptide has a hemolytic activity in fresh, whole blood of at the most 5%, such as at the most 4%, for example at the most 3%, such as at the most 2%.

96. The peptide according to any one of the preceding items, the peptide has a hemolytic activity in fresh, whole blood of at the most 10%, preferably of at the most 5% at a peptide concentration reducing release of TNF-α in LPS stimulated blood in vitro by 50%.

97. The peptide according to any one of the preceding items, wherein the peptide has anti-coagulant activity.

98. The peptide according to any one of the preceding items, wherein the peptide increases the clotting time as determined by aPPT by at least 100% at a concentration of 60 μM and/or increases the clotting time as determined by aPPT by at least 90% at a concentration of 40 μM peptide.

99. The peptide according to any one of the preceding items, wherein the peptide reduces inflammation and/or infection.

100. The peptide according to any one of the preceding items, wherein the peptide reduces the secretion of pro-inflammatory cytokines in the presence of one or more endotoxins, such as LPS.

101. The peptide according to any one of the preceding items, wherein the peptide decreases secretion of pro-inflammatory cytokines in vivo in blood comprising one or more endotoxins, such as LPS

102. The peptide according to any one of items 100 or 102, wherein said pro-inflammatory cytokines are selected from the group consisting of tumour necrosis factor α (TNF-α), interleukin β (IL-1β), interleukin 6 (IL-6), interleukin 10 (IL-10), interferon (IFN-Y) and/or monocyte chemoattractant protein-1 (MCP-1).

103. The peptide according to any one of the preceding items, wherein the peptide decreases NF-kB activation in the presence of a toll-like receptor (TLR)-agonists, such as lipopolysaccharide (LPS), lipoteichoic acid (LTA), Staphylococcus aureus peptidoglycan (SA-PGN) and/or zymosan.

104. The peptide according to any one of the preceding items, wherein 10 μM of peptide reduces secretion of TNF-α and or IL-1β after incubation in fresh blood in the presence of LPS by at least 50%, such as by at least 60%, for example by at least 70%, compared to a peptide of same sequence except that the amino acids X₁ and X₂ are exchanged for other amino acids lacking the covalent linkage, when said peptides are tested under the same conditions.

105. The peptide according to any one of the preceding items, wherein the peptide is bactericidal, for example wherein the peptide is capable of killing bacteria by damaging the bacterial membrane.

106. The peptide according to item 105, wherein said bacteria is selected from the group consisting of Staphylococcus aureus, Pseudomonas aeruginosa and Escherichia coli.

107. The peptide according to any one of the preceding items, wherein the anti-inflammatory effect of the peptide is maintained 24 h in vivo after systemic administration of the peptide.

108. A peptide according to any one of the preceding items for use as a medicament.

109. A peptide according to any one of the preceding items for use in a method of treatment and/or prevention of inflammation and/or infection in an individual in need thereof.

110. The peptide for use according to item 109, wherein said inflammation is associated with an infection.

111. A peptide according to any one of items 1 to 108 for use in a method of treatment or prevention of a disease selected from the group consisting of acute inflammation, sepsis, acute respiratory distress syndrome (ARDS), chronic obstructive pulmonary disease (COPD), cystic fibrosis, asthma, allergic and other types of rhinitis, vasculitis, thrombosis, disseminated intravascular coagulation (DIC) gastroenteritis, and pulmonary inflammation.

112. The peptide for use according to any one of items 109 to 110, wherein said inflammation is selected from the group consisting of acute respiratory distress syndrome (ARDS), severe acute respiratory syndrome (SARS), gastroenteritis, and pulmonary inflammation.

113. The peptide for use according to any one of items 109 to 110, wherein said inflammation is ARDS.

114. The peptide for use according to any one of items 109 to 113, wherein said treatment is systemic.

115. The peptide for use according to any one of items 109 to 114, wherein said method comprises parental administration of the peptide, such as subcutaneous or intravenous administration.

116. The peptide for use according to any one of items 109 to 115, wherein the individual is a human being.

117. The peptide for use according to any one of items 109 to 116, wherein said infection is infection with a microorganism, such as bacterial infection or a viral infection.

118. The peptide for use according to any one of items 109 to 117, wherein said individual suffers from a bacterial infection.

119. The peptide for use according to item 118, wherein said bacterial infection is an acute or chronic bacterial infection, for example an infection by gram-negative bacteria.

120. The peptide for use according to any one of items 109 to 119, wherein said individual has an increased level of endotoxin, such as an increased level of LPS, LTA and/or SA-PGN.

121. The peptide for use according to any one of items 109 to 120, wherein said individual has an increased level of endotoxin in one or more body fluids, optionally wherein said body fluid is selected from the group consisting of blood, serum, saliva, nasopharyngeal swab samples and bronchoalveolar lavage (BAL) samples, further optionally wherein said endotoxin is LPS.

122. The peptide for use according to any one of items 109 to 121, wherein said increased level of LPS is a level of at least 50 pg/ml, such as a serum level of LPS of at least 50 pg/ml.

123. The peptide for use according to any one of items 109 to 122, wherein the individual suffers from a viral infection.

124. The peptide for use according to item 123, wherein the viral infection is infection with an S protein virus, for example a virus of the Coronaviridae family, such as a virus selected from the group consisting of the virus is selected from the group consisting of:
PorCov-HKU15,
SARS-CoV,
HCoV NL63
HKU1,
MERS-CoV
SARS-CoV 2, and
MERS-CoV.

125. A method of treatment and/or prevention of inflammation and/or infection in an individual in need thereof, said method comprising administering a therapeutically effective amount of the peptide according to any one of items 1 to 107 to said individual.

126. The method according to item 125, wherein the treatment, the inflammation, the infection and/or the individual is a defined in any one of items 110 to 124.

127. Use of a peptide according to any one of items 1 to 107 for the preparation of a medicament for treatment and/or prevention of inflammation and/or infection in an individual in need thereof.

128. The use according to item 127, wherein the treatment, the inflammation, the infection and/or the individual is a defined in any one of items 110 to 124.

129. A pharmaceutical composition comprising the peptide according to any of items 1 to 107.

1. A peptide comprising a consecutive sequence of in the range of 10 to 23 amino acids from thrombin of SEQ ID NO: 1 containing up to 6 amino acid substitutions, wherein said peptide:
i) has a total length between 10 and 40 amino acids;
ii) comprises at least one internal covalent linkage between the side chains of two non-neighbouring, internal amino acids, wherein said amino acids are denoted X₁ and X₂; and
iii) comprises at least amino acids K247, K248 and K252 of thrombin of SEQ ID NO: 1;
with the proviso that if the peptide has a total length between 24 to 40 amino acids it comprises at least two internal covalent linkages between the side chains of two non-neighbouring, internal amino acids, wherein the amino acids of the first internal covalent linkage are denoted X₁ and X₂, and the amino acids of the second internal covalent linkage are denoted X₃ and X₄.

2. The peptide according to claim 1, wherein the peptide comprises at least amino acids R245, K247, K248 and K252 of thrombin of SEQ ID NO: 1.

3. The peptide according to any one of the preceding claims, wherein the peptide has a total length between 10 and 23 amino acids.

4. The peptide according to any one of the preceding claims, wherein the peptide has a total length between 13 and 23 amino acids.

5. The peptide according to any one of the preceding claims, wherein the peptide comprises or consists of the amino acid sequence:
-U-U-(Z)ₙ-I-Q-K-V-I-D-Q-(Z)ₘ-
wherein
each Z individually is any canonical amino acid; and
U is His, Lys or Arg; and
n is an integer in the range of 0 to 10; and
m is an integer in the range of 0 to 5, and
wherein two of the amino acids have been substituted for alkenylated amino acids, the side chains of which are linked by a covalent linkage.

6. The peptide according to claim 1, wherein amino acid X₁ is positioned at position n and amino acid X₂ is positioned at position n+3, or at position, n+4, or at position n+5, or at position n+6, or at position n+7, or at position n+8, or at position n+9, or at position n+10, or at position n+11, wherein n is an integer in the range of 2 to 18.

7. The peptide according to any one of the preceding claims, with the proviso that when aligning the sequence of the peptide to the sequence of GKY25 of SEQ ID NO: 12, then:
a) X₁ does not align to Arg11 in GKY25 of SEQ ID NO: 12
b) X₁ does not align to Lys14 in GKY25 of SEQ ID NO: 12
c) X₂ does not align to Lys14 in GKY25 of SEQ ID NO: 12; and
d) X₂ does not align to Lys 18 in GKY25 of SEQ ID NO: 12; and
e) X₂ does not align to Gln22, when X₁ aligns to Lys18 of SEQ ID NO: 12.

8. The peptide according to any one of the preceding claims, wherein the covalent linkage is a hydrocarbon staple.

9. The peptide according to any one of the preceding claims, wherein X₁ and X₂ are alkenylated amino acids, such as two C-alkenylated amino acids, such as two α-substituted alkenyl amino acids and/or α,α-disubstituted alkenyl amino acids, and the covalent linkage is an olefin tether formed between said alkenyl residues.

10. The peptide according to any one of the preceding claims, wherein X₃ and X₄ are covalently linked by a lactam bridge, optionally wherein said lactam bridge is formed between the N-terminal amine group of X₃ and a side chain carboxylic acid of X₄.

11. The peptide according to any one of the preceding claims, wherein X₁ and/or X₂ are linked by a tether, wherein the tether is an alkene chain of 10 carbon atoms counting from the C-alpha carbon.

12. The peptide according to any one of the preceding claims, wherein the internal hydrocarbon staple is formed by linking two (S)-2-(4′-pentenyl)-alanines.

13. The peptide according to any one of the preceding claims, wherein the peptide has a length between 14 and 22 amino acids, such as between 13 and 18 amino acids, such as between 15 and 21 amino acids, such as between 16 and 20 amino acids, such as between 17 and 20 amino acids.

14. The peptide according to any one of the preceding claims, wherein said peptide further comprises between 1 and 5, such as between 1 and 4, for example between 1 and 3, for example between 1 and 2, such as 2 positively charged amino acids inserted at or close to the end of the peptide.

15. The peptide according any one of the preceding claims, wherein said peptide comprises 2 positively charged amino acids inserted at or close to the N-terminal, such as at a position selected from positions 1, 2, and/or 3 relative to the N-terminal of the peptide.

16. The peptide according to any one of the preceding claims, wherein the peptide consists of in the range of 15 to 20 consecutive amino acids of thrombin of SEQ ID NO: 1, wherein 2 amino acids have been substituted with alkenylated amino acids forming an internal hydrocarbon staple, and in the range of 2 to 5 additional N-terminal amino acids.

17. The peptide according to any one of the previous claims, wherein the peptide comprises or consists of the sequence as set forth in:
i) SEQ ID NO: 3
ii) SEQ ID NO: 4
iii) SEQ ID NO: 5
iv) SEQ ID NO: 6
v) SEQ ID NO: 7
vi) SEQ ID NO: 8;
vii) SEQ ID NO: 9;
viii) SEQ ID NO: 10;
ix) SEQ ID NO: 11;
x) SEQ ID NO: 14;
xi) SEQ ID NO: 17;
xii) SEQ ID NO: 18;
xiii) SEQ ID NO: 19;
xiv) SEQ ID NO: 20;
xv) SEQ ID NO: 21;
xvi) SEQ ID NO: 22;
xvii) SEQ ID NO: 23;
xviii) SEQ ID NO: 24;
xix) SEQ ID NO: 25; or
xx) SEQ ID NO: 26;
wherein X₁ and X₂ are amino acids linked by a covalent linkage, and wherein X₃ and X₄ are amino acids linked by a covalent linkage.

18. The peptide according to any one of the previous claims, wherein the peptide comprises or consists of the sequence as set forth:
i) SEQ ID NO: 3;
ii) SEQ ID NO: 5;
iii) SEQ ID NO: 6; or
iv) SEQ ID NO: 7,
wherein X₁ and X₂ are amino acids, the side chains of which are linked by a covalent linkage, optionally wherein X₁ and X₂ are (S)-2-(4′-pentenyl)-alanines, the side chains of which have been reacted with each other to form an alkenyl tether, further optionally wherein X₁ and X₂ are Ala, the side chains of which are covalently bound to each other by a C₈ alkenyl tether comprising one double bond.

19. The peptide according to any one of the preceding claims, wherein the peptide comprises or consists of the sequence as set forth:
i) SEQ ID NO: 3;
ii) SEQ ID NO: 5;
wherein X₁ and X₂ are (S)-2-(4′-pentenyl)-alanines, the side chains of which have been reacted with each other to form an alkenyl tether, optionally wherein X₁ and X₂ are Ala, the side chains of which are covalently bound to each other by a C₈ alkenyl tether comprising one double bond.

20. The peptide according to any one of the preceding claims, wherein the peptide has a hemolytic activity in fresh, whole blood of at the most 10%, preferably of at the most 5% at a peptide concentration reducing release of TNF-α in LPS stimulated blood in vitro by 50%.

21. A peptide according to any one of the preceding claims for use as a medicament.

22. A peptide according to any one of the preceding claims for use in a method of treatment and/or prevention of inflammation and/or infection in an individual in need thereof.

23. The peptide according to claim 22, wherein the peptide is for use in a method of treatment or prevention of a disease selected from the group consisting of acute inflammation, sepsis, acute respiratory distress syndrome (ARDS), systemic inflammatory response syndrome (SIRS), chronic obstructive pulmonary disease (COPD), cystic fibrosis, asthma, allergic and other types of rhinitis, vasculitis, thrombosis, disseminated intravascular coagulation (DIC) gastroenteritis, and pulmonary inflammation.


SEQUENCE LISTING (33 sequences)

SEQ ID NO: 1 - protein, 259 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 2 - protein, 18 aa, Homo sapiens
HVFRLKKWIQKVIDQFGE

SEQ ID NO: 3 - protein, 18 aa, synthetic construct
  REGION 1..18: note = Stapled peptide
  SITE 10: note = MISC_FEATURE - (S)-2-(4'pentenyl)-alanine
  SITE 14: note = MISC_FEATURE - (S)-2-(4'pentenyl)-alanine
  CROSSLNK 10..14: note = crosslink via an alkenyl tether
HVFRLKKWIXKVIXQFGE

SEQ ID NO: 4 - protein, 18 aa, synthetic construct
  REGION 1..18: note = Stapled peptide
  SITE 10: note = MISC_FEATURE - (S)-2-(4'pentenyl)-alanine
  SITE 14: note = MISC_FEATURE - (S)-2-(4'pentenyl)-alanine
  CROSSLNK 10..14: note = crosslink via an alkenyl tether
KVFRLKKWIXKVIXQFGE

SEQ ID NO: 5 - protein, 19 aa, synthetic construct
  REGION 1..19: note = Stapled peptide
  SITE 11: note = MISC_FEATURE - (S)-2-(4'pentenyl)-alanine
  SITE 15: note = MISC_FEATURE - (S)-2-(4'pentenyl)-alanine
  CROSSLNK 11..15: note = crosslink via an alkenyl tether
KKVFRLKKWIXKVIXQFGE

SEQ ID NO: 6 - protein, 20 aa, synthetic construct
  REGION 1..20: note = Stapled peptide
  SITE 12: note = MISC_FEATURE - (S)-2-(4'pentenyl)-alanine
  SITE 16: note = MISC_FEATURE - (S)-2-(4'pentenyl)-alanine
  CROSSLNK 12..16: note = crosslink via an alkenyl tether
KKKVFRLKKWIXKVIXQFGE

SEQ ID NO: 7 - protein, 21 aa, synthetic construct
  REGION 1..21: note = Stapled peptide
  SITE 13: note = MISC_FEATURE - (S)-2-(4'pentenyl)-alanine
  SITE 17: note = MISC_FEATURE - (S)-2-(4'pentenyl)-alanine
  CROSSLNK 13..17: note = crosslink via an alkenyl tether
KKKKVFRLKKWIXKVIXQFGE

SEQ ID NO: 8 - protein, 18 aa, synthetic construct
  REGION 1..18: note = Stapled peptide
  SITE 10: note = MISC_FEATURE - (S)-2-(4'pentenyl)-alanine
  SITE 14: note = MISC_FEATURE - (S)-2-(4'pentenyl)-alanine
  CROSSLNK 10..14: note = crosslink via an alkenyl tether
RVFRLKKWIXKVIXQFGE

SEQ ID NO: 9 - protein, 19 aa, synthetic construct
  REGION 1..19: note = Stapled peptide
  SITE 11: note = MISC_FEATURE - (S)-2-(4'pentenyl)-alanine
  SITE 15: note = MISC_FEATURE - (S)-2-(4'pentenyl)-alanine
  CROSSLNK 11..15: note = crosslink via an alkenyl tether
RRVFRLKKWIXKVIXQFGE

SEQ ID NO: 10 - protein, 20 aa, synthetic construct
  REGION 1..20: note = Stapled peptide
  SITE 12: note = MISC_FEATURE - (S)-2-(4'pentenyl)-alanine
  SITE 16: note = MISC_FEATURE - (S)-2-(4'pentenyl)-alanine
  CROSSLNK 12..16: note = crosslink via an alkenyl tether
RRRVFRLKKWIXKVIXQFGE

SEQ ID NO: 11 - protein, 21 aa, synthetic construct
  REGION 1..21: note = Stapled peptide
  SITE 13: note = MISC_FEATURE - (S)-2-(4'pentenyl)-alanine
  SITE 17: note = MISC_FEATURE - (S)-2-(4'pentenyl)-alanine
  CROSSLNK 13..17: note = crosslink via an alkenyl tether
RRRRVFRLKKWIXKVIXQFGE

SEQ ID NO: 12 - protein, 25 aa, Homo sapiens
GKYGFYTHVFRLKKWIQKVIDQFGE

SEQ ID NO: 13 - protein, 25 aa, synthetic construct
  REGION 1..25: note = Stapled peptide
  SITE 17: note = MISC_FEATURE - (S)-2-(4'pentenyl)-alanine
  SITE 21: note = MISC_FEATURE - (S)-2-(4'pentenyl)-alanine
  CROSSLNK 17..21: note = crosslink via an alkenyl tether
GKYGFYTHVFRLKKWIXKVIXQFGE

SEQ ID NO: 14 - protein, 25 aa, synthetic construct
  REGION 1..25: note = Stapled peptide
  REGION 1..5: note = MISC_FEATURE - Ciclo
  SITE 17: note = MISC_FEATURE - (S)-2-(4'pentenyl)-alanine
  SITE 21: note = MISC_FEATURE - (S)-2-(4'pentenyl)-alanine
  CROSSLNK 17..21: note = crosslink via an alkenyl tether
  CROSSLNK 1..5: note = a lactam bridge which is formed between the N-terminal amine group of G and a side chain carboxylic acid of E
GKYGEYTHVFRLKKWIXKVIXQFGE

SEQ ID NO: 15 - protein, 21 aa, Homo sapiens
FYTHVFRLKKWIQKVIDQFGE

SEQ ID NO: 16 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 17 - protein, 17 aa, synthetic construct
  CROSSLNK 9..13: note = crosslink via an alkenyl tether
  SITE 9: note = (S)-2-(4'pentenyl)-alanine
  SITE 13: note = (S)-2-(4'pentenyl)-alanine
VFRLKKWIXKVIXQFGE

SEQ ID NO: 18 - protein, 16 aa, synthetic construct
  CROSSLNK 8..12: note = crosslink via an alkenyl tether
  SITE 8: note = (S)-2-(4'pentenyl)-alanine
  SITE 12: note = (S)-2-(4'pentenyl)-alanine
FRLKKWIXKVIXQFGE

SEQ ID NO: 19 - protein, 15 aa, synthetic construct
  CROSSLNK 7..11: note = crosslink via an alkenyl tether
  SITE 7: note = (S)-2-(4'pentenyl)-alanine
  SITE 11: note = (S)-2-(4'pentenyl)-alanine
RLKKWIXKVIXQFGE

SEQ ID NO: 20 - protein, 14 aa, synthetic construct
  CROSSLNK 6..10: note = crosslink via an alkenyl tether
  SITE 6: note = (S)-2-(4'pentenyl)-alanine
  SITE 10: note = (S)-2-(4'pentenyl)-alanine
LKKWIXKVIXQFGE

SEQ ID NO: 21 - protein, 13 aa, synthetic construct
  CROSSLNK 5..9: note = crosslink via an alkenyl tether
  SITE 5: note = (S)-2-(4'pentenyl)-alanine
  SITE 9: note = (S)-2-(4'pentenyl)-alanine
KKWIXKVIXQFGE

SEQ ID NO: 22 - protein, 14 aa, synthetic construct
  CROSSLNK 6..10: note = crosslink via an alkenyl tether
  SITE 6: note = (S)-2-(4'pentenyl)-alanine
  SITE 10: note = (S)-2-(4'pentenyl)-alanine
KKKWIXKVIXQFGE

SEQ ID NO: 23 - protein, 15 aa, synthetic construct
  CROSSLNK 7..11: note = crosslink via an alkenyl tether
  SITE 7: note = (S)-2-(4'pentenyl)-alanine
  SITE 11: note = (S)-2-(4'pentenyl)-alanine
KKKKWIXKVIXQFGE

SEQ ID NO: 24 - protein, 14 aa, synthetic construct
  CROSSLNK 6..10: note = crosslink via an alkenyl tether
  SITE 6: note = (S)-2-(4'pentenyl)-alanine
  SITE 10: note = (S)-2-(4'pentenyl)-alanine
RKKWIXKVIXQFGE

SEQ ID NO: 25 - protein, 15 aa, synthetic construct
  CROSSLNK 7..11: note = crosslink via an alkenyl tether
  SITE 7: note = (S)-2-(4'pentenyl)-alanine
  SITE 11: note = (S)-2-(4'pentenyl)-alanine
RRKKWIXKVIXQFGE

SEQ ID NO: 26 - protein, 25 aa, synthetic construct
  CROSSLNK 1..5: note = lactam bridge which is formed between the N-terminal amine group of G and a side chain carboxylic acid of E
  CROSSLNK 17..21: note = crosslink via an alkenyl tether
  REGION 1..5: note = cyclo
  SITE 17: note = (S)-2-(4'pentenyl)-alanine
  SITE 21: note = (S)-2-(4'pentenyl)-alanine
GKYGEYTHVFRLKKWIXKVIXQFGE

SEQ ID NO: 27 - protein, 13 aa, synthetic construct
  REGION 1..13: note = Stapled peptide
  VARIANT 3: note = MISC_FEATURE - X = any canonical amino acid
  VARIANT 6: note = MISC_FEATURE - X = any canonical amino acid
  VARIANT 13: note = MISC_FEATURE - X = any canonical amino acid
  VARIANT 1: note = MISC_FEATURE - X = any canonical amino acid
  VARIANT 11: note = MISC_FEATURE - X = any canonical amino acid
  VARIANT 7: note = MISC_FEATURE - X = any canonical amino acid
  VARIANT 10: note = MISC_FEATURE - X = any canonical amino acid
  VARIANT 8: note = MISC_FEATURE - X = any amino acid, the side chain of which is linked by a covalent linkage to the side chain of the amino acid at position 12
  VARIANT 12: note = note = MISC_FEATURE - X = any amino acid, the side chain of which is linked by a covalent linkage to the side chain of the amino acid at position 8
XRXKKXXXKXXXX

SEQ ID NO: 28 - protein, 16 aa, synthetic construct
  REGION 1..16: note = Stapled peptide
  VARIANT 9: note = MISC_FEATURE - X = any canonical amino acid
  VARIANT 10: note = MISC_FEATURE - X = any canonical amino acid
  VARIANT 4: note = MISC_FEATURE - X = any canonical amino acid
  VARIANT 14: note = MISC_FEATURE - X = any canonical amino acid
  VARIANT 16: note = MISC_FEATURE - X = any canonical amino acid
  VARIANT 3: note = MISC_FEATURE - X = any canonical amino acid
  VARIANT 13: note = MISC_FEATURE - X = any canonical amino acid
  VARIANT 6: note = MISC_FEATURE - X = any canonical amino acid
  VARIANT 1: note = MISC_FEATURE - X = His, Lys or Arg
  VARIANT 2: note = MISC_FEATURE - X = His, Lys or Arg
  VARIANT 11: note = MISC_FEATURE - X= any amino acid, the side chain of which is linked by a covalent linkage to the side chain of the amino acid at position 15
  VARIANT 15: note = MISC_FEATURE - X= any amino acid, the side chain of which is linked by a covalent linkage to the side chain of the amino acid at position 11
XXXXRXKKXXXKXXXX

SEQ ID NO: 29 - protein, 18 aa, synthetic construct
  REGION 1..18: note = Stapled peptide
  VARIANT 1: note = MISC_FEATURE - X = His, Lys or Arg
  VARIANT 14: note = MISC_FEATURE - X= any amino acid, the side chain of which is linked by a covalent linkage to the side chain of the amino acid at position 10
  VARIANT 15: note = MISC_FEATURE - X = any canonical amino acid
  VARIANT 18: note = MISC_FEATURE - X = any canonical amino acid
  VARIANT 10: note = MISC_FEATURE - X= any amino acid, the side chain of which is linked by a covalent linkage to the side chain of the amino acid at position 14
XVFRLKKWIXKVIXXFGX

SEQ ID NO: 30 - protein, 17 aa, synthetic construct
  REGION 1..17: note = Stapled peptide
  VARIANT 13: note = MISC_FEATURE - X= any amino acid, the side chain of which is linked by a covalent linkage to the side chain of the amino acid at position 9
  VARIANT 9: note = MISC_FEATURE - X= any amino acid, the side chain of which is linked by a covalent linkage to the side chain of the amino acid at position 13
VFRLKKWIXKVIXQFGE

SEQ ID NO: 31 - protein, 19 aa, synthetic construct
  REGION 1..19: note = Stapled peptide
  VARIANT 2: note = MISC_FEATURE - X = His, Lys or Arg
  VARIANT 1: note = MISC_FEATURE - X = His, Lys or Arg
  VARIANT 16: note = MISC_FEATURE - X = any canonical amino acid
  VARIANT 19: note = MISC_FEATURE - X = any canonical amino acid
  VARIANT 15: note = MISC_FEATURE - X= any amino acid, the side chain of which is linked by a covalent linkage to the side chain of the amino acid at position 11
  VARIANT 11: note = MISC_FEATURE - X= any amino acid, the side chain of which is linked by a covalent linkage to the side chain of the amino acid at position 15
XXVFRLKKWIXKVIXXFGX

SEQ ID NO: 32 - protein, 19 aa, synthetic construct
  REGION 1..19: note = Stapled peptide
  VARIANT 11: note = MISC_FEATURE - X= any amino acid, the side chain of which is linked by a covalent linkage to the side chain of the amino acid at position 15
  VARIANT 15: note = MISC_FEATURE - X= any amino acid, the side chain of which is linked by a covalent linkage to the side chain of the amino acid at position 11
  VARIANT 2: note = MISC_FEATURE - X = His, Lys or Arg
  VARIANT 1: note = MISC_FEATURE - X = His, Lys or Arg
XXVFRLKKWIXKVIXQFGE

SEQ ID NO: 33 - protein, 11 aa, synthetic construct
  REPEAT 3: note = The amino acid is repeated n times, wherein n is an integer in the range of 0 to 10
  REPEAT 11: note = the amino acid is repeated m times, wherein m is an integer in the range of 0 to 5
  REGION 1..11: note = the peptide has a total length between 10 to 23 amino acids, wherein two of the amino acids have been substituted for alkenylated amino acids, the side chains of which are linked by a covalent linkage
  VARIANT 2: note = U is His, Lys or Arg
  VARIANT 1: note = U is His, Lys or Arg
UUXIQKVIDQX